(19) United States
(12) Patent Application Publication
(10) Pub. No.: US 2007/0059288 A1
(43) Pub. Date: Mar. 15, 2007

(54) Treatment for heart disease

(76) Inventors: Jonathan H. Dinsmore, Brookline, MA (US); Douglas B. Jacoby, Wellesley, MA (US)

Correspondence Address: CHOATE, HALL & STEWART LLP, TWO INTERNATIONAL PLACE, BOSTON, MA, 02110, US

(21) Appl. No.: 11/394,537

(22) Filed: Mar. 31, 2006

Related U.S. Application Data
(60) Provisional application No. 60/666,932, filed on Mar. 31, 2005.

(51) Int. Cl. A61K 48/00; A61K 35/14; A61K 38/17

(52) U.S. Cl. 424/93.2; 424/93.7; 514/2

(57) ABSTRACT

The present invention provides a system for treating heart disease using a combination of pro-angiogenesis therapy and cellular cardiomyoplasty. The system is particularly useful in treating patients with damaged myocardium due coronary artery disease, myocardial infarction, congestive heart failure, and ischemia. A pro-angiogenic factor (e.g., VEGF) or a means of delivering a pro-angiogenic factor (e.g., a genetically engineered adenovirus, adeno-asssociated virus, or cells) is administered to the heart in order to promote new blood vessel growth in an ischemic or damaged area of the patient's heart. Cells such as skeletal myoblasts or stem cells (e.g., mesenchymal stem cells) with the potential to divide, differentiate, and integrate themselves into the injured myocardium are then administered into the affected area of the heart. By inducing new blood vessels growth in the injured myocardium, the cells are better able to grow and become an integral part of the heart. The invention also provides kits for use in treating a patient using the inventive method. Such kits may contain cells, catheters, syringes, needles, cell culture materials, polynucleotides, media, buffers, etc.

### RELATED APPLICATIONS

The present application claims priority under 35 U.S.C. § 119(e) to U.S. provisional patent application, U.S. Ser. No. 60/666,932, filed Mar. 31, 2005, which is incorporated herein by reference.

### BACKGROUND OF THE INVENTION

Despite dramatic advances in the treatment of heart disease over the past three decades, coronary artery disease (CAD) remains the leading cause of death in the Western world (“Mortality from coronary heart disease and acute myocardial infarction” Morbidity & Mortality Weekly Report 50:90-93, 2001; incorporated herein by reference). More specifically, while preventative measures and “mechanical” revascularization strategies (angioplasty and bypass surgery) have resulted in five year survival rates in excess of 80% for individuals who are candidates for such therapies, treatment options remain limited when coronary disease has progressed to diffuse, occlusive disease, and/or infarction (American Heart Association, Heart and Stroke Statistical Update, 2003; incorporated herein by reference). The two-year survival rate for individuals with such advanced coronary artery disease is as low as 20% (Anyanwu et al. “Prognosis after heart transplantation: transplants alone cannot be the solution for end stage heart failure” BMJ 326:509-510, 2003; incorporated herein by reference).

Each year, almost 1.1 million Americans suffer an acute myocardial infarction (American Heart Association, Heart and Stroke Statistical Update, 2003; incorporated herein by reference). Early intervention can limit infarct size and improve early survival (Mitchell et al. “Left ventricular remodeling in the year after first anterior myocardial infarction: a quantitative analysis of contractile segment lengths and ventricular shape” J Am. Coll. Cardiol 19:1136-44, 1992; Migrino et al. “End-systolic volume index at 90 and 180 minutes into reperfusion therapy for acute myocardial infarction is a strong predictor of early and late mortality” Circulation 96:116-121, 1997; Boyle et al. “Limitation of infarct expansion and ventricular remodeling by late reperfusion. Study of time course and mechanism in a rat model” Circulation 88:2872-83, 1993; each of which is incorporated herein by reference). However, 20% of those patients surviving an acute myocardial infarction will develop significant left ventricular dilatation with a left ventricular end-systolic volume index (LVESVI) of less than 60 mL/m². The GUSTO I trial (Migrino et al. “End-systolic volume index at 90 and 180 minutes into reperfusion therapy for acute myocardial infarction is a strong predictor of early and late mortality” Circulation 96:116-121, 1997; incorporated herein by reference) documented that left ventricular dilatation following myocardial infarction is an independent and significant predictor of mortality. Therefore, whereas early survival after myocardial infarction may be predicated by the timeliness and adequacy of appropriate reperfusion therapy, long-term prognosis is strongly dependent on subsequent changes in left ventricular geometry and function These are the determinants of congestive heart failure (Mitchell et al. “Left ventricular remodeling in the year after first anterior myocardial infarction: a quantitative analysis of contractile segment lengths and ventricular shape” J Am. Coll. Cardiol. 19:1136-44, 1992; Gheorghiade et al. “Chronic heart failure in the United States, a manifestation of coronary artery disease” Circulation. 97:282-89, 1998; White et al. “Left ventricular end-systolic volume as the major determinant of survival after recovery from myocardial infarction” Circulation 76(1):44-51, 1987; each of which is incorporated herein by reference).

Congestive heart failure (CHF), which can result from an acute myocardial infarction, currently affects over 5 million people in the United States (National Heart Lung and Blood Institute National Institutes of Health Data Fact Sheet: congestive heart failure in the United States: A new epidemic. NHLBI web site. www/nhlbi.nih.gov/health/public/heart/other/chf.htm; O'Connell et al. “Economic impact of heart failure in the United States: time for a different approach” J. Heart Lung Transplant. 13:S107-S112, 1994; each of which is incorporated herein by reference). Medical therapies, despite some progress, still confer only a <50% one-year survival in patients with the most severe clinical manifestations of end-stage CHF (Rose et al. “Long-term use of a left ventricular assist device for end-stage heart failure” NEJM 345(20):1435-43, 2001; incorporated herein by reference). Despite its clinical effectiveness, heart transplantation is a therapy with little epidemiological significance in the fight against heart failure (Taylor et al. “The registry of the international society of heart and lung transplantation: 20^{th} official adult heart transplant report-2003” J. Heart Lung Transplant. 22(6):616-624, 2003; incorporated herein by reference). As a result, cell-based therapies for repair and regeneration of infarcted myocardium have been proposed to treat patients suffering from chronic heart failure (Chiu et al. “Cellular cardiomyoplasty: myocardial regeneration with satellite cell implantation” Ann. Thor. Surg. 60:12-8, 1995; Pagani et al. “Autologous skeletal myoblasts transplanted to ischemia-damaged myocardium in humans” J. Am. Coll. Cardiol. 41:879-888, 2003; Ghostine et al. “Long-term efficacy of myoblast transplantation on regional structure and function after myocardial infarction” Circulation 106[suppl I]:1131-6, 2002; Dorfman et al. “Myocardial tissue engineering with autologous myoblast implantation” J. Thor. Cardiovasc. Surg. 116:744-51, 1988; Taylor et al. “Regenerating functional myocardium: improved performance after skeletal myoblast transplantation” Nat. Med. 4(8):929-33, 1998; Retuerto et al. “Angiogenic pre-treatment improves the efficacy of cellular cardiomyoplasty performed with fetal cardiomyocyte implantation” J. Thorac. Cardiovasc. Surg. 127:1-11, 2004; Jain et al. “Cell therapy attenuates deleterious ventricular remodeling and improves cardiac performance after myocardial infarction” Circulation 103:1920-27, 2001; Reinecke et al. “Evidence for fusion between cardiac and skeletal muscle cells” Circ. Res. 94(6):e56-60, 2004; McConnell et al. “Correlation of autologous skeletal myoblast survival with changes in left ventricular remodeling in dilated ischemic heart failure” J. Thorac. Cardiovasc. Surg. 2004 (in press); Kessler et al. “Myoblast cell grafting into heart muscle: cellular biology and potential applications” Annu. Rev. Physiol. 61:219-42, 1999; Yoo et al. “Heart cell transplantation improves heart function in dilated cardiomyopathic hamsters” Circulation. 102(19 Suppl 3):III204-9, 2000; Koh et al. “Stable fetal cardiomyocyte grafts in the hearts of dystrophic mice and dogs” J. Clin. Invest. 96(4):2034-42, 1995; Klug et al. “Genetically selected cardiomyocytes from differentiating embronic stem cells form stable intracardiac grafts” J. Clin. Invest. 98(1):216-24, 1996; Jackson et al. “Regeneration of ischemic cardiac muscle and vascular endothelium by adult stem cells” J. Clin. Invest. 107(11): 1395-402, 2001; Kocher et al. “Neovascularization of ischemic myocardium by human bone marrow derived angioblasts prevents cardiomyocyte apoptosis, reduces remodeling and improves cardiac function” Nature Medicine 7:430-436, 2001; Kamihata et al. “Implantation of bone marrow mononuclear cells into ischemic myocardium enhances collateral perfusion and regional function via side supply of angioblasts, angiogenic ligands, and cytokines” Circulation 104:1046-1052, 2001; Orlic et al. “Transplanted adult bone marrow cells repair myocardial infarcts in mice” Ann. N.Y. Acad. Sci. 938:221-9, discussion 229-30, 2001; Orlic et al. “Mobilized bone marrow cells repair the infarcted heart, improving function and survival” Proc. Natl. Acad. Sci. U.S.A. (98):10344-9, 2001; Balsam et al. “Haematopoietic stem cells adopt mature haematopoietic fates in ischaemic myocardium” Nature 428(6983):668-73, 2004; Reinecke et al. “Taking the toll after cardiomyocyte grafting: a reminder of the importance of quantitative biology” J. Mol. Cell. Card. 34:251-253, 2002; Perin et al. “Transendocardial, autologous bone marrow cell transplantation for severe, chronic ischemic heart failure” Circulation 107:2294-2302, 2003; Tse et al. “Angiogenesis in ischaemic myocardium by intramyocardial autologous bone marrow mononuclear cell implantation” Lancet 361 :47-49, 2003; Kamihata et al. “Implantation of bone marrow mononuclear cells into ischemic myocardium enhances collateral perfusion and regional function via side supply of angioblasts, angiogenic ligands, and cytokines” Circulation 104:1046-1052, 2001; each of which is incorporated herein by reference). Immature cells are grafted into the heart in key areas of myocardial dysfunction with the goal of angiogenesis, vasculogenesis, and/or myogenesis to promote functional and geometric restoration. Unfortunately, current results in human clinical trials demonstrate that cellular graft survival number is very poor with typically <1% of autologous myoblasts surviving implantation (Pagani et al. “Autologous skeletal myoblasts transplanted to ischemia-damaged myocardium in humans.” J. Am. Coll. Cardiol. 41:879-888, 2003; incorporated herein by reference). The reason for such poor cell survival and engraftment is unknown.

Cell transfer is generally thought to provide for the regeneration of cardiac function in the setting of myocardial infarction by: (1) “repopulating” scarred myocardium with contractile myocytes; (2) providing a “scaffolding” to diminish further remodeling of the thinned, injured ventricle; or (3) serving as a vehicle for the angiogenic stimulation of ischemic myocardium (Scorsin et al. “Comparison of the effects of fetal cardiomyocyte and skeletal myoblast transplantation on postinfarction left ventricular function” J. Thorac. Cardiovasc. Surg. 119:1169-75, 2000; Jain et al. “Cell therapy attenuates deleterious ventricular remodeling and improves cardiac performance after myocardial infarction” Circulation 103:1920-1927, 2001; Suzuki et al. “Development of a novel method for cell transplantation through the coronary artery” Circulation 102[suppl III]:III-359-III-364, 2000; Orlic et al. “Bone marrow cells regenerate infarcted myocardium” Nature 410:701-704, 2001; Wang et al. “Marrow stromal cells for cellular cardiomyoplasty: feasibility and potential” J. Thorac. Cardiovasc. Surg. 120:999-1006, 2000; Tomita et al. “Autologous transplantation of bone marrow cells improves damaged heart heart function” Circulation 100[suppl II]:II-247-II-256, 1999; Reinecke et al. “Survival, Integration, and differentiation of cardiomyocyte grafts:a study in normal and injured rat hearts” Circulation 100:193-202, 1999; Sakai et al. “Cardiothoracic transplantation. Fetal cell transplantation: a comparison of three cell types” J. Thorac. Cardiovasc. Surg. 118:715-25, 1999; Chedrawy et al. “Incorporation and integration of implanted myogenic and stem cells into native myuocardial fibers: anatomic basis for functional improvements” J. Thorac. Cardiovasc. Surg. 124:584-90, 2002; Klug et al. “Genetically selected cardiomyocytes from differentiating embryonic stem cells form stable intracardiac grafts” J. Clin. Invest. 98:216-224, 1996; Atkins et al. “Intracardiac transplantation of skeletal myoblasts yields two populations of striated cells in situ” Ann. Thorac. Surg. 67:124-9, 1999; Leor et al. “Transplantation of fetal myocardial tissue into the infarcted myocardium of rat. A potential method for repair of infarcted myocardium?” Circulation 94 [suppl II]:II-332-II-336, 1996; Zhang et al. “Cardiomyocyte grafting for cardiac repair: graft cell death and anti-death strategies” J. Mol. Cell. Cardiol. 33:907-921, 2001; Li et al. “Natural history of fetal rat cardiomyocytes transplanted into adult rat myocardial scar tissue” Circulation 96[suppl II]:II-179-II-187, 1997; Taylor et al. “Regenerating functional myocardium: improved performance after skeletal myoblast transplantation” Nature Medicine 4:929-933, 1998; Menasché, “Cell therapy of heart failure” C R Biologies 325:731-738, 2002, Oh et al. “Cardiac progenitors from adult myocardium: homing, differentiation and fusion after infarction” Proc. Natl. Acad. Sci. USA 100:12313-18, 2003; Terada et al. “Bone marrow cells adopt the phenotype of other cells by spontaneous cell fusion” Nature 416:542-5, 2002; Ying et al. “Changing potency by spontaneous fusion” Nature 416:545-8, Apr. 4, 2002; each of which is incorporated herein by reference). It is unknown which if any of these mechanisms is relevant to the putative efficacy of cellular cardiomyoplasty (CCM). In this regard, the typically extremely inefficient (<10%) observed engraftment of cells into areas of myocardial scar has been cited as a potential explanation for the relatively limited improvements in ventricular function noted after cell implantation in animal studies (Pagani et al. “Autologous skeletal myoblasts transplanted to ischemia-damaged myocardium in humans: Histological analysis of cell survival and differentiation” J. Am. Coll. Cardiol. 41:879-88, 2003; Matsushita et al. “Formation of cell junctions between grafted and host cardiomyocytes at the border zone of rat myocardial infarction” Circulation 100[suppl II]:II-262-II-268, 1999; Kehat et al. “Human embryonic stem cells can differentiate into myocytes with structural and functional properties of cardiomyocytes” J. Clin. Invest. 108:407-414, 2001; Boheler et al. “Differentiation of pluripotent embryonic stem cells into cardiomyocytes” Circ. Res. 91:189-201, 2002; Toma et al. “Human mesenchymal stem cells differentiate to a cardiomyocyte phenotype in the adult murine heart” Circulation 105:93-98, 2002; Tambara et al. “Transplanted skeletal myoblasts can fully replace the infracted myocardium when they survive in the host in large numbers” Circulation 108[suppl II]:II-259-II-263, 2003; Minami et al. “Skeletal muscle meets cardiac muscle” J. Am. Coll. Cardiol. 41:1084-6, 2003; Ghostine et al. “Long-term efficacy of myoblast transplantation on regional structure and function after myocardial infarction” Circulation 100[suppl I]:I-131-I-136, 2002; each of which is incorporated herein by reference), and has raised doubts as to the importance of the persistent physical presence of cell implants in myocardial scar, as opposed to their potential role as a transient mediator of angiogenesis (Scorsin et al. “Comparison of the effects of fetal cardiomyocyte and skeletal myoblast transplantation on postinfarction left ventricular function” J. Thorac. Cardiovasc. Surg. 119:1169-75, 2000; Reinecke et al. “Survival, Integration, and differentiation of cardiomyocyte grafts: a study in normal and injured rat hearts” Circulation 100: 193-202, 1999; Zhang et al. “Cardiomyocyte grafting for cardiac repair: graft cell death and anti-death strategies” J. Mol. Cell. Cardiol. 33:907-921, 2001; Menasché, “Cell therapy of heart failure” C. R. Biologies 325:731-738, 2002; each of which is incorporated herein by reference). Also, while the efficacy of CCM has been idealized to involve the differentiation of stem cells into functional cardiomyocytes, evidence of such differentiation may have been confounded by the potential occurrence of cell fusion between implanted stem cells and host myocytes (Oh et al. “Cardiac progenitors from adult myocardium: homing, differentiation and fusion after infarction” Proc. Natl. Acad. Sci. USA 100:12313-18, 2003; Terada et al. “Bone marrow cells adopt the phenotype of other cells by spontaneous cell fusion” Nature 416:542-5, 2002; Ying et al. “Changing potency by spontaneous fusion” Nature 416:545-48, Apr. 4, 2002; each of which is incorporated herein by reference). Finally, while skeletal myoblasts have been demonstrated to provide functional advantages over fibroblast implants in cardiomyoplasty studies, no functional advantages have yet been demonstrated between stem cell and skeletal myoblast implantation, and contractility has not yet been demonstrated in any cell implants (Scorsin et al. “Comparison of the effects of fetal cardiomyocyte and skeletal myoblast transplantation on postinfarction left ventricular function” J. Thorac. Cardiovasc. Surg. 119:1169-75, 2000; Jain et al. “Cell therapy attenuates deleterious ventricular remodeling and improves cardiac performance after myocardial infarction” Circulation 103:1920-1927, 2001; Sakai et al. “Cardiothoracic transplantation. Fetal cell transplantation: a comparison of three cell types” J. Thorac. Cardiovasc. Surg. 118:715-25, 1999; Taylor et al. “Regenerating functional myocardium: improved performance after skeletal myoblast transplantation” Nature Medicine 4:929-933, 1998; each of which is incorporated herein by reference).

There remains a need for a better understanding of cell survival and engraftment in cellular cardiomyoplasty, and for improvement in the success rate of cellular implantation in the heart.

### SUMMARY OF THE INVENTION

The present invention encompasses the recognition that the poor cell survival and engraftment observed in cellular cardiomyoplasty may be due to the hypoxic environment of the tissue into which the cells are being implanted. According to the present invention, cells are implanted into the heart of the patient after pretreatment or concurrent treatment with pro-angiogenic factors. In certain embodiments, the cells to be implanted are engineered to express a pro-angiogenic factor such as VEGF. In some embodiments, anti-apoptotic therapy may be employed to prevent the implanted cells from undergoing apoptosis, e.g., the cells may be engineered to not undergo apoptosis. The inventive treatment improves cardiac function, for example, reversing, preventing, or reducing the remodeling of the heart to prevent LV dilatation and/or reduce LV size(e.g., maintain left ventricular end-systolic index (LVESI) above 60 mL/m²).

In one aspect, the invention includes a method of treating a patient suffering from heart disease (e.g., ischemic heart disease) by implanting cells into the patient's heart and treating the heart with at least one pro-angiogenic factor or a vector encoding at least one pro-angiogenic factor. Typically, treatment with a pro-angiogenic factor (e.g., VEGF) precedes cell implantation. Often an amount of time (e.g., 3 weeks) sufficient to allow the ischemic tissue to revascularize enough to support the newly implanted cells is allowed to elapse before the cells are implanted. Cells that may be used in the inventive method include skeletal myoblasts, mesenchymal stem cells, cardiomyocytes, fetal cardiomyocytes, embryonic stem cells, fibroblasts, pluripotent stem cells, hematopoietic stem cells, cord blood cells, primordial germ cells, neural stem cells, and adult bone marrow-derived stem cells. In certain embodiments, skeletal myoblasts are used. In certain other embodiments, mesenchymal stem cells are implanted into the heart of the patient. In other embodiments, stem cells are implanted. The cells used may be engineered to express a pro-angiogenic factor and/or an anti-apoptotic factor. The cells may be delivered by direct epicardial injection or by catheter based endocardial delivery. The cells may be delivered during a surgical procedure. In certain embodiments, a side port needle is used to implant the cells. Typically, the administration of the cells will follow the pre-treatment with a pro-angiogenic factor by at least 1, 2, 3, 4, or 5 weeks.

In another aspect, the invention provides a method of transplanting cells engineered to express one or more pro-angiogenic factors such as VEGF. Such engineered cells are administered to the patient's heart. The cells may be administerd by direct epicardial injection or by catheter-based endocardial delivery. The cells may be implanted during a surgical procedure. The cells delivered are skeletal myoblasts, mesenchymal stem cells, endothelial stem cells, bone marrow stem cells, hematopoietic stem cells, cord blood cells, primordial germ cells, neural stem cells, pluripotent stem cells, cardiomyocytes, fetal cardiomyocytes, embryonic stem cells, fibroblasts, or adult bone marrow-derived cells. In certain embodiments, the cells are skeletal myoblasts. In certain other embodiments, the cells are mesenchymal stem cells. In certain embodiments, the cells are other types of stem cells (e.g., hematopoietic stem cells). The cells are engineered using techniques known in the art so that they express a pro-angiogenic factor. The pro-angiogenic factor may be constitutively expressed, or expression of the factor may be triggered by a stimulus such as hypoxia, low pH, high CO₂, cell stress, etc. The construct responsible for expression of the factor may be integrated into the genome of the cell or may exist on a separate polynucleotide such as a plasmid, cosmid, artificial chromosome, or viral genome. Optionally, the cells may also be engineered to not undergo apoptosis. Without wishing to be bound by any particular theory, it is proposed that such engineered cells will yield a better rate of survival of the implanted cells. The administration of engineered cells may also be combined with pretreatment with a pro-angiogenic factor or a vector encoding an pro-angiogenic factor as described above.

In another aspect, a kit is provided for practicing the claimed invention. The kit may include combinations of components useful in the practice of the invention such as needles (including side port needles), syringes, catheters, cells, polynucleotides, vectors, engineered adenovirus, enzymes used in molecular biology such as endonucleases, ligases, kinases, etc., buffers, polymeric matrices, pro-angiogenic factor (e.g., VEGF), buffers, media, pharmaceutically acceptable excipients, and instructions for its use. In certain embodiments, the contents of the kit are sterilized and packaged in a convenient form for use in a clinical setting.

In certain embodiments, the invention provides vectors for delivering one or more pro-angiogenic factors. These vectors may be viral, modified viral, or non-viral vectors encoding pro-angiogenic factors such as VEGF. FGF-1, FGF-2, angiogenin, TGFα, TGFβ, VPF, IL-3, IL-8, PDEGF, G-CSF, scatter factor, PDGF, etc. In certain embodiments, the gene encoding the pro-angiogenic factor in the vector is the same as the one found in Nature. In other embodiments, the gene encoding the pro-angiogenic factor is engineered by man. Expression of the gene encoding the angiogenic factor may be controlled by a stimulus such as hypoxia, pH, cell stress, etc. In certain embodiments, the vector provides for expression of the pro-angiogenic factor in mammalian, preferably human, cells. In certain embodiments, the vector provides for expression of the pro-angiogenic factor in cells found in the heart such as endothelial cells, endocardial cells, myocardial cells, epicardial cells, blood cells, myoblasts, fibroblasts, nerve cells, etc. In certain particular embodiments, the vector is a modified virus, e.g., modified adenovirus.

In another aspect, the invention provides cells which have been genetically engineered to express at least one pro-angiogenic factor (e.g., VEGF). These cells may be any type of cell; however, skeletal myobalsts, cardiomyocytes, fetal cardiomyocytes, embryonic stem cells, mesenchymal stem cells, or adult bone marrow-derived cells are preferred. Typically, the cells are mammalian cells, preferaly human cells. The cells may be permanently or temporarily modified to express the pro-angiogenic factor(s). In certain embodiments, the gene or construct encoding the pro-angiogenic factor is integrated into the genome of the cell. In other embodiments, the gene is not part of the chromosomes of the cell. The gene may be engineered by the hand of man. As described above for the vectors of the invention, the cells may constitutively express the pro-angiogenic factor or expression of the pro-angiogenic factor may be induced by such stimuli as hypoxia or low pH.

### BRIEF DESCRIPTION OF THE DRAWING

FIG. 1 shows stained sections six weeks after autologous skeletal myoblast (ASM) injection in sheep with ischemic heart failure (HF), composite Trichrome (A) and skeletal muscle specific myosin heavy chain (B, MY-32, purple staining) staining demonstrates extensive patches of ASM-derived skeletal muscle fibers engrafted in areas of myocardial scar. In panels C and D, at higher magnification from panel A (arrow), skeletal fibers were seen aligned with each other and further organized into myofibril bundles (Panels C and D). ASM-derived skeletal muscle aligned with remaining cardiac myocytes (Panel E, ‘c’) and with neighboring skeletal myofibers confirmed with staining for MY-32 (F). Scale bars in panels B, D and F are 2 mm, 0.5 mm, and 0.2 mm, respectively.

FIG. 2 shows viable muscle within an area of myocardial fibrosis and scar as seen with Trichrome staining (A). Staining with MY-32 (B) confirmed that ASM-derived skeletal muscle engrafted in close proximity and aligned with remaining cardiac myocytes (‘c’), but the ASM-derived skeletal muscle did not selectively stain for cardiac-specific tropinin-I (C). At higher magnification from the same area (C, arrow), ASM-derived skeletal myocytes do not stain for connexin43 (D), an integral component of cardiac cell gap junctions, despite very close apposition to remaining cardiac myocytes (‘c’). Scale bars in panels A and D are 0.2 mm and 0.1 mm, respectively.

FIG. 3 represents left ventricular volume (LVV) and pressure (LVP) tracings from a single sheep before and after microembolization (top and middle panels); highlight changes in the ESPVR (middle) and the PRSW (bottom, squares) with or without ASM transplantation (bottom panel, circles) after microembolization. Though ASM transplantation did not improve cardiac function (slope) after week 1 (∘ and □), transplantation did prevent a rightward shift in the PRSW seen in the HF control animal at week six (▪ and ●).

FIG. 4 demonstrates that left ventricular dilatation (ESVI, top panel) and an increase in mid papillary short-axis length (SA, middle panel) were attenuated after ASM injection (N=5, open bars) as compared to heart failure controls (N=6, shaded bars). Left ventricular long-axis length (LA, bottom panel) was not different between groups. All animals, including HF controls (“none”), were used to evaluate the relationship of ASM-derived myocyte survival (log) to that of LV remodeling (inset each panel, N=11). Animals with the highest ASM-derived myocyte survival demonstrated the greatest attenuation, particularly in LV short-axis dilatation. Correlative statistics are presented for each relationship.

FIG. 5 shows trichrome stains (A & C) which demonstrate viable myocytes in alignment with other skeletal myofibers and also with native cardiac myofibers (arrow shows axis in both C & D). Again MY-32 staining (fast mysoin heavy chain) in B compared to A confirms skeletal muscle.

FIG. 6 shows representative sheep hearts before (A) and after (B) left circumflex coronary artery microembolization.

FIG. 7 is a schematic of the left ventricle demonstrating placement of 3 sets of sonomicrometry crystals used for chronic, simultaneous and real-time measurement of short-axis (SA), long axis (LA), and ventricular segment length (SL). SA and LA dimensions are used to derive left ventricular volume in real-time allowing for pressure-volume analysis from pressure volume loops.

FIG. 8 shows the temporal relationship of decrease in LVEF (line) and concomitant increase in left ventricular end-systolic volume index (bars) in sheep from baseline to week 6 of heart failure (N=5). Bracket shows significance p<0.05 versus baseline.

FIG. 9 shows representative pressure-volume loops during inferior vena cava occlusion. End-systolic (ESPVR) and end-diastolic (EDPVR) pressure volume relationships are shown (left). Preload recruitable stroke work (PRSW) plot is generated during the same occlusion.

FIG. 10 shows attenuation of LV dilatation occurred in a myoblast survival-dependent fashion. Those animals with the highest myoblast survival (ASM-high, N=2) demonstrated the greatest benefit (bracket, top panel) in LV dilatation at week 6 as compared to both CHF control and sheep with lower myoblast survival (ASM-low, N=3). CHF+ASM-high demonstrated no increase in the short-axis diameter at week 6 (lower panels). No differences were found in long-axis dilatation between groups at week 6 (lower panels).

### DEFINITIONS

An agent is any chemical compound or composition of chemical compounds. These chemical compounds may include biological molecules such as proteins, peptides, polynucleotides (DNA, RNA, RNAi), lipid, sugars, etc.), natural products, small molecules, polymers, organometallic complexes, metals, etc. In certain embodiments, the agent is a small molecule. In other embodiments, the agent is a nucleic acid or polynucleotide. In yet other embodiments, the agent is a peptide or protein. In other embodiments, the agent is a non-polymeric, non-oligomeric chemical compound. In other embodiments, the agent is a vector such as a modified viral vector expressing a pro-angiogenic factor. In certain embodiments, the agent is a pharmaceutical approved for use in humans by the FDA. In certain embodiments, the agent is a cell, for example, a cell expressing a pro-angiogenic peptide or protein.

Angiogenesis refers to the formation of new blood vessels (e.g., capillaries). Particularly as used in the present invention, angiogenesis refers the formation of new blood vessels in heart tissue into which cells are or will be implanted. In certain embodiments, the cells, when implanted into an ischemic zone, enhance angiogenesis. Angiogenesis can occur, e.g. as a result of the act of transplanting the cells, as a result of ischemia, and/or as a result of administering a pro-angiogenic factor such as VEGF.

Cardiac damage or disorder characterized by insufficient cardiac function includes any impairment or absence of a normal cardiac function or presence of an abnormal cardiac function. Abnormal cardiac function can be the result of disease, injury, and/or aging. As used herein, abnormal cardiac function includes morphological and/or functional abnormality of a cardiomyocyte, a population of cardiomyocytes, or the heart itself. Non-limiting examples of morphological and functional abnormalities include physical deterioration and/or death of cardiomyocytes, abnormal growth patterns of cardiomyocytes, abnormalities in the physical connection between cardiomyocytes, under- or over-production of a substance or substances by cardiomyocytes, failure of cardiomyocytes to produce a substance or substances which they normally produce, and transmission of electrical impulses in abnormal patterns or at abnormal times. Abnormalities at a more gross level include dyskinesis, reduced ejection fraction, changes as observed by echocardiography (e.g., dilatation), changes in EKG, changes in exercise tolerance, reduced capillary perfusion, and changes as observed by angiography. Abnormal cardiac function is seen with many disorders including, for example, ischemic heart disease, e.g., angina pectoris, myocardial infarction, chronic ischemic heart disease, hypertensive heart disease, pulmonary heart disease (cor pulmonale), valvular heart disease, e.g., rheumatic fever, mitral valve prolapse, calcification of mitral annulus, carcinoid heart disease, infective endocarditis, congenital heart disease, myocardial disease, e.g., myocarditis, dilated cardiomyopathy, hypertensive cardiomyopathy, cardiac disorders which result in congestive heart failure, and tumors of the heart, e.g., primary sarcomas and secondary tumors.

Derived from refers to a cell that is obtained from a sample or subject or is the progeny or descendant of a cell that was obtained from the sample or subject. A cell that is derived from a cell line is a member of that cell line or is the progeny or descendant of a cell that is a member of that cell line. A cell derived from an organ, tissue, individual, cell line, etc., may be modified in vitro after it is obtained. For example, the cell may be engineered to express a gene of interest. Such a cell is still considered to be derived from the original source.

Engrafts are the incorporation of transplanted muscle cells or muscle cell compositions into heart tissue with or without the direct attachment of the transplanted cell to a cell in the recipient heart (e.g., by the formation desmosomes or gap junctions) such that the cells enhance cardiac function, e.g., by increasing cardiac output, or prevent or slow decreases in cardiac function.

GATA transcription factor includes members of the GATA family of zinc finger transcription factors. GATA transcription factors play important roles in the development of several mesodermally derived cell lineages. Preferably, GATA transcription factors include GATA-4 and/or GATA-6. The GATA-6 and GATA-4 proteins share high-level amino acid sequence identity over a proline-rich region at the amino terminus of the protein that is not conserved in other GATA family members.

Cell survivial, myoblast survival, or fibroblast survival within the heart refers to any of the following and combinations thereof: (1) survival of the cells, myoblasts, or fibroblasts themselves; (2) survival of cells into which the cells, myoblasts, or fibroblasts differentiate; (3) survival of progeny of the cells, myoblasts, or fibroblasts; and (4) survival of fusion products (i.e., cells with which the cells, myoblasts, or fibroblasts fuse).

Myocardial ischemia refers to a lack of oxygen flow to the heart which results in myocardial ischemic damage. As used herein, the phrase myocardial ischemic damage includes damage caused by reduced blood flow to the myocardium. Non-limiting examples of causes of myocardial ischemia and myocardial ischemic damage include: decreased aortic diastolic pressure, increased intraventricular pressure and myocardial contraction, coronary artery stenosis (e.g., coronary ligation, fixed coronary stenosis, acute plaque change (e.g., rupture, hemorrhage), coronary artery thrombosis, vasoconstriction), aortic valve stenosis and regurgitation, and increased right atrial pressure. Non-limiting examples of adverse effects of myocardial ischemia and myocardial ischemic damage include: myocyte damage (e.g., myocyte cell loss, myocyte hypertrophy, myocyte cellular hyperplasia), angina (e.g., stable angina, variant angina, unstable angina, sudden cardiac death), myocardial infarction, and congestive heart failure. Damage due to myocardial ischemia may be acute or chronic, and consequences may include scar formation, cardiac remodeling, cardiac hypertrophy, wall thinning, dilatation, and associated functional changes. The existence and etiology of acute or chronic myocardial damage and/or myocardial ischemia may be diagnosed using any of a variety of methods and techniques well known in the art including, e.g., non-invasive imaging (e.g., MRI, echocardiography), angiography, stress testing, assays for cardiac-specific proteins such as cardiac troponin, and clinical symptoms. These methods and techniques as well as other appropriate techniques may be used to determine which subjects are suitable candidates for the treatment methods described herein.

A peptide or protein comprises a string of at least three amino acids linked together by peptide (amide) bonds. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc.

Polynucleotide or oligonucleotide refers to a polymer of at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5 propynyl-cytidine, C5-bromouridine, C5-fluorouridine, C5-iodouridine, C5-propynyl-uridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

Small molecule refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. In certain other preferred embodiments, natural-product-like small molecules are utilized.

Skeletal myoblasts and skeletal myoblast cells refer to precursors of myotubes and skeletal muscle fibers. The term skeletal myoblasts also includes satellite cells, mononucleate cells found in close contact with muscle fibers in skeletal muscle. Satellite cells lie near the basal lamina of skeletal muscle myofibers and can differentiate into myofibers. As discussed herein, preferred compositions comprising skeletal myoblasts lack detectable myotubes and muscle fibers. The term cardiomyocyte includes a muscle cell which is derived from cardiac muscle. Such cells have one nucleus and are, when present in the heart, joined by intercalated disc structures.

Stem cell refers to any pluripotent cell that under the proper conditions will give rise to a more differentiated cell. Stem cells which may be used in accordance with the present invention include mesenchymal, muscle, cardiac muscle, skeletal muscle, fetal stem cells, neural stem cells, endothelial stem cells, pluripotent stem cells, hematopoietic stem cells, bone marrow stem cells, and embryonic stem cells. Stem cells useful in the present invention may give rise to cardiac myocytes or other cells normally found in the heart (e.g., mesenchymal stem cells). Stem cells can also be characterized by their ability (1) to be self-renewing and (2) to give rise to further differentiated cells. This has been referred to as the kinetic definition.

Treating as used herein refers to reducing or alleviating at least one adverse effect or symptom of myocardial damage or dysfunction. In particular, the term applies to treatment of a disorder characterized by myocardial ischemia, myocardial ischemic damage, cardiac damage, or insufficient cardiac function. Adverse effects or symptoms of cardiac disorders are numerous and well-characterized. Non-limiting examples of adverse effects or symptoms of cardiac disorders include: dyspnea, chest pain, palpitations, dizziness, syncope, edema, cyanosis, pallor, fatigue, and death. For additional examples of adverse effects or symptoms of a wide variety of cardiac disorders, see Robbins et al. (1984) Pathological Basis of Disease (W.B. Saunders Company, Philadelphia) 547-609; Schroeder et al., eds. (1992) Current Medical Diagnosis & Treatment (Appleton & Lange, Connecticut) 257-356.

Vector as used herein refers to any nucleic acid or nucleic acid-containing entity, wherein the nucleic acid encodes a protein to be expressed. The vector may be any entity for transferring a nucleic acid such a a plasmid, cosmid, artificial chromosome, natural chromosome, virus, or modified virus. In certain preferred embodiments of the invention, the vector encodes at least one pro-angiogenic factor.

### DETAILED DESCRIPTION OF PREFERRED EMBODIMENTS

The present invention provides a system for treating a patient suffering from heart disease. The treatment system is useful for treating any type of heart disease including cardiomyopathy, hypertrophic cardiomyopathy, dilated cardiomyopathy, atherosclerosis, coronary artery disease, ischemic heart disease, myocarditis, viral infection, wounds, hypertensive heart disease, valvular disease, congenital heart disease, myocardial infarction, congestive heart failure, arrhythmias, etc. The inventive system is particularly useful in treating diseases of the heart involving damage to cardiac tissue such as a loss of contractility (e.g., as might be demonstrated by a decreased ejection fraction). The inventive system is also not limited to the treatment of human but can be used in the treatment of any animal including domesticated animals or pets. The inventive system may also be used in experimental animals such as mice, rats, dogs, pigs, sheep, and primates (e.g., apes, chimpanzees, monkeys). The inventive system provides for the treatment of animals suffering from heart disease, particularly diseases involving the loss of contractility in the heart, ischemic heart disease, or diseases resulting in remodeling of the heart.

Patients to be treated using the inventive system may be selected based on various criteria as would be appreciated by a treating physician. These criteria may include disease of the patient, age of patient, prognosis, lifestyle of patient, EKG, echocardiogram, ejection fraction, stroke volume, left ventricular end-systolic index (LVESI), cardiac output, blood pressure, laboratory values such as cardiac enzymes, clinical signs and symptoms, exercise tolerance testing, position on transplant list, etc. The treating clinician will evaluate these criteria and determine whether the patient is suitable for treatment using the inventive system. In certain embodiments, the patient has ischemic heart disease. In certain embodiments, the patient suffers from diffuse coronary artery disease. In other embodiments, the patient has suffered a myocardial infarction. In yet other embodiments, the patient has undergone an invasive procedure such as angioplasty or coronary artery bypass grafting. The patient may be selected for treatment to prevent or reduce cardiac remodeling after a myocardial infarction or other ischemic disease. The patient may be selected for treatment to increase cardiac output.

In certain embodiments, the inventive system is combined with other treatments. For example, the inventive system may be combined with the use of drug therapy. The inventive system may also be used in conjunction with cardiac devices such as left ventricular assist devices, balloon pumps, or pacemakers. In yet other embodiments, the inventive treatment system is used to improve cardiac function while the patient is waiting for a heart transplant. In certain embodiments, the treatment system provides a bridge to recovery.

In one aspect, the inventive system includes both methods and compositions for implanting cells into the heart of the patient in combination with treatment using at least one pro-angiogenic factor. Without wishing to be bound by any particular theory, it is hypothesized that by combining cellular cardiomyoplasty with the administration of a pro-angiogenic factor, the cells being transplanted have a better survival rate when compared to treatment with cellular cardiomyoplasty alone. The treatment with pro-angiogenic factor(s) leads to the development of new blood vessels in the ischemic, damaged, or injured area of the heart which will receive the implanted cells. For example, the inventors have shown that an adenoviral vector engineered to express VEGF can be used to induce revascularization in damaged myocardium. These results combined with cellular cardiomyoplasty may lead to better survival and engraftment of the implanted cells. The oxygen and other nutrients provided by the new blood vessel growth leads to a better survival rate in the implanted cells. The cells may also be better able to differentiate and/or integrate themselves into the myocardium of the heart, forming the syncitium of cells needed for coordinated contraction of the myocardium of the patient's heart. In certain embodiments, at least 10%, 20%, 30%, 50%, 60%, 70%, 80%, or 90% of the transplanted cells remain three weeks, six months, or 1 year after cellular myoplasty. In certain embodiments, at least 50% of the transplanted cells remain three weeks after transplantation. Preferably, these cells are integrated into the existing myocardium of the patient's heart. In certain embodiments, the implanted cells fused or form gap junction with the existing myocytes of the patient's heart. The cells may become part of the synctium of cells in the myocardium.

The inventive system includes two phases. The first phase involves promoting angiogenesis in heart tissue of the patient. In certain embodiments, the heart tissue being treated is ischemic (i.e., lacking an adequate oxygen or blood supply). In certain embodiments, the heart tissue is damaged or injured. The second phase includes the transplantation of cells into the heart (i.e., cellular cardiomyoplasty). The first phase does not need to occur before the second; however, in certain embodiments, angiogenesis is promoted before the cells are implanted. In certain embodiments, the first and second phase are performed concurrently. The different phases may also be repeated independently or in combination to improve the results of the inventive therapy. For example, the first phase of promoting angiogenesis may be repeated several times before cells are implanted. In other situation, cells may be implanted multiple times to increase the number of engrafted cells in the patient's heart. The phases of the inventive system may be repeated until a desired effect has been achieved, e.g., cardiac output, ejection fraction, stabilization of cardiac remodeling, etc.

Promoting Angiogenesis

In the inventive system, cells are administered into the heart of a patient after or concomitantly with the administration of at least one pro-angiogenic factor. In certain embodiments, the step of promoting angiogenesis is performed before the cells are administered to the heart. Particularly, the pro-angiogenesis therapy is begun days to weeks to months before the cells are administered. The timing is determined empirically by the treating physician considering such factors as the disease being treated, the extent of the disease, the condition of the patient, the extent of ischemia, the condition of the site of transplantation, how the pro-angiogenic factor(s) is/are administered, when pro-angiogenic factor(s) is/are being administered, which type(s) of cell is/are to be implanted, etc. In certain embodiments, the duration of time between administration of an angiogenesis factor and administration of cells may range from 3 days to 8 weeks. In certain embodiments, the range is from 1 week to 6 weeks, and in still other embodiments, the range is from 2 weeks to 5 weeks. In yet other embodiments, the cells are administered approximately 3-4 weeks after the angiogenesis therapy is begun. In certain embodiments, the step of administering a pro-angiogenic factor may be repeated before, after, or during the implantation of cells.

The angiogenesis therapy is generally designed to improve blood flow in the damaged or diseased region of the heart to provide a better substrate on which the implanted cells can grow, divide, and/or engraft themselves. In certain embodiments, a region of the patient's heart is ischemic. Any agent known to induce angiogenesis may be used in the angiogenesis promoting step. The agent may be a protein, a peptide, a polynucleotide, an aptamer, a virus, a small molecule, a chemical compound, a cell, etc. In certain embodiments, the agent is a pro-angiogenic protein/peptide such as vascular endothelial growth factor (VEGF). Other examples of protein/peptide-based pro-angiogenic factors include angiogenin, growth factors, hypoxia-inducible factor-1 (HIF-1), epidermal growth factor (EGF), bFGF, angiopoietin, acidic fibroblast growth factor (FGF-1), basic fibroblast growth factor (FGF-2), platelet-derived growth factor, angiogenic factor, transforming growth factor-alpha (TGF-α), transforming growth factor-beta (TGF-β), vascular permeability factor (VPF), tumor necrosis factor alpha (TNF-α), interleukin-3 (IL-3), interleukin-8 (IL-8), platelet-derived endothelial growth factor (PD-EGF), granulocyte colony stimulating factor (G-CSF), hepatocyte growth factor (HGF), scatter factor (SF), pleitrophin, proliferin, follistatin, placental growth factor (PIGF), midkine, platelet-derived growth factor-BB (PDGF), and fractalkine. In certain embodiments, combinations of the above pro-angiogenic factors are used. Derviatives or modified versions of these pro-aniogenic factors are also useful in the invention. These modified version are typically 75%, 80%, 90%, 95%, 98%, 99%, or 100% identical to the wild protein or peptide. In certain embodiments, these modified versions show at least 50%, 75%, 80%, or 90% overall identity and share recognized or conserved sequence elements. Modified versions, fusions, or derivatives also include forms in which at least conserved or characteristic sequence elements have been placed in non-natural environments. In certain embodiments, the modified versions or derivatives have enough of the sequence of a pro-angiogenic factor to have the substantially the same activity as the naturally occurring factor. Any other pro-angiogenic factors known or discovered in the future may be used to promote angiogenesis in the inventive treatment system. In certain embodiments, a combination of angiogenic factors is used to promote angiogenesis in the heart of the patient.

In other embodiments, the agent is delivered via a polynucleotide which encodes and expresses a pro-angiogenic protein/peptide such as VEGF or any of the other pro-angiogenic factors listed above. In particular, the polynucleotide contains a gene that encodes a pro-angiogenic protein/peptide. In certain embodiments, the polynucleotide is DNA based. In other embodiments, the polynucleotide is RNA based. In certain embodiments, the polynucleotide is a modified DNA molecule. The vector may be a polynucleotide designed to integrate into the genome of a cell. In other embodiments, the vector does not integrate into the genome of the cells of the patient. In certain embodiments, the polynucleotide is a plasmid, a cosmid, a virus (e.g., adenovirus or adeno-associated virus), an artificial chromosome, or a genetically engineered chromosome. The vector may contain other nucleotide sequences such as promoters, elements for controlling gene expression, transcription stop sequences, ribosomal binding sequences, splicing control elements, selection markers, housekeeping genes, origin of replication, etc. In certain embodiments, the vector includes an entire gene or a portion of the gene encoding a pro-angiogenic factor. In certain embodiments, the vector encodes VEGF or a variant of VEGF (e.g., VEGF₁₂₁). In other embodiments, the vector may include a gene modified by the hand of man.

In certain embodiments, the gene encoding the angiogenic factor is constitutively expressed, e.g., under control of a cytomegalovirus (CMV) promoter. In other embodiments, expression of the gene is induced by a stimulus such as hypoxia, lack of nutrients, increase in carbon dioxide, change in pH, cell stress, etc. In other embodiments, the vector is contructected such that the gene is expressed in certain cell types such as mammalian cells, human cells, cardiomyocytes, endothelial cells, fibroblasts, muscle cells, skeletal myoblasts, myocardial cells, epicardial cells, fat cells, blood cells, etc. In certain embodiments, the cell is a mesenchymal stem cell, endothelial stem cell, or a myoblast. Other cells useful in promoting angiogenesis include bone marrow derived stem cells, hematopoietic stem cells, embryonic stem cells , cord blood cells, primordial germ cells, neural stem cells, and pluripotent stem cells. In certain embodiments, the cells is a stem cell. Preferably, the type of cells infected are found in the heart of the patient. In certain embodiments, the vector is constructed such that only a particular type of cell is transfectd.

In certain embodiments, the pro-agiogenic factor is delivered by a cell that is implanted or otherwise administered to the heart. Preferably, the cell to be implanted is genetically engineered to express at least one pro-angiogenic factor. In other embodiments, the cell may naturally express a pro-angiogenic factor. In certain particular embodiments, the cell secretes a pro-angiogenic factor. When the cell is engineered to express a pro-angiogenic factor, it typically contains a construct such as those described above for use in polynucleotide vectors. In certain embodiments, the genome of the cell is altered by inserting a construct engineered to express the pro-angiogenic factor. In certain other embodiments, the polynucleotide vectors described above may be used to transfect cells which are then implanted into the heart. In certain embodiments, the cells are the same type of cells to be implanted in cellular cardiomyoplasty. For example, useful cells are typically skeletal myobalsts, cardiomyocytes, fetal cardiomyocyes, embryonic stem cells, mesenchymal stem cells, or adult bone marrow-derived cells, and combinations thereof, optionally also including fibroblasts. The cells may also be fibroblasts, muscle cells, blood cells (e.g., white blood cells), endothelial cells, stem cells, progenitor cells, bone marrow cells, etc. The cells preferably are autologous cells so that no adverse reaction to the cells is caused by the implantation of the cells into the patient's body; however, cells may de derived from a relative or matched donor.

In other embodiments, the agent is a small molecule that is known to promote angiogenesis. For example, the small molecule may be one that induces angiogenesis pathways in endothelial cells, fibroblasts, stem cells, etc. The small molcule may mimic the three-dimensional structure of a pro-angiogenic peptide or protein. For example, the small molecule may bind and stimulate the receptor for VEGF. The structure of pro-angiogenic peptides or proteins as determined by x-ray crystallography, NMR studies, or other techniques may be useful in designing pro-angiogenic small molecules. Preferably the small molecule is FDA approved for use in humans. An example of a pro-angiogenic small molecule is bovine retinal angiogenesis factor. The administration of the small molecule (i.e., dosage, route, timing, etc.) will depend on the agent being delivered, the pharmacokinetics of the agent being delivered, the status of the patient, the degree of ischemia, etc. as would be appreciated by one of skill in this art.

The angiogenesis agent is typically delivered to the heart of the patient. In certain embodiments, the agent is delivered to an injured area of the heart, for example, an area of the heart that has suffered injury due to ischemia. The injured area of the heart may also be caused by an infection (e.g., viral, bacterial, or parasitic), by a chemical compound, iatrogenically, or any other means. In other embodiments, the agent is delivered to the border zone between injured and non-injured areas of the heart. In still other embodiments, the agent is delivered to a healthly area of the patient's heart. In certain embodiments, the agent is delivered to the heart as a whole without regard to injured or non-injured areas. The agent may be delivered intravenously, intraarterially, parenterally, intramuscularly, etc. In certain embodiments, the agent is delivered via a catheter to the heart of the patient, particularly the injured area. In other emdodiments, the agent is delivered intramuscularly into the heart of the patient during surgery. The agent may also be delivered into the heart of the patient using radiographic guidance of a needle, catheter, or other drug delivery device. In other embodiments, the agent is delivered systemically in a form designed to target the heart or a particular area of the heart. For example, the agent may be encapsulated in a polymeric matrix which includes a targeting means such as an antibody directed to cell surface molecule(s) found on cells of the heart (e.g., myocardial cells, endothelial cells). In other embodiments, a drug delivery device is implanted in the heart to provide time release of the pro-angiogenic factor(s). In other embodiments, the agent is a virus which targets cells of the heart, particularly myocardial cells or endothelial cells. The virus may be genetically engineered to target cells of the heart.

As described above, the administration of the angiogenesis factor may be repeated. In certain embodiments, the administration is repeated before the cells are transplanted in order to increase angiogenesis in the heart. The administration may also be repeated after the cells are implanted to continue to promote angiogenesis. The administration may be repeated, for example, every day, every other day, every third day, every fifth day, every week, every two weeks, every three weeks, or every four weeks, or less frequently. The precise regimen for administering the pro-angiogenic factor is determined by the treating physician taking into account such factors as the patient's health, the angiogenesis agent being delivered, how the agent is administered, the disease being treated in the patient, the severity of the disease, etc.

Administration of Cells

The second part of the inventive system involves the transplantation of cells into the diseased area of the heart, also known as cellular cardiomyoplasty. Cells are implanted into a diseased or injured area of the heart to improve cardiac function. Cells that are useful in the inventive system include cells that can proliferate and engraft themselves into the existing myocardium of the patient. Cells found to be particularly useful in the inventive system include myoblasts (e.g., skeletal myoblasts), mesenchymal stem cells, fetal cardiomyocytes, embryonic stem cells, and bone marrow stem cells. In certain embodiments, skeletal myoblasts are used in the inventive system. For further discussion of skeletal myoblasts useful in the inventive system, please see U.S. Patent Applications Ser. No. 60/145,894, filed Jul. 23, 1999; U.S. Ser. No. 09/624,885, filed Jul. 24, 2000; and U.S. Ser. No. 10/105,035, filed Mar. 21, 2002; each of which is incorporated herein by reference. In certain embodiments, cardiomyocytes are used in the inventive system (see, for example, U.S. Pat. Nos. 6,673,604; 6,491,912; 5,919,449; published U.S. Patent Application 2003/0232431; 2003/0022367; 2001/0053354; each of which is incorporated herein by reference). In certain embodiments, the cells administered are a 50%, 60%, 70%, 80%, 90%, 95%, 98%, 99% pure population of cells. As discussed in the above referenced applications, the purity of the skeletal myoblast or other cell population may be obtained by culturing cells from a muscle biopsy under certain conditions with 5-20 doublings, preferably 10-15 doublings, or more preferably 11-12 doublings (see Jain et al. Circulation 103:1920-1927, 2000; incorporated herein by reference). In certain embodiments, the cells are not cultured. In other embodiments, the cells are minimally cultured. For example, the cells may be left on a cell culture plate for a few days followed by removal of non-adherent cells. In certain embodiments, all or a substantial portion of the cells have not undergone cell division before they are administered. In other embodiments, the cells have undergone 1-2 doublings, 3-4 doublings, or 5-10 doublings. The purity of the skeletal myoblasts may be tested by the presence of the CD56 marker or other markers on the cells. In other embodiments, the cells are stem cells (e.g., embryonic stem cells, fetal stem cells, adult-derived stem cells, etc.).

In certain embodiments, the cells are mesenchymal stem cells or cells derived from mesenchymal stem cells. In certain embodiments, cells are treated in a manner which causes them to acquire stem cell qualities. Typically, the cells are immature or undifferentiated, allowing them to differentiate into myocytes after implantation. In certain embodiments, the mesenchymal stem cells are obtained from the bone marrow of the patient. The mesenchymal stem cells may be co-cultured with cardiomyocytes such as fetal cardiomyocytes. The culturing with fetal cardiomyocytes helps to obtain cells that have pre-differentiated towards a cardiac myocyte phenotype. In certain embodiments, the stem cells may fuse with the cardiomyocytes. In other embodiments, this fusion is to be avoided. In certain embodiments, the cells are not cultured at all or are minimally cultured as described above.

The cells used in the inventive system can be obtained from any source. However, the cells are typically harvested from the patient so that there are no rejection issues (i.e., autologous transplantation). The cells, for example, may be harvested from the muscle of the patients, from the bone marrow, from the blood, from the fetal cord blood of the patient, etc. Besides autologous transplantation, the cells may be obtained from a relative, an MHC-matched donor, a donor of the same blood type, or any donor of the same species. In certain embodiments, cross-species donation of cells is used (i.e., xenogeneic transplantation). As would be appreciated by one of skill in this art, immunosuppression may be required if the donor cells are not from the patient or a related donor. The cells may also be treated or modified to reduce their immunogenicity. For example, the MHC class I molecules on the cells may be masked or modified to limit their immunogenicity.

In general, at least about 50%, 60%, 70%, 80%, 90%, 95%, 98%, or 99% of cells in a population should be viable (as determined by such methods as Trypan Blue exclusion) in order for the population to be useful in accordance with the present invention. Typically, the cell viability immediately before administration is greater than 90%, 95%, or 98%. The number of cells administered may range from 1×10⁴ to 1×10¹⁰ cells, or 1×10⁵ to 1×10⁹ cells, or 1×10⁶ to 1×10⁸ cells, or 1×10⁸ to 1×10⁹ cells. The cells may all be injected at one site or multiple sites. The number of cells administered will depend on the extent of damaged cardiac tissue. The cells are typically injected into the myocardium between the endocardium and epicardium over a 1-5 cm distance.

The cells used in the invention may also be genetically engineered. The cells may be engineered using any techniques known in the art. For example, the genomes of the cells may be altered permanently, or the cells may be altered to express a gene only transiently. In certain embodiments, the cells are genetically engineered to produce a pro-angiogenic peptide or protein as discussed above. In certain embodiments, the implantation of cells engineered to express at least one pro-angiogenic factor constitutes both the administration of a pro-angiogenic agent and implantation of cells. The angiogenic peptide/protein may be expressed constitutively in the transplanted cells, or it may be expressed upon a certain stimulus. Certain stimuli that may control gene expression include hypoxia, lack of nutrients, presence of growth factors, change in pH, build up of waste products, cell stress, etc. In certain embodiments, the transplanted cells of the invention may express an anti-apoptotic gene. In other embodiments, the cells express or can be induced to express a gene to increase proliferation such as a growth factor (e.g., basic fibroblast growth factor (bFGF)). In other embodiments, a cardiac cell phenotype is promoted in the cells by expressing a cardiac cell gene product in the cell. For example, the GATA transcription (e.g., GATA4, GATA6) may be expressed in the cells in order to promote the cardiac cell phenotype.

The cells are delivered into the injured tissue using any technique known in the art. The cells may be delivered during heart surgery. Alternatively or additionally, the cells may be delivered via a catheter. The cells are typically injected into the injured tissue using a syringe and needle. In certain embodiments, a side port needle is used to inject the cells into the tissue (see U.S. Patent Application Ser. No. 60/401,449, filed Aug. 6, 2002, and U.S. Ser. No. 10/635,212, published as US 2004/0191225, filed Aug. 6, 2003; each of which is incorporated herein by reference).

Due to the force of the contracting heart, it may be necessary to take steps to limit the number of cells that escape from the injection site in the myocardium. For example, pressure can be applied at the injection site for seconds to minutes after the needle has been removed. Alternatively or additionally, a viscosity enhancing agent, such as a matrix may be utilized, e.g., being combined with the cells prior to injection. The matrix may be a biocompatible polymer (e.g., cellulose, protein, polyethylene glycol, sorbitol, poly(lactic-glycolic acid), etc.) or other excipient such as glycerol, carbohydrates, etc. In certain embodiments, the polymer is also biodegradable. In some embodiments, the polymer is a biomatrix (e.g., a protein, ECM protein). In some embodiments, the polymer is a biogel. In certain embodiments, the matrix is Cymetra. In certain other embodiments, the matrix is a decellularized dermal matrix, preferably a decellularized dermal matrix. The matrix may also be a basement membrance matrix. The matrix may be impreganated with pro-angiogenic factor in certain embodiments. The matrix can be selected to allow for delayed release (e.g., over time and/or in response to a signal or environmental trigger) of pro-angiogenesis factor. In other embodiments, a plug (e.g., a polymeric plug) or bandage (e.g., suture) may be applied over the injection site to prevent the efflux of injected cells.

The inventive method may be repeated as determined by a treating physician. Certain steps of the method may be repeated. For example, a pro-angiogenic factor may be administered repeatedly, or cells may be transplanted repeatedly, or both. The disease and condition of the patient may be used in determing the extent to which repeat therapy is warranted. As described above, the inventive method may be combined with more traditional treatments such angioplasty, coronary artery bypass graft, left ventricular assist device, drug therapy, stent placement, heart transplant, etc.

The inventive system is designed to improve the cardiac function of the patient, stabilize cardiac function, or limit the decrease in cardiac function. In certain embodiments, the inventive system may improve cardiac function by 1%, 2%, 5%, 10%, 20%, 30%, 40%, 50%, 75%, 100%, 200%, 300%, 400%, or 500% as measured by any number of parameters including ejection fraction, stroke volume, cardiac output, blood pressure, etc. These parameters may be measured by echocardiography, MRI, catheterization, EKG, blood pressure cuff, pulse oximeter, etc. In certain embodiments, the improvement in cardiac function may be measured by exercise tolerance test. In certain embodiments, the inventive system prevents the dilatation and/or weakening of the heart, especially after ischemic injury to the heart. In certain embodiments, the inventive system maintains the left ventricular end-systolic index at greater than 50 mL/m², at greater than 60 mL/m², or at greater than 70 mL/m². In certain embodiments, left ventricular dilatation is decreased or stabilized. In certain embodiments, mid-papillary short axis length is decreased. In certain embodiments, the inventive system is used to stabilize the patient before another treatment is performed such as heart transplantation.

The inventive system may be combined with other treatment modalities. These other treatments include medication (e.g., blood pressure medication, calcium channel blockers, digitalis, anti-arrhythmics, ACE inhibitors, anti-coagulants, immunosuppressants, pain relievers, vasodilators, etc.), angioplasty, stent placement, coronary artery bypass graft, cardiac assist device (e.g., left ventricular assist device, balloon pump), pacemaker placement, heart transplantation, etc. In certain embodiments, the inventive system provides a bridge to recover for a patient waiting to undergo heart transplantation.

Kits

The present invention also provides kits useful for the practice of the inventive system. The kit typically contains any combination of equipment, apparatus, pharmaceuticals, biologicals, reagents, etc. useful in the practice of the invention. The contents of the kit are conveniently packaged for a treating physician, nurse, or other medical personnel to use. The materials in the kit may also be packaged under sterile conditions. In certain embodiments, the kit may contain any or all of the following: cells, syringes, catheters, needles (e.g., side port needles), media, buffers, angiogenic factors, vectors for expressing angiogenic factors (e.g., VEGF or any other factor described above), adenoviral vectors, storage containers, vials, anesthetics, antiseptics, instructions, polynucleotides, bandages, pharmaceutically acceptable excipient for delivering cells, tissue culture plates, etc.

In certain embodiments, a kit is provided for harvesting skeletal myoblasts from the patient, purifying the cells, and expanding the cells. Such a kit may include any of the following: needles, syringes, buffers, cell culture media, serum, storage media, glycerol, cell culture dishes, instruction manual, and combinations thereof. The kit may also include material for purifying cells. The kit may also contain materials for detecting the purity of the resulting population (e.g., antibodies directed to a cell marker).

In another embodiment, a kit is provided for practicing the treatment method. The kit may include any of the following: needles, catheters, syringes, angiogenic factors, vectors for expressing angiogenic factors, pharmaceutically acceptable excipient for injecting cells, instruction manual, and combinations thereof. In certain embodiments, the kit includes a purified angiogenic factor such as VEGF. The factor may be supplied as a lyophilized powder.

The invention also provides other materials and reagents which may be included in the kits as described above. For example, the invention provides vectors and polynucleotides useful in the present invention. In certain embodiments, the vector is a genetically engineered adenovirus. In certain particular embodiments, the vector is a genetically engineered adenovirus which leads to the expression of VEGF in cell it infects. The vector may also include control sequences for controlling the expression of the angiogenic factor. For example, the expression of the angiogenic factor may be induced a lack of oxygen, change in pH, build-up of waste products, etc. The vector may also contain sequences for replicating and selecting the vector.

The invention also provides cells for the inventive system. Typically, these cells are myoblasts (e.g., skeletal myoblasts), fetal cardiomyocytes, embryonic stem cells, and bone marrow stem cells. In certain preferred embodiments, the cells are skeletal myoblasts. The cells may be genetically engineered. In particular, the cells may be genetically engineered to express an angiogenic factor. In other instances, the cells may express an anti-apoptotic gene to prevent the cells from undergoing apoptosis. In certain embodiments, the cells are purified away from other cells or from other components the cells are normally found with.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Correlation of Autologous Skeletal Myoblast Survival with Changes in Left Ventricular Remodeling in Dilated Ischemic Heart Failure

Introduction

Autologous skeletal myoblast (ASM) transplantation, or cardiomyoplasty, has been shown in multiple experimental studies to improve cardiac function after myocardial infarction (MI) (Chiu et al. “Cellular cardiomyoplasty: myocardial regeneration with satellite cell implantation” Ann. Thor. Surg. 60:12-18, 1995; Li et al. “Cardiomyocyte transplantation improves heart function” Ann. Thor. Surg. 62:654-61, 1996; Murry et al. “Skeletal myoblast transplantation for repair of myocardial necrosis” J Clin. Invest. 98:2512-23, 1996; Scorsin et al. “Comparison of effects of fetal cadiomyocyte and skeletal myoblast transplantation on postinfarction left ventricular function” J. Thor. Cardiovasc. Surg. 119:1169-75, 2000; Tambara et al. “Transplanted skeletal myoblasts can fully replace the infarcted myocardium when they survive in the host in large numbers” Circulation 108[suppl II]:II-259-63, 2003; Taylor et al. “Regenerating functional myocardium: improved performance after skeletal myoblast transplantation” Nat. Med. 4(8):929-33, 1998; Jain et al. “Cell therapy attenuated deleterious ventricular remodeling and improves cardiac performance after myocardial infarction” Circulation 103:1920-1927, 2000; each of which is incorporated herein by reference). Though the majority of studies have been performed in small animal models of MI, there is evidence of similar improvement in larger animal models (Ghostine et al. “Long-term efficacy of myoblast transplantation on regional structure and function after myocardial infarction” Circulation 106[suppl I]:I131-6, 2002; incorporated herein by reference) and in the first patient trials (Menasche et al. “Myoblast transplantation for heart failure” Lancet 357:279-80, 2001; Menasche et al. “Autologous skeletal myoblast transplantation for severe postinfarction left ventricular dysfunction” J. Am. Coll. Cardiol. 41:1078-83, 2003; Pagani et al. “Autologous skeletal myoblasts transplanted to ischemia-damaged myocardium in humans” J. Am. Coll. Cardiol. 41:879-888, 2003; Pouzet et al. “Factors affecting functional outcome after autologous skeletal myoblast transplantation” Ann. Thorac. Surg. 71:844-851, 2001; each of which is incorporated herein by reference). The mechanism behind such positive functional changes remains poorly understood given that developing and engrafted skeletal myoblasts are electro-mechanically isolated from their host myocardium (as evidenced by the lack of connexin43 and/or gap junctions (Scorsin et al. “Comparison of effects of fetal cadiomyocyte and skeletal myoblast transplantation on postinfarction left ventricular function” J. Thor. Cardiovasc. Surg. 119:1169-75, 2000; Ghostine et al. “Long-term efficacy of myoblast transplantation on regional structure and function after myocardial infarction” Circulation 106[suppl I]:I131-6, 2002; Menasche et al. “Myoblast transplantation for heart failure” Lancet 357:279-80, 2001; Menasche et al. “Autologous skeletal myoblast transplantation for severe postinfarction left ventricular dysfunction” J. Am. Coll. Cardiol. 41:1078-83, 2003; Pouzet et al. “Factors affecting functional outcome after autologous skeletal myoblast transplantation” Ann. Thorac. Surg. 71:844-851, 2001; each of which is incorporated herein by reference). Furthermore, clinical ASM cardiomyoplasty has been applied exclusively to patients with severe ischemic cardiomyopathy, and more importantly, it has always been performed as an adjunct to coronary revascularization and/or left ventricular assist devices (LVADs) (Pagani et al. “Autologous skeletal myoblasts transplanted to ischemia-damaged myocardium in humans” J. Am. Coll. Cardiol. 41:879-888, 2003; incorporated herein by reference). Because of these concomitant therapies, the improvements in indices of myocardial perfusion, viability and function may be difficult to attribute to ASM injection alone.

Additionally, growing experimental evidence suggest that the number of ASM cells transplanted and the functional/geometrical impacts are directly related (Tambara et al. “Transplanted skeletal myoblasts can fully replace the infarcted myocardium when they survive in the host in large numbers” Circulation 108[suppl II]:II-259-63, 2003; Pouzet et al. “Factors affecting functional outcome after autologous skeletal myoblast transplantation” Ann. Thorac. Surg. 71:844-851, 2001; each of which is incorporated herein by reference). For example, Tambara et al. (“Transplanted skeletal myoblasts can fully replace the infarcted myocardium when they survive in the host in large numbers” Circulation 108[suppl II]:II-259-63, 2003; incorporated herein by reference) using fetal-derived ASM in rats demonstrated that both cardiac function and remodeling were impacted in a dose dependent fashion. However, these benefits have not been demonstrated in ischemic dilated HF, where elevated wall stresses and altered myocardial mechanoenergetics could compromise ASM survival, differentiation, and ultimately functional efficacy. Thus, the aims of the present study were to evaluate LV remodeling and function after ASM transplantation into an animal model of end-stage ischemic HF (LVEF <35% and LV end-systolic volume >80 ml/m²). Furthermore the study also sought to evaluate the survival, differentiation and alignment of ASM injected into those same animals.

Materials and Methods

Ischemic Heart Failure Model

Experimental ischemic heart failure was created in sheep as Sabbah et al (“A canine model of chronic heart failure produced by multiple sequential coronary microembolizations” Am. J. Physiol. 260:H1379-84, 1991; incorporated herein by reference) described in dogs with minor modifications. Briefly, serial and selective left circumflex coronary artery (LCxA) microembolizations (2.9±0.4 injections per animal) were performed by injecting polystyrene beads (70-110 μm) weekly until the left ventricular ejection fraction (LVEF) was maintained at or below 35% for 2 consecutive weeks.

Experimental Groups

The HF control group of sheep (baseline) was instrumented 2 weeks prior to LCxA microembolizations and HF induction (HF control, N=6). The transplanted group of sheep had LCxA microembolization and HF induction prior to instrumentation and injection with ASM (HF+ASM, N=5). Studies were performed weekly for 6 weeks in awake and unsedated animals.

Chronic Instrumentation

All sheep were instrumented through a left thoracotomy; a left ventricular (LV) solid-state electronic pressure transducer (4.0 or 4.5 mm, Konigsberg, CA) was placed into LV at its apex and chronic, heparinized (1000 U/mL) fluid filled catheters (Tygon) were inserted for monitoring of aortic, LV, and right ventricular (RV) pressures. Six piezoelectric crystals (Sonometrics Inc., New London, Ontario Canada) were surgically placed in the LV endocardium at the mid papillary level (short axis, SA), at the LV base and apex (long-axis, LA) and in the mid myocardium of the posterolateral LV (segment length, SLpost). A 16 mm occluder (In Vivo Metrics. Healdsburg, Calif.) was positioned around the inferior vena cava (IVC). All catheters and cables were tunneled to positions between the animals'scapula.

Hemodynamic Measurements and Pressure Volume Analysis

Aortic, RV, and LV fluid-filled catheters were attached to calibrated Statham pressure transducers (Model: P23XL; Biggo-Spectramed, Ocknard, Calif.) and amplified (Gould, Valley, Ohio). The electronic LV pressure gauge was calibrated using the LV fluid-filled catheter. Pressures waveforms were collected (at 1 kHz) and analyzed by a 16-channel data acquisition and software system (IOX; EMKA Technologies, Falls Church, Va.).

Sonometric signals were analyzed for waveform cardiac-cycle dependent (end-diastolic and end-systolic) and independent (minimum, maximum, mean etc) parameters. Left ventricular volume (ml) was calculated in real-time using SA and LA dimensions and the following equation: SA²* LA* π/6)/1000. Left ventricular volume indices were calculated: LV volume* body surface area (ml/m²). Inferior vena cava occlusions were performed for the generation of PV relationships that were analyzed off-line with analysis software (IOX, EMKA).

Left ventricular work was estimated (Todaka et al. “Characterizing ventricular mechanics and energetics following repeated coronary microembolizations” Am. J. Physiol. 272:H186-94, 1997; Suga, “Ventricular Energetics” Physiol. Rev. 70:247-77, 1990; each of which is incorporated herein by reference) by calculating the pressure volume area (PVA). PVA was calculated from off-line ESPVR-derived data as the sum of the LV potential energy (PE_{LV}) and stroke work (SW_{LV}).
PE_{LV}=(½[V₀−LVESV]×LVESP)
PVA=SW_{LV}+PE_{LV}
V₀: volume of the LV at zero pressure (x-intercept of Eₑₛ), LVESV: LV end-systolic volume (mL) and LVESP: LV end-systolic pressure.
Skeletal Muscle Biopsy and Autologous Skeletal Myoblast Culture

Skeletal muscle biopsy (1-3 grams) was harvested from the left forelimb of sheep at the time of the first microembolization in HF+ASM sheep. The forelimb muscle was exposed and the biopsy taken using sharp dissection avoiding electrocautery and placed into a tube containing biopsy transport media and shipped to GenVec, Inc (Charlestown, Mass.) for ASM preparation and culture similar to that described by Jain et al. (Jain et al. “Cell therapy attenuated deleterious ventricular remodeling and improves cardiac performance after myocardial infarction” Circulation 103:1920-1927, 2000; incorporated herein by reference).

All cells were expanded for 1-12 doublings and cryopreserved prior to transplant. The myoblasts were thawed, formulated in Transplantation Media, and shipped for direct myocardial injection. Myoblast purity was measured by reactivity with anti-NCAM mAb (CD56-PE, Clone MY-31, BD Biosciences, San Diego, Calif.) and by the ability to fuse into multinucleated myotubes. Cell viability was determined by Trypan Blue exclusion. Myoblasts were loaded into tuberculin syringes (˜1.0×10⁸ cells/mL) and shipped at 4° C. At the time of transplant, cells were allowed to warm slowly to room temperature, resuspended by gentle agitation and injected without further manipulation. Autologous skeletal myoblasts were injected at multiple sites in the infarcted myocardium in proximity to segmental sonomicrometry crystals. To avoid inadvertent intravascular or intraventricular injection of cells, the injection needle was passed into the mid myocardium equidistant from the epi- and endocardial surfaces over a 3-4 cm distance, negative pressure was applied to the syringe and if no blood was returned the cells were injected as the needle was slowly withdrawn. Slight pressure was applied over the needle exit site for several seconds after injection to limit cell efflux from the needle track.

Histology

After six weeks of study, each animal was euthanized, the heart removed, and perfused with 10% buffered formalin. Tissue blocks were made from embolized myocardium receiving ASM injection. Hematoxylin and eosin and Trichrome stains were performed using standard methods.

Immunohistochemistry

Deparaffinized sections were stained immunohistochemically with an anti-myosin heavy chain antibody that does not react with cardiac muscle, alkaline phosphatase-conjugated MY-32 mAb (Sigma, St Louis, Mo.), to confirm the phenotype of the mature grafts. Sections were developed with BCIP-NBT (Zymed Lab Inc., San Fransisco, Calif.) and counter stained with nuclear red. Additionally stains for connexin-43 Ab (Chemicon, Temecula, Calif.), and cardiac specific troponin I (Chemicon) were performed.

Estimation of Myoblast Survival

The heart was cut into blocks approximately 2.5 cm×2.5 cm×3 mm in dimension and processed in paraffin. In some cases the whole block was sectioned (5 μm thickness), in other cases, only a portion of the tissue was sectioned. For performing quantitative cell counts, tissue sections were then immunostained for skeletal-specific myosin heavy chain (MY-32). Cell viability at 6 weeks was assumed based on the initiation of myosin heavy chain expression (Havenith et al. “Muscle fiber typing in routinely processed skeletal muscle with monoclonal antibodies” Histochem. 93:497, 1990; incorporated herein by reference), cytoarchitectural organization consistent with skeletal myocytes, and the presence of normal appearing nuclei located peripherally. Using representative tissue sections and computer-assisted imaging analysis, the areas of engraftment were calculated and converted to the number of engrafted nuclei according to a separate count of nuclei density performed on Trichrome stained sections. The total number of surviving myoblast nuclei in each tissue block was estimated using the following equation:

$$\begin{matrix}Sum of Graft \\ Area in Section\end{matrix} \times \begin{matrix}Density of Nuclei \\ Per Graft Area\end{matrix} \times \begin{matrix}\# Sections \\ Per Block\end{matrix} \times \begin{matrix}Abercrombie \\ {Correction}^{*}\end{matrix}$$

$$( Abercrombie , “ Estimation of nuclear population from microtome sections ” Ant . Rec . 94 \text{:} 239 –47 , 1946 ; incorporated herein by reference ) \\ * Estimated number of sections per block according to aproximate block thickness of 3 mm and section thickness of 5 μm .$$

Statistical Analysis

Data are presented as mean ± standard error of mean (SEM). The differences over time and between groups for LV hemodynamic, geometric, and functional data during the 6-week HF study period were studied using multifactoral (two-way) analysis of variance (ANOVA) with repeated measurements (factors: group and time). For HF control, the differences between baseline and HF time points were established using a one-way ANOVA test with repeated measurements. If the F-ratio exceeded a critical value (alpha <0.05) the post-hoc Student-Newman-Keuls method was used to perform pair-wise comparisons. HF+ASM data at HF week 1 was compared to baseline using a t-test.

Individual regression analysis for PV relationships was computed by analysis software (IOX, EMKA Technologies). The equality of the PV relationships for the HF+ASM and HF controls was studied with multiple-linear regression considering both qualitative (group) and interaction terms, i.e. simultaneously testing the differences in slope and intersect of the regression functions. Linear regression analyses were also performed to study the relationship between indices of LV remodeling and function versus the number of surviving ASM-derived myocytes including HF controls (N=11).

In establishing HF as both an increased ESVI and decreased LVEF, a null hypothesis consisting of two variables, a Bonferroni method for multiple comparisons was used with an appropriate level of confidence: alpha <0.025. P-values were determined and considered in assigning significance and importance of comparisons.

Results

Eleven sheep were studied for six weeks after establishment of HF with ASM injection (HF+ASM, N=5) or without (HF control, N=6). Three of 8 sheep intended for the HF+ASM group died during the instrumentation procedure; either before ASM injection (N=2) or within 72 hours after injection, and were not included in the study. No sheep in the HF controls died early. Animals had maintenance of appetite and weight over the six-week study. Sheep were less active after HF induction and dyspneic upon mild exertion, but no differences in daily observations were appreciated between groups.

Histology

The average number of injected myoblasts was 3.44±0.49×10⁸ cells, ranging from 1.53 to 4.3×10⁸ cells. Myoblast purity, 92±1.4%, and cell viability, 93±1.2%, were assessed at the time of transport and myoblast viability was confirmed to be>90% (using trypan blue exclusion) after shipment (4° C.). ASM-derived skeletal myofibers were found in all injected hearts, but the relative survival (see discussion) of injected myoblasts surviving at week 6 ranged from 140,000 cells (0.05% survival) to 33 million cells (10.7% survival).

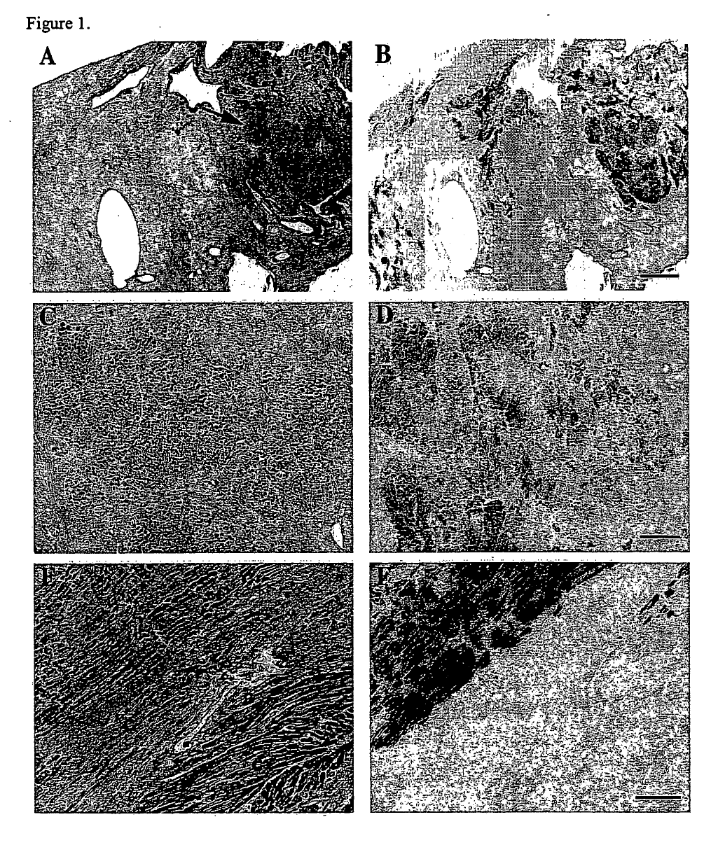

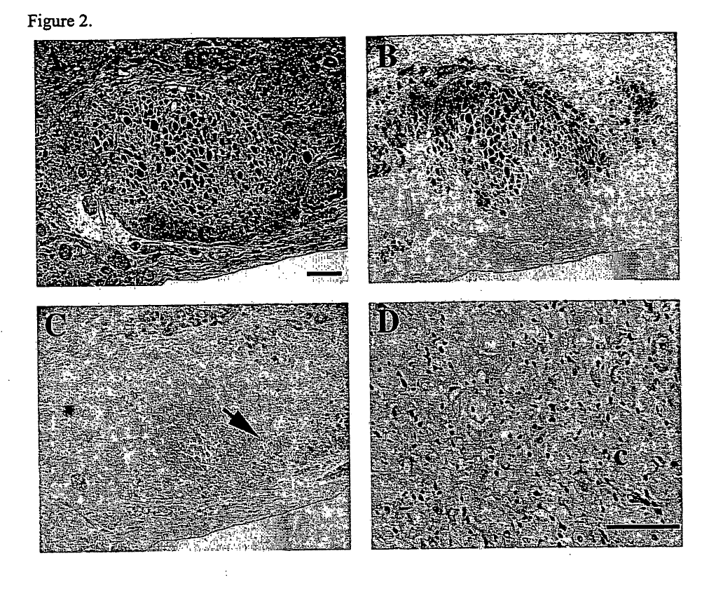

Representative histological sections with detailed descriptions are found in FIGS. 1 and 2. In general, skeletal myocytes were seen aligned with other skeletal muscle fibers as well as aligned with remaining cardiac myocytes (FIG. 1C-F; FIG. 2A, B). Engrafted skeletal muscle fibers were characterized by staining to the myosin heavy chain fast-twitch isoform (purple staining FIGS. 1B, D, F and 2B). However, in no section were ASM-derived myofibers seen stained for troponin I or connexin-43 despite close apposition to surviving cardiac myocytes (FIG. 2 C & D, respectively).

Cardiac Hemodynamics

Hemodynamic data are summarized in Table 1. The study was adequately powered (β≦0.20) to detect a 50% change in LVEF; however, no animal had improvement in dP/dTₘₐₓ or LVEF after ASM injection. No linear relationship was found between the number of surviving cells and LVEF (R²=0.00017, p=0.99) or dP/dTmax (R²=0.048, p=0.543).

TABLE 1
Cardiac Hemodynamics after Autologous Skeletal Myoblast Transplantation

| | | Baseline | HF Ctrl | HF Ctrl | ASM + HF | ASM + HF |
|---|---|---|---|---|---|---|
| | | (N = 6) | Wk 1 (N = 6) | Wk 6 (N = 6) | Wk 1 (N = 5) | Wk 6 (N = 5) |
| Heart Failure | ESVI | 39 ± 4 | 93 ± 7* | 124 ± 15^{‡§} | 85 ± 16* | 98 ± 18^{§} |
| | ml/m² | | | | | |
| | LVEF % | 48 ± 2 | 30 ± 2* | 28 ± 2 | 29 ± 4* | 27 ± 4 |
| HR | | 109 ± 4 | 126 ± 7* | 125 ± 9 | 128 ± 9* | 110 ± 10^{‡} |
| bpm | | | | | | |
| LVSP | | 106 ± 6 | 103 ± 5 | 102 ± 7 | 103 ± 2 | 106 ± 4 |
| mmHg | | | | | | |
| LVEDP | | 11 ± 1 | 23 ± 4* | 26 ± 3 | 21 ± 2* | 22 ± 3 |
| mmHg | | | | | | |
| EDVI | | 75 ± 7 | 135 ± 9* | 170 ± 15^{‡§} | 118 ± 16* | 131 ± 2^{§} |
| ml/m² | | | | | | |
| dP/dTₘₐₓ | | 3414 ± 92 | 2428 ± 327* | 1864 ± 216^{‡} | 2863 ± 152* | 2166 ± 174^{‡} |
| mmHg/sec | | | | | | |
| dP/dTₘᵢₙ | | −2124 ± 108 | −1582 ± 173* | −1423 ± 147 | −1880 ± 68* | −1713 ± 84 |
| mmHg/sec | | | | | | |
| Tau | | 17 ± 1 | 38 ± 8* | 39 ± 4 | 34 ± 4* | 44 ± 3^{‡} |
| ms | | | | | | |
| Pressure Volume | Eₑₛ | 3.7 ± 0.5 | 1.3 ± 0.11* | 0.9 ± 0.13 | 1.6 ± 0.22 | 1.6 ± 0.28 |
| Analysis | Vₒ | 18 ± 3.9 | 39 ± 7.8* | 59 ± 8.7^{‡} | 32 ± 3.1 | 25 ± 3.4^{§} |
| HF + ASM (N = 4), HF | M_{w} | 97 ± 7.4 | 66 ± 10.1* | 58 ± 7.8^{‡} | 67 ± 5.9* | 57 ± 8.0^{‡} |
| ctrl (N = 5) | V_{w} | 50 ± 9.5 | 88 ± 4.9*^{§} | 107 ± 15^{‡§} | 75 ± 3.3*^{§} | 71 ± 2.9^{§} |
| | PVA | 5257 ± 542 | 6943 ± 856 | 8794 ± 449^{‡} | 6456 ± 492 | 6900 ± 497 |
| | PE | 1347 ± 165 | 2982 ± 381* | 4304 ± 375^{‡} | 2791 ± 437* | 3208 ± 500 |
| | SW | 3910 ± 433 | 3961 ± 542 | 4489 ± 710 | 3675 ± 185 | 3688 ± 541 |

Mean ± SEM.
*p < 0.05 from baseline at week 1.
^{‡}p < 0.05 from week 1 within groups.
^{§}p < 0.05 between groups at respective times.
dP/dT: derivative of pressure,
HR: heart rate,
LVSP: LV systolic pressure,
LVEDP: LV end-diastolic pressure,
ESVI and EDVI: LV end-systolic and diastolic volume index,
Tau: time constant of relaxation (Weiss method).
M_{w}: preload recruitable stroke work;
Eₑₛ: end-systolic pressure volume relationship;
Vo: x-intercept of Eₑₛ;
V_{w}: x-ntercept of M_{w};
PE: potential LV energy,
SW: LV stroke work.

Pressure Volume Analysis (PV analysis)

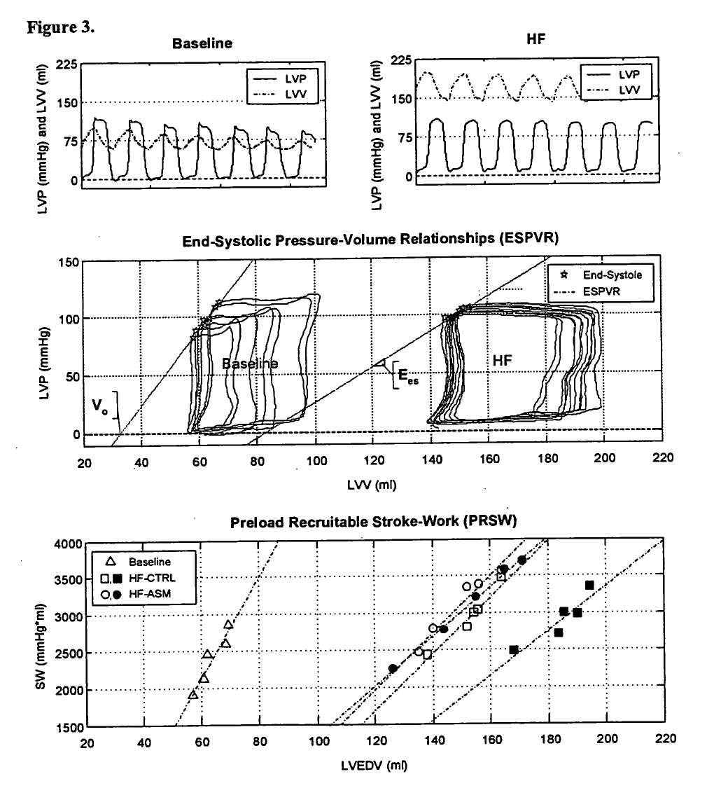

Data for ESPVR, PRSW and LV work (PVA) from HF controls and HF+ASM sheep are summarized in Table 1 and exemplified in FIG. 3. PV analysis demonstrated a decrease in slope of the PRSW (M_{w}) and the load-independent index of cardiac contractility, Eₑₛ, in both groups of HF sheep from baseline. Additional multiple linear regression analyses accounting for covariance between groups also demonstrated no significant differences in slope (WK1: p=0.614, WK6: p=0.519, power=1) or intercept (WK1: p=0.945, WK6: p=0.928, power=1) of the volume-adjusted PV-relationship were found at any time-point. No linear relationship found between the number of surviving cells and Eₑₛ (R²=0.088, p=0.436) or M_{w} (R²=0.018, p=0.731).

There was an increase (rightward shift, p=0.026) in the V₀ (x-intercept) of the Eₑₛ for the HF controls from week 1 to week 6 (FIG. 3). Conversely, for the HF+ASM sheep, the V₀ tended (p=0.20) to decrease (leftward shift) over the six weeks in the HF+ASM animals, and a difference was noted (p=0.014) between HF control and HF+ASM at week six, supporting that ASM injection attenuated LV remodeling. As a result and suggesting a greater loss of myocardial efficiency in HF controls, the PE was increased (p=0.028) in the HF controls from week 1 to week 6, though total LV work (PVA) was not different between groups over the 6-week study. Similar to the V₀, the x-intercept of the PRSW (V_{w}) was increased from week 1 to week 6 in the HF control group (p=0.03), and remained different (p=0.009) as compared to the HF+ASM group at week 6 (Table 1 and FIG. 3).

Sonomicrometry and Left Ventricular Segmental Function

Left ventricular regional and segment data are presented in Table 2. SLₚₒₛₜ was not different in either group from week 1, but was increased (p<0.05 at HF Week 1) from baseline in the HF control group. Left ventricular segmental dyskinesia was present after microembolization, therefore, both systolic bulging (SB) and post-systolic shortening (PSS) were evident in both groups throughout the 6-week study.

TABLE 2
Left Ventricular Regional & Segmental Function after
Autologous Skeletal Myoblast Transplantation

| | Baseline | HF Ctrl | HF Ctrl | HF + ASM | HF + ASM |
|---|---|---|---|---|---|
| | (N = 6) | Wk 1(N = 6) | Wk 6 (N = 6) | Wk 1(N = 5) | Wk 6 (N = 5) |
| SA_{ES} | 37.9 ± 2.5 | 51.8 ± 2.9* | 57.6 ± 3.6^{‡} | 51.5 ± 4.9* | 53.4 ± 4.9 |
| (mm) | | | | | |
| SA | 22.3 ± 1.2 | 14.3 ± 2.3* | 13.3 ± 1.7 | 15.2 ± 0.8* | 13.0 ± 1.4 |
| (% shrt) | | | | | |
| LA_{ES} | 73.2 ± 3.8 | 83.7 ± 5.2* | 90.2 ± 4.6^{‡} | 80.4 ± 4.1 | 85.5 ± 4.9 |
| (mm) | | | | | |
| LA | 11.8 ± 0.9 | 9.8 ± 1.1* | 8.5 ± 1.6 | 8.5 ± 0.9* | 8.1 ± 1.9 |
| (% shrt) | | | | | |
| SL_{post**} | 13.7 ± 3.4 | 17 ± 4.7* | 18.9 ± 5.6 | 12 ± 1.3 | 12.1 ± 1.3 |
| (mm) | | | | | |
| SLₚₒₛₜ | 7.8 ± 1.6 | −1.6 ± 1.1* | −2.1 ± 1.8 | −1.8 ± 2.2* | −2.4 ± 1.8 |
| (% shrt) | | | | | |
| SB | 0.04 ± 0.03 | 0.42 ± 0.10* | 0.53 ± 0.16 | 0.40 ± 0.14* | 0.43 ± 0.18 |
| (mm) | | | | | |
| PSS | 0.06 ± 0.04 | 0.47 ± 0.13* | 0.61 ± 0.2 | 0.45 ± 0.13* | 0.57 ± 0.12 |
| (mm) | | | | | |

Mean ± SEM.
*p < 0.05 from baseline.
^{‡}p < 0.05 from Wk 1 within groups.
^{§}p < 0.05 between HF control and HF + ASM.
ES: end-systolic,
SA: LV short-axis,
LA: LV long-axis,
SLₚₒₛₜ: posterior LV segment (microembolized),
% shrt: % systolic shortening,
SB: systolic bulging,
PSS: post-systolic shortening.
**note: SLₚₒₛₜ length differs from baseline to HF ctrl due to regional infarct expansion after instrumentation, whereas HF + ASM does not differ from baseline because instrumentation of HF + ASM animals was after heart failure (after infarct expansion). Therefore, the relevant comparison of groups is from week 1 to week 6.

Sonomicrometry and Left Ventricular Dimensions

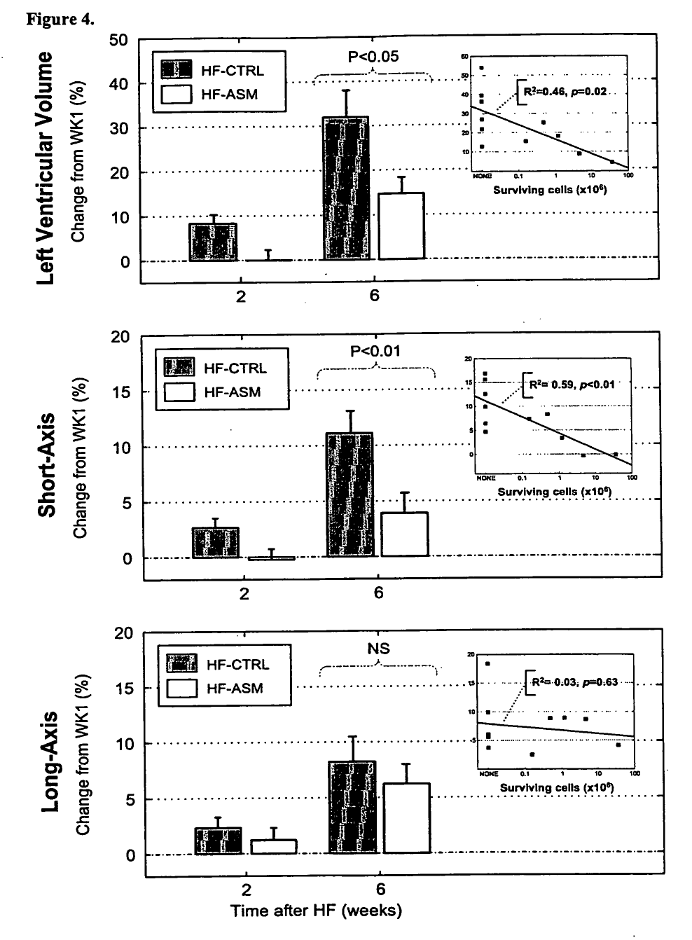

Left ventricular end-systolic and end-diastolic volume indexes (ESVI and EDVI, respectively) were increased (p<0.05) from baseline in both groups at HF week 1, however, there was no difference between groups at week 1 (Table 1). In HF+ASM, LV dilatation was attenuated as compared to HF controls (p=0.0 16) by week 3 (% change in ESVI: 5.3±1.2 % and 17.8±3.3%, respectively) and progressed (p=0.006) by week 6 (FIG. 4). The difference in LV volume resulted from a significant (p=0.005) attenuation in SA dilatation alone and also by week 3 in HF+ASM (FIG. 4). No difference (P>0.5) was found in LA dilatation between groups. Correlations of ESVI, SA and LA to ASM survival are presented in FIG. 4.

Discussion

Previous studies have suggested that skeletal myoblasts form viable skeletal muscle grafts that presumably contributed to improved cardiac performance and remodeling after experimental myocardial infarction. Few studies, if any, have examined the impact of ASM in hearts with a pre-existing and clinically significant and severe degree of ischemic dysfunction and remodeling (LVEF<35% with LVESVI>80 ml/m²). The present study estalishes the therapeutic benefit of ASM cardiomyoplasty in a clinically applicable model of ischemic, dilated heart failure free of the confounding factors associated with coronary revascularization or other supportive therapies.

ASM-derived skeletal muscle was found in all injected sheep at six weeks. We report here an estimate of survival that allowed the relative survival between animals to be compared. Because significant limitations exist in the method used to calculate cell survival (Abercrombie, “Estimation of nuclear population from microtome sections” Ant. Rec. 94:239-47, 1946; incorporated herein by reference), values for cell survival should not be interpreted as absolute cell survival. The long-term survival of myoblasts (up to 10.7% survival) found in this study was higher than reported in patients transplanted with a similar number of ASM cells at the time of LVAD placement (<1% survival) (Pagani et al. “Autologous skeletal myoblasts transplanted to ischemia-damaged myocardium in humans” J. Am. Coll. Cardiol. 41:879-888, 2003; incorporated herein by reference). As others have reported (Scorsin et al. “Comparison of effects of fetal cadiomyocyte and skeletal myoblast transplantation on postinfarction left ventricular function” J. Thor. Cardiovasc. Surg. 2000; 119:1169-75; Jain et al. “Cell therapy attenuated deleterious ventricular remodeling and improves cardiac performance after myocardial infarction” Circulation 103:1920-1927, 2000; Ghostine et al. “Long-term efficacy of myoblast transplantation on regional structure and function after myocardial infarction” Circulation 106[suppl I]:I131-6, 2002; Menasche et al. “Myoblast transplantation for heart failure” Lancet 357: 279-80, 2001; Menasche et al. “Autologous skeletal myoblast transplantation for severe postinfarction left ventricular dysfunction” J. Am. Coll. Cardiol. 41:1078-83, 2003; Pagani et al. “Autologous skeletal myoblasts transplanted to ischemia-damaged myocardium in humans” J. Am. Coll. Cardiol. 41:879-888, 2003; Pouzet et al. “Factors affecting functional outcome after autologous skeletal myoblast transplantation” Ann. Thorac. Surg. 71:844-851, 2001; each of which is incorporated herein by reference), no staining for connexin-43 was found in ASM-derived skeletal muscle. Transplanted ASM-derived skeletal myofibers aligned with each other and with remaining cardiac myofibers in all sections (FIGS. 1 and 2). Such organized alignment of the ASM-derived fibers suggests that these fibers remained sensitive to stress-strain relationships found within the myocardium (Kada et al. “Orientation change of cardiocytes induced by cyclic stretch stimulation: time dependency and involvement of protein kinases” J. Mol. Cell. Cardiol. 31:247-59, 1999; Pfeffer et al. “Ventricular remodeling after myocardial infarction. Experimental observations and clinical implications” Circulation 81:1161-72, 1990; Atkins et al. “Intracardiac transplantation of skeletal myoblasts yields two populations of striated cells in situ” Ann. Thorac. Surg. 67:124-9, 1999; each of which is incorporated herein by reference). Meaningful estimation of scar replacement after ASM injection was not possible given the heterogeneous infarct pattern present after microembolization and the relatively few animals studied. However, we consistently observed cells surviving aligned with each other in dense myocardial infarct (FIG. 1) and less frequently found cells in close proximity to surviving cardiac myocytes (FIG. 2).

Even with relatively low myoblast cell survival (FIG. 1, animal with 1.1% cell survival), considerable areas of scarred myocardium can be filled with viable myofibers as a result of cell fusion and subsequent enlargement of myofibers (approximately 10-fold increase in myofiber cross-sectional area per nucleus versus myoblasts, unpublished observations). Thus, it may be possible to completely fill damaged areas in the myocardium even with low cellular survival. In general, up to 95% of the injected cells are lost shortly after injection (Menasche, “Myoblast-based cell transplantation” Heart Failure Reviews. 8:221-27, 2003; Grossman et al. “Incomplete Retention after Direct Myocardial Injection” Catheterization and Cardiovascular Interventions 55:392-397, 2002; each of which is incorporated herein by reference). An explanation for this early loss is by means of lymphatic and/or venous drainage of the cells after direct intramyocardial injection (Grossman et al. “Incomplete Retention after Direct Myocardial Injection” Catheterization and Cardiovascular Interventions 55:392-397, 2002; incorporated herein by reference). Other factors also likely contribute to the further loss of cells that are retained within the myocardium/scar. Recent, investigations have shown that both the pre-treatment (Retuerto et al. “Angiogenic pre-treatment improves the efficacy of cellular cardiomyoplasty performed with fetal cardiomyocyte implantation” J. Thorac. Cardiovasc. Surg. 127: 1-11, 2004; incorporated herein by reference) and transfection (Askari et al. “Cellular, but not direct, adenoviral delivery of vascular endothelial growth factor results in the improved left ventricular function and neovascularization in dilated ischemic cardiomyopathy” JACC 43:1908-14, 2004; incorporated herein by reference) of ASM with VEGF improved cardiac function, presumably by enhancing perfusion and nutrient delivery. Furthermore, strategies to both limit inflammation and/or apoptosis have also proven beneficial to improving the efficacy after cellular cardiomyoplasty (Zhang et al. “Cardiomyocyte grafting for cardiac repair: graft cell death and anti-death strategies” J. Mol. Cell. Cardiol. 33:907-21, 2001; each of which is incorporated herein by reference). No evidence for intense inflammation at the graft sites 6 weeks after injection was observed (FIGS. 1 and 2).

Left Ventricular Function

Data evaluating cardiac performance after ASM injection in Tables 1 and 2-suggests no improvement in any hemodynamic parameter or in index of cardiac contractility in sheep with end-stage, dilated ischemic HF in this model. This discrepancy with results previously published in sheep (Ghostine et al. “Long-term efficacy of myoblast transplantation on regional structure and function after myocardial infarction” Circulation 106[suppl I]:I131-6, 2002; incorporated herein by reference) may be due to the worse LV structure and function in our sheep. Ghostine and colleagues improvement in local cardiac function may have been as a result of less severe pathology, and therefore, less of an impediment to ASM contraction if present.

Unlike, Pouzet and colleagues (Pouzet et al. “Factors affecting functional outcome after autologous skeletal myoblast transplantation” Ann. Thorac. Surg. 71:844-851, 2001; incorporated herein by reference) who demonstrated in rats stratified for LV function (LVEF) a significant correlation with the number of cells injected to indices of LV function; those most severely impaired received the greatest benefit, we were unable to demonstrate such a relationship compared to the number of surviving ASM-derived myocytes. Beyond the obvious difference in comparing the number of injected cells versus that of the percentage surviving, could this difference be explained by a difference in myoblast culture, expansion or possibly just an insufficient dose of cells? Pouzet et al. and Ghostine et al. present myoblast purity less than 50% at time of injection, whereas we expanded a more pure population of myoblasts (>90% CD56 positive). Could the purity of myoblast injection suspensions impact outcome? Although this is possible, it would seem unlikely that higher myoblast purity would result in diminished functional benefits; moreover, understanding the impact of cell culture and expansion techniques is difficult given the variability in LV pathology in ours and other published animal studies (Ghostine et al. “Long-term efficacy of myoblast transplantation on regional structure and function after myocardial infarction” Circulation 106[suppl I]:I131-6, 2002; Menasche et al. “Myoblast transplantation for heart failure” Lancet 357:279-80, 2001; Menasche et al. “Autologous skeletal myoblast transplantation for severe postinfarction left ventricular dysfunction” J. Am. Coll. Cardiol. 41:1078-83, 2003; Pagani et al. “Autologous skeletal myoblasts transplanted to ischemia-damaged myocardium in humans” J. Am. Coll. Cardiol. 41:879-888, 2003; Pouzet et al. “Factors affecting functional outcome after autologous skeletal myoblast transplantation” Ann. Thorac. Surg. 71:844-851, 2001; each of which is incorporated herein by reference).

The lack of a demonstrable direct functional benefit observed in our study may be related to the chronic nature and severity of LV dysfunction in our HF model (multiple microinfarctions over several weeks), as compared to animal models using a single ischemic insult (cryoinfarction (Taylor et al. “Regenerating functional myocardium: improved performance after skeletal myoblast transplantation” Nat. Med. 4(8):929-33, 1998; incorporated herein by reference), ligation (Jain et al. “Cell therapy attenuated deleterious ventricular remodeling and improves cardiac performance after myocardial infarction” Circulation 103:1920-1927, 2000; incorporated herein by reference), coil embolization (Ghostine et al. “Long-term efficacy of myoblast transplantation on regional structure and function after myocardial infarction” Circulation 106[suppl I]:I131-6, 2002; incorporated herein by reference)). The microembolization model may have more effectively exhausted remote myocardial compensatory mechanisms, by design (Sabbah et al. “A canine model of chronic heart failure produced by multiple sequential coronary microembolizations” Am. J. Physiol. 260:H1379-84, 1991; incorporated herein by reference), preventing contribution from the remote myocardium after ASM injection. We agree with the interpretation offered by Jain et al. (“Cell therapy attenuated deleterious ventricular remodeling and improves cardiac performance after myocardial infarction” Circulation 103:1920-1927, 2000; incorporated herein by reference), in their ex vivo preparation in rats, that modest functional improvements observed after ASM injection were likely the result of benefits to non-functional properties of the LV, i.e., attenuated LV dilatation, rather than directly to LV contraction. In essence, less wall stress placed on remote cardiac myocytes as a result of ASM-derived skeletal muscle preventing further LV chamber dilatation would translate into better remote myocardial function. Perhaps the earlier the treatment the sooner the benefits of ASM-derived skeletal muscle could be realized on LV remodeling, and therefore greater the likelihood that the remote cardiomyocytes could adequately compensate and contribute to global LV function? Likewise, we believe based on our studies that with more severe dilation longer periods may be required for functional changes to be observed.

Left Ventricular Remodeling

An important finding of the present study was the attenuation of LV dilatation after ASM transplantation in a cell survival dependent fashion (FIG. 4). Studies in both large and smaller animals have also shown positive effects on LV dilatation after ASM injection (Tambara et al. “Transplanted skeletal myoblasts can fully replace the infarcted myocardium when they survive in the host in large numbers” Circulation 108[suppl II]:II-259-63, 2003; Taylor et al. “Regenerating functional myocardium: improved performance after skeletal myoblast transplantation” Nat. Med. 4(8):929-33, 1998; Jain et al. “Cell therapy attenuated deleterious ventricular remodeling and improves cardiac performance after myocardial infarction” Circulation 103:1920-1927, 2000; Ghostine et al. “Long-term efficacy of myoblast transplantation on regional structure and function after myocardial infarction” Circulation 106[suppl I]:I131-6, 2002; Pouzet et al. “Factors affecting functional outcome after autologous skeletal myoblast transplantation” Ann. Thorac. Surg. 71:844-851, 2001; incorporated herein by reference). Another intriguing finding of the current study was that effects on LV dilatation were exclusively for the SA dimension. The mechanism(s) that defines this preferential effect on SA remodeling is not entirely clear. The idea that cellular cardiomyoplasty may be directly impacting scar elasticity and thereby limiting scar expansion is a possible explanation for attenuated regional dilatation (Torrent-Guasp et al. “The structure and function of the helical heart and its buttress wrapping. Articles I-VII” Semin. Thor. and Cardiovasc. Surg. 13: 298-416, 2001; incorporated herein by reference). Though the interplay of both post systolic shortening and systolic bulging in chronically ischemic myocardium has not been well characterized (Skulstad et al. “Postsystolic shortening in ischemic myocardium, active contraction or passive recoil” Circulation 106:718-24, 2002; incorporated herein by reference), the fact remains that there were no measurable improvements after ASM injection in either PSS or SB.

If ASM-derived skeletal myofibers can actively resist forces (stretch) inline with their fibers, as demonstrated ex vivo (Murry et al. “Skeletal myoblast transplantation for repair of myocardial necrosis” J. Clin. Invest. 98:2512-23, 1996; incorporated herein by reference), and thereby limit LV dilatation, this might also explain the observed attenuation to LV dilatation selectively for the LV short axis. For example: as the ventricle becomes increasingly spherical after ischemic injury, the predominate cardiac fiber axis (e.g., 60°) progressively re-orients towards the horizontal or short-axis (e.g., 30°) (Torrent-Guasp et al. “The structure and function of the helical heart and its buttress wrapping. Articles I-VII” Semin. Thor. and Cardiovasc. Surg. 13: 298-416, 2001; incorporated herein by reference). We provide evidence that ASM-derived skeletal myofibers were found aligned with each other and with remaining cardiac myocytes and therefore, theoretically, the engrafted ASM-derived myofibers' orientation would be more aligned with the LV short axis. As suggested, ASM-derived myofibers may offer innate resistance to dilatory forces upon or along their fiber lengths, thereby, selectively preventing dilatation aligned with ASM engraftment along the LV short axis (FIG. 4).

Like Jain et al. (“Cell therapy attenuated deleterious ventricular remodeling and improves cardiac performance after myocardial infarction” Circulation 103:1920-1927, 2000; incorporated herein by reference), in an isolated heart preparation, we found that ASM cardiomyoplasty prevented a rightward shift of the Eₑₛ intercept (V₀) as well as the intercept for the PRSW (Table 1 and FIG. 4). In light of and in an attempt to meaningfully quantify the apparent discordant effects of ASM transplantation on LV remodeling versus that of LV function, we calculated PVA from acquired pressure volume data (Todaka et al. “Characterizing ventricular mechanics and energetics following repeated coronary microembolizations” Am. J. Physiol. 272:H 186-94, 1997; Recchia et al. “Reduced nitric oxide production and altered myocardial metabolism during the decompensation of pacing induced heart failure in the conscious dog” Cir. Res. 83(10):969-79, 1998; Takaoka et al. “Depressed contractile state and increased myocardial consumption for non-mechanical work in patients with heart failure due to old myocardial infarction” Cardiovasc. Res. 28:1251-7, 1994; each of which is incorporated herein by reference). The fact that the increase in the non-mechanical cardiac work or PE (Table 1) was attenuated after ASM transplantation suggests a benefit to the mechanoenergetics of the heart. Such a benefit may allow for better cardiac performance overtime and this is supported by the fact that ASM animals had no further deterioration in their Ees over the six weeks and that given more time this may have proven to be significant between groups. Studies are currently underway to evaluate whether improvements in cardiac function may be demonstrated at longer time points or after transplantation of a greater number of ASM cells.

Study Limitations

The animal model used in the present study approximates clinical ischemic HF in etiology, degree of pathology and coronary anatomy (Sabbah et al. “A canine model of chronic heart failure produced by multiple sequential coronary microembolizations” Am. J. Physiol. 260:H1379-84, 1991; Pfeffer et al. “Ventricular remodeling after myocardial infarction. Experimental observations and clinical implications” Circulation 81:1161-72, 1990; Menasche, “Myoblast-based cell transplantation” Heart Failure Reviews 8:221-27, 2003; each of which is incorporated herein by reference). Microembolization does not fully model the phenomenon of myocardial infarction leading to ischemic HF in all patients, particularly those patients who suffer a single large infarct. Moreover, this model greatly accelerates the disease progression typical for chronic ischemic HF (Pfeffer et al. “Ventricular remodeling after myocardial infarction. Experimental observations and clinical implications” Circulation 81: 1161-72, 1990; Pfeffer, “Left ventricular remodeling after acute myocardial infarction” Annu. Rev. Med. 46:455-66, 1995; each of which is incorporated herein by reference).

Each animal underwent the same number and types of procedures as well as being subjected to the same hemodynamic criteria for determination of HF. Differences found in the present study could have resulted based on the timing of instrumentation (and ASM injection) between the groups. The fact that attenuated dilatation was observed and correlated only in the SA dimension in HF+ASM animals, while LA dilatation was nearly identical between the HF control and ASM groups, further support that differences seen between groups were less dependent upon procedural order than on myoblast injection.

Segmental and/or regional function as measured by sonomicrometry may have not adequately documented function in the exact area of ASM engraftment due to the variability of ASM survival; however, myoblast injection was specifically targeted to and was found in the immediate vicinity of the sonomicrometry crystals at 6 weeks. If regional instrumentation failed to reveal functional benefit after ASM injection, then indices such as ESPVR (Eₑₛ) and PRSW (M_{w}) should have remained sensitive to changes in LV volume in relation to chamber pressures to account for the impact of ASM injection. As previously recognized, the method described by Abercrombie (“Estimation of nuclear population from microtome sections” Ant. Rec. 94:239-47, 1946; incorporated herein by reference) is a standardized approach to quantify cell numbers. It is a best estimate for the number of cells surviving, but sampling error is its major limitation. Lastly, the lack of observed benefit to LV function after ASM injection may be related to the limited period of study (Ghostine et al. “Long-term efficacy of myoblast transplantation on regional structure and function after myocardial infarction” Circulation 106[suppl I]:I131-6, 2002; incorporated herein by reference).

Conclusions

The present study describes ASM transplantation in a clinically applicable large animal model of chronic ischemic HF free of concomitant interventions. Despite the apparent lack of direct functional impact on cardiac function, we were able to demonstrate a significant attenuation in LV dilatation after ASM transplantation. The attenuation in LV dilatation was exclusive to the short axis and was observed in a cell survival-dependent fashion. These observations suggest that ASM impact LV remodeling by a mechanism independent of cell-to-cell communication and/or direct functional improvements, but that ASM engraftment and alignment may play a role in such a mechanism.

### Example 2

### Treating Chronic Heart Failure using Inventive Pro-angiogenic Cell Implantation Strategies

Background and Significance

Fifty percent of deaths attributed to cardiovascular disease result from coronary artery disease (CAD), a condition associated with narrowing of the coronary arteries, and reduced blood flow to the heart. Although there has been a 54% decrease in mortality from CAD since 1967 due to continued advances in the treatment of cardiovascular diseases by medical and surgical therapies and preventative measures, CAD remains the leading killer of men and women in the United States (American Heart Association. Heart and Stroke Statistical Update, 2003; incorporated herein by reference). Aside from the burden of morbidity and mortality to these individuals, the societal economic burden of CAD is significant, with an estimated annual cost of $56 billion (Goldfarb et al. “Impact of appropriate pharmaceutical therapy for chronic conditions on direct medical costs and workplace productivity: a review of the literature” Dis. Manag. 7(1):61-75, 2004; incorporated herein by reference). A large proportion of these dollars are spent treating patients suffering from heart failure. The total direct costs of heart failure are predicted to exceed $22.1 billion in 2003. While total costs include the costs of hospitalizations, physicians' office visits, nursing home stays, home health care, and pharmacotherapy, the main cost drivers are frequent hospitalizations and readmissions. Readmission costs account for almost 30% of total inpatient care costs (Goldfarb et al. “Impact of appropriate pharmaceutical therapy for chronic conditions on direct medical costs and workplace productivity: a review of the literature” Dis. Manag. 2004;7(1):61-75). Effective strategies must be developed which can regenerate myocardium in patients suffering from chronic ischemic heart failure.

Current Therapy

Pharmacologic therapy is a mainstay of treatment for most forms of CAD, and is limited in its ability to reverse coronary atherosclerotic lesions. Mechanical revascularization by percutaneous transluminal coronary angioplasty (PTCA) results in the reversal of ischemia and frequently improved global and regional left ventricular function (Zijlstra et al. “A comparison of immediate coronary angioplasty with intravenous streptokinase in acute myocardial infarction” Circulation 89: 68-75, 1994; Bolognese et al. “Left ventricular remodeling after primary coronary angioplasty: patterns of left ventricular dilation and long-term prognostic implications” Circulation 106:2351-57, 2002; each of which is incorporated herein by reference). Coronary artery bypass graft surgery (CABG), is a procedure whereby venous or arterial conduit is used to bypass the coronary occlusion. Similar five year survival rates are associated with medical (81%) and surgical (84%) treatment of CAD (American Heart Association. Heart and Stroke Statistical Update, 2003; incorporated herein by reference). The application of these therapies to patients with myocardial scar, severe left ventricular dysfunction, and left ventricular dilatation remains controversial and of marginal benefit (Bolling et al. “Intermediate-term outcome of mitral reconstruction in cardiomyopathy” J. Thor. Cardiovasc. Surg. 115:381-88, 1998; Trachiotis et al. “Coronary artery bypass grafting in patients with advanced left ventricular dysfunction” Ann. Thor. Surg. 66:1632-39, 1998; Cope et al. “A cost comparison of heart transplantation versus alternative operations for cardiomyopathy” Ann. Thor. Surg 72:1298-305, 2001; each of which is incorporated herein by reference). It is estimated that approximately 100,000 patients/year with ischemic heart disease may not be candidates for conventional therapies. More complex procedures such as heart transplantation, artificial heart devices, ventricular remodeling procedures are applicable to select patient populations, but they do not treat the underlying myocardial disease and are further limited by cost, availability, efficacy, and/or morbidity (O'Connell et al. “Economic impact of heart failure in the United States: time for a different approach” J. Heart Lung Transplant. 13:S107-S112, 1994; Rose et al. “Long-term use of a left ventricular assist device for end-stage heart failure” NEJM 345(20):1435-43, 2001; Taylor et al. “The registry of the international society of heart and lung transplantation: 20^{th} official adult heart transplant report-2003” J. Heart Lung Transplant. 22(6):616-624, 2003; Cope et al. “A cost comparison of heart transplantation versus alternative operations for cardiomyopathy. Ann. Thor. Surg. 72:1298-305, 2001; each of which is incorporated herein by reference).

Chronic Ischemia, Remodeling, and Ischemic Heart Failure (CHF)

Cardiac myocytes are quickly and often irreversibly damaged by even relatively short periods of ischemia (Sutton et al. “Left ventricular remodeling after myocardial infarction: pathophysiology and therapy” Circulation 101:2981-88, 2000; incorporated herein by reference). The high metabolic demands of cardiac tissues make them particularly susceptible to ischemia and reperfusion injury. Since cardiac myocytes lack an effective self-regenerative capacity, fibrous connective tissue and scar replace dead cardiac myocytes after myocardial infarction (Sutton et al. “Left ventricular remodeling after myocardial infarction: pathophysiology and therapy” Circulation 101:2981-88, 2000; Pfeffer et al. “Ventricular Remodeling after myocardial infarction: experimental observations and clinical implications” Circulation 81:1161-72, 1990; Bodi et al. “Wall motion of non-infarcted myocardium: relationship to regional and global systolic function and to early and late left ventricular dilatation” Inter. J. Card. 71:157-65, 1999; each of which is incorporated herein by reference). No current intervention can restore infarcted heart muscle to its original functional capacity (Bolognese et al. “Left ventricular remodeling after primary coronary angioplasty: patterns of left ventricular dilation and long-term prognostic implications” Circulation 106:2351-57, 2002; incorporated herein by reference). Regional loss of contractility results in reduced left ventricular ejection fraction (LVEF) that over time can lead to enlargement of the heart as it attempts to maintain cardiac output by maintaining stroke volume (Pfeffer et al. “Ventricular remodeling after myocardial infarction: experimental observations and clinical implications” Circulation 81:1161-72, 1990; Pfeffer, “Left ventricular remodeling after acute myocardial infarction” Annu. Rev. Med. 46:455-66, 1995; each of which is incorporated herein by reference).

Ventricular enlargement (remodeling) results in an increase in myocardial wall stress, thus further limiting remote myocyte function, as simply stated by Laplace's Law (Mitchell et al. “Left ventricular remodeling in the year after first anterior myocardial infarction: a quantitative analysis of contractile segment lengths and ventricular shape” J. Am. Coll. Cardiol. 19:1136-44, 1992; Bodi et al. “Wall motion of non-infarcted myocardium: relationship to regional and global systolic function and to early and late left ventricular dilatation” Inter. J. Card. 71:157-65, 1999; Pfeffer, “Left ventricular remodeling after acute myocardial infarction” Annu. Rev. Med. 46:455-66, 1995; each of which is incorporated herein by reference). Moreover, mechanisms that result in increasing ventricular pressures and/or increasing chamber volumes and/or increasing wall thinning would result in further myocardial wall stress, driving this paradigm of the failing heart (Pfeffer et al. “Ventricular remodeling after myocardial infarction: experimental observations and clinical implications” Circulation 81:1161-72, 1990; Pfeffer, “Left ventricular remodeling after acute myocardial infarction” Annu. Rev. Med. 46:455-66, 1995; Cohn, “Structural basis for heart failure, ventricular remodeling and its pharmacologic inhibition” Circulation 91:2504-07, 1995; each of which is incorporated herein by reference).

White et al (“Left ventricular end-systolic volume as the major determinant of survival after recovery from myocardial infarction” Circulation 76(1):44-51, 1987; incorporated herein by reference) and the GUSTO I trial (Migrino et al. “End-systolic volume index at 90 and 180 minutes into reperfusion therapy for acute myocardial infarction is a strong predictor of early and late mortality” Circulation 96:116-121, 1997; incorporated herein by reference) have documented that left ventricular dilatation following myocardial infarction is an independent and significant predictor of mortality. Therefore, early survival after myocardial infarction may be predicated by the timeliness and adequacy of appropriate reperfusion therapy, but long-term prognosis is strongly dependent on subsequent changes in left ventricular geometry and function (Mitchell et al. “Left ventricular remodeling in the year after first anterior myocardial infarction: a quantitative analysis of contractile segment lengths and ventricular shape” J. Am. Coll. Cardiol. 19:1136-44, 1992; White et al. “Left ventricular end-systolic volume as the major determinant of survival after recovery from myocardial infarction” Circulation 76(1):44-51, 1987; Gheorghiade et al. “Chronic heart failure in the United States, a manifestation of coronary artery disease” Circulation 97:282-89, 1998; Perin et al. “Transendocardial, autologous bone marrow cell transplantation for severe, chronic ischemic heart failure” Circulation 107:2294-2302, 2003; each of which is incorporated herein by reference) leading to congestive heart failure (CHF) and eventually death (Mitchell et al. “Left ventricular remodeling in the year after first anterior myocardial infarction: a quantitative analysis of contractile segment lengths and ventricular shape” J. Am. Coll. Cardiol. 19:1136-44, 1992; Migrino et al. “End-systolic volume index at 90 and 180 minutes into reperfusion therapy for acute myocardial infarction is a strong predictor of early and late mortality” Circulation 96:116-121, 1997; White et al. “Left ventricular end-systolic volume as the major determinant of survival after recovery from myocardial infarction” Circulation 76(1):44-51, 1987; Pfeffer, “Left ventricular remodeling after acute myocardial infarction” Annu. Rev. Med. 46:455-66, 1995; Cohn, “Structural basis for heart failure, ventricular remodeling and its pharmacologic inhibition” Circulation 91:2504-07, 1995; each of which is incorporated herein by reference).

Cell-based Therapies for Ischemic Heart Failure

Emerging evidence suggests that cardiac myocyte replication and /or cell fusion may occur (Muller et al. “Cardiomyocytes of non-cardiac origin in myocardial biopsies of human transplanted hearts” Circulation 106:31-35, 2002; Beltrami et al. “Evidence that human cardiac myocytes divide after myocardial infarction” N. Engl. J. Med. 44:1750-57, 2001; each of which is incorporated herein by reference), and that resident cardiac “reserve” cells may “auto-regenerate” injured myocardium. The capacity to auto-regenerate myocardium is clinically ineffective and, at least operationally, it is generally believed that adult cardiac muscle cannot regenerate itself after myocyte death. Cell-based therapies or cellular cardiomyoplasty refers to the technique of administering immature cells to the diseased heart, such cells as skeletal myoblasts (satellite cells), bone marrow-derived mesenchymal stem cells, embryonic stem cells, fetal cardiomyocyte, any of which may integrate structurally and functionally into infarcted myocardium (Chiu et al. “Cellular cardiomyoplasty: myocardial regeneration with satellite cell implantation” Ann. Thor. Surg. 60:12-18, 1995; Pagani et al. “Autologous skeletal myoblasts transplanted to ischemia-damaged myocardium in humans” J. Am. Coll. Cardiol. 41:879-888, 2003; Ghostine et al. “Long-term efficacy of myoblast transplantation on regional structure and function after myocardial infarction” Circulation 106[suppl I]:I131-6, 2002; Dorfman et al. “Myocardial tissue engineering with autologous myoblast implantation” J. Thor. Cardiovasc. Surg. 116:744-51, 1998; Taylor et al. “Regenerating functional myocardium: improved performance after skeletal myoblast transplantation” Nat. Med. 4(8):929-33, 1998; Retuerto et al. “Angiogenic pre-treatment improves the efficacy of cellular cardiomyoplasty performed with fetal cardiomyocyte implantation” J. Thorac. Cardiovasc. Surg. 127: 1-11, 2004; Jain et al. “Cell therapy attenuates deleterious ventricular remodeling and improves cardiac performance after myocardial infarction” Circulation 103:1920-27, 2001; Reinecke et al. “Evidence for fusion between cardiac and skeletal muscle cells” Circ. Res. 94(6):e56-60, 2004; McConnell et. al. “Correlation of autologous skeletal myoblast survival with changes in left ventricular remodeling in dilated ischemic heart failure” J. Thorac. Cardiovasc. Surg. 2004 (in press); Kessler et al. “Myoblast cell grafting into heart muscle: cellular biology and potential applications” Annu. Rev. Physiol. 61:219-42, 1999; Yoo et al. “Heart cell transplantation improves heart function in dilated cardiomyopathic hamsters” Circulation 102(19 Suppl 3):III204-9, 2000; Koh et al. “Stable fetal cardiomyocyte grafts in the hearts of dystrophic mice and dogs” J. Clin. Invest. 96(4):2034-42, 1995; Klug et al. “Genetically selected cardiomyocytes from differentiating embronic stem cells form stable intracardiac grafts” J. Clin. Invest. 98(1):216-24, 1996; Jackson et al. “Regeneration of ischemic cardiac muscle and vascular endothelium by adult stem cells” J. Clin. Invest. 107(11):1395-402, 2001; Kocher et al. “Neovascularization of ischemic myocardium by human bone marrow derived angioblasts prevents cardiomyocyte apoptosis, reduces remodeling and improves cardiac function” Nature Medicine 7:430-436, 2001; Kamihata et al. “Implantation of bone marrow mononuclear cells into ischemic myocardium enhances collateral perfusion and regional function via side supply of angioblasts, angiogenic ligands, and cytokines” Circulation 104:1046-1052, 2001; Orlic et al. “Transplanted adult bone marrow cells repair myocardial infarcts in mice” Ann. N.Y Acad. Sci. 938:221-29, discussion 229-30, 2001; Orlic et al. “Mobilized bone marrow cells repair the infarcted heart, improving function and survival” Proc. Natl. Acad. Sci. USA 98:10344-9, 2001; Balsam et al. “Haematopoietic stem cells adopt mature haematopoietic fates in ischaemic myocardium” Nature 428(6983):668-73, 2004; Reinecke et al. “Taking the toll after cardiomyocyte grafting: a reminder of the importance of quantitative biology” J. Mol. Cell. Cardiology 34:251-253, 2002; Perin et al. “Transendocardial, autologous bone marrow cell transplantation for severe, chronic ischemic heart failure” Circulation 107:2294-2302, 2003; each of which is incorporated herein by reference). The implanted cells, usually harvested autogenously and expanded in culture, “repopulate” the area of myocardial scar, presumably with viable myocytes and/or blood vessels. Cellular cardiomyoplasty has been demonstrated in numerous studies to typically result in 10-30% improvements in ventricular function.

One limitation of cell transplantation is the failure of a great majority of cells to survive implantation (see Table 3 below) due to several mechanisms, including the effects of local ischemia, mechanical stress, and early (1-5 hours after implantation) cell loss through lymphatics and venules (Grossman et al. “Incomplete Retention after Direct Myocardial Injection” Catheterization and Cardiovascular Interventions 55:392-397, 2002; incorporated herein by reference)

TABLE 3
Retention of Myoblasts in Tissues

| | Cell Number | Percent |
|---|---|---|
| Tissue sample | in Tissue | of Cells* |
| Brain, Kidney, liver | 0 | 0 |
| Lung | 5,616,687 | 5.1% |
| Anterior LV wall 1 | 2,068,321 | 1.9% |
| Anterior LV wall 2 | 3,333 | <0.1% |
| Lateral LV wall 1 | 49,800 | <0.1% |
| Lateral LV wall 2 | 2,065 | <0.1% |
| Inferior LV wall 1 | 4,093 | <0.1% |
| Inferior LV wall 2 | 4,605 | <0.1% |
| Septal wall 1 | 95,414 | <0.1% |
| Septal wall 2 | 2,286,909 | 2.1% |
| Total Apical Heart | 4,514,539 | 4.1% |

*Percent of 110,600,000 cells injected which were retained in the tissue sample

Because only sparse implanted cell deposition has been found in a number of cell transfer studies, usually <1% graft survival, it has alternatively been postulated that the improvements conferred by cell implantation are related to the angiogenic potential of implanted cells (Kocher et al. “Neovascularization of ischemic myocardium by human bone marrow derived angioblasts prevents cardiomyocyte apoptosis, reduces remodeling and improves cardiac function” Nature Medicine 7:430-436, 2001; Kamihata et al. “Implantation of bone marrow mononuclear cells into ischemic myocardium enhances collateral perfusion and regional function via side supply of angioblasts, angiogenic ligands, and cytokines” Circulation 104:1046-1052, 2001; each of which is incorporated herein by reference). Several investigators have transfected cells with angiogenic mediators or simultaneously after implantation administered both, but the time line of vessel development and increase in perfusion with these strategies as opposed to cell death suggest that a pre-treatment strategy is preferable to this approach. Consistent with these considerations, and given our findings of the benefits of angiogenic “pre-vascularization” of infarcted myocardium (Retuerto et al. “Angiogenic pre-treatment improves the efficacy of cellular cardiomyoplasty performed with fetal cardiomyocyte implantation” J. Thorac. Cardiovasc. Surg. 127:1 -11, 2004; incorporated herein by reference), and evidence that activation of anti-apoptotic mechanisms can also enhance the survival and functional benefit of cells implanted into myocardial scar (Fujio et al. “Akt promotes survival of cardiomyocytes in vitro and protects against ischemia reperfusion injury in the mouse heart” Circulation 101:660-67, 2000; incorporated herein by reference), we will utilize a VEGF pre-treatment strategy in a clinical trial provided the efficacy of this approach is confirmed in pre-clinical studies.

Autologous Skeletal Myoblasts

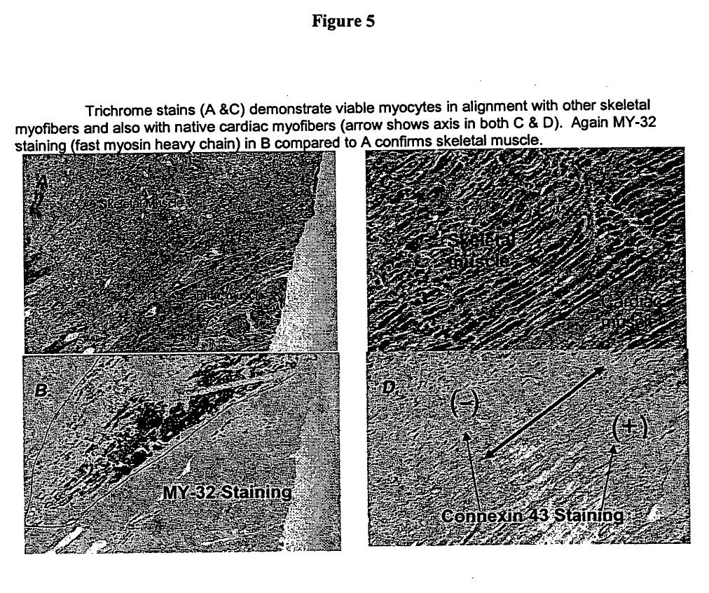

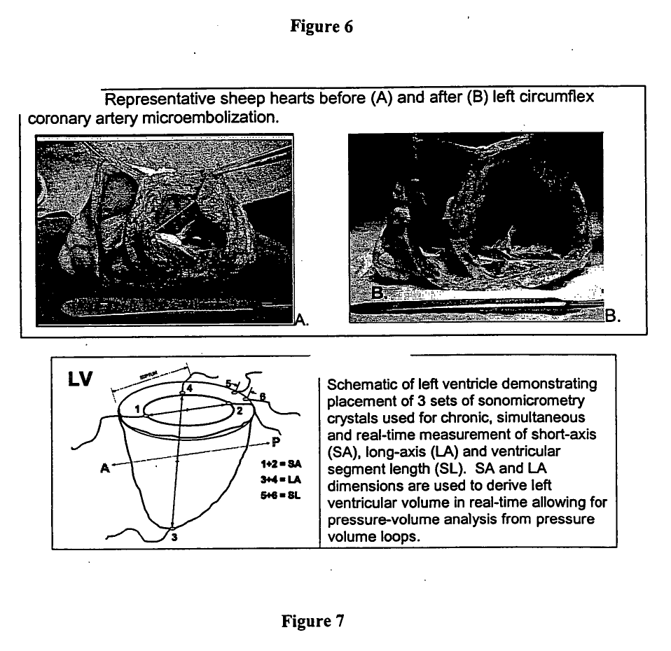

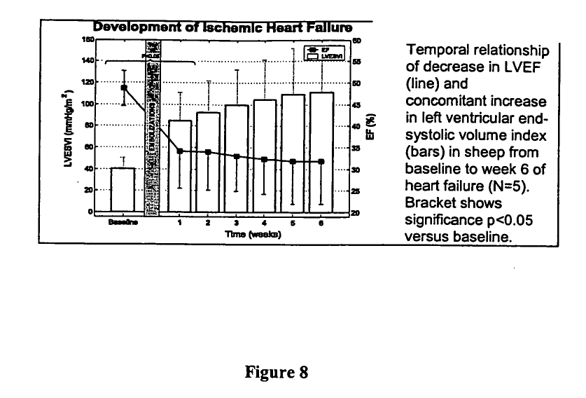

Animal studies in rodents and rabbits have demonstrated improved left ventricular performance (LVEF, developed pressures, preload recruitable stroke work), after skeletal myoblasts were transplanted into areas of myocardial scar (Taylor et al. “Regenerating functional myocardium: improved performance after skeletal myoblast transplantation” Nat. Med. 4(8):929-33, 1998; incorporated herein by reference). Ghostine and colleagues demonstrated in sheep transplanted with myoblasts after left circumflex coronary artery infarction that LV function was better than control animals (Ghostine et al. “Long-term efficacy of myoblast transplantation on regional structure and function after myocardial infarction” Circulation 106[suppl I]:I131-6, 2002; incorporated herein by reference). Skeletal myoblast transplantation attenuates the increase in LV volume (McConnell et. al. “Correlation of autologous skeletal myoblast survival with changes in left ventricular remodeling in dilated ischemic heart failure” J. Thorac. Cardiovasc. Surg. 2004 (in press); incorporated herein by reference; see FIG. 5) and improves regional myocardial wall motion scores and myocardial velocity gradients as determined by echocardiography and Tissue Doppler Imaging in infracted sheep at 4 months after myoblast injection (Ghostine et al. “Long-term efficacy of myoblast transplantation on regional structure and function after myocardial infarction” Circulation 106[suppl I]:I131-6, 2002; incorporated herein by reference).

Recent evidence in rats with fetal skeletal myoblasts, suggest that when sufficient numbers of myoblasts are transplanted into transmural scar, the LV geometry can be impacted in a beneficial manner (Tambara et al. “Transplanted skeletal myoblasts can fully replace the infarcted myocardium when they survive in the host in large numbers” Circulation 108[suppl II]:I1-259-63, 2003; incorporated herein by reference). Tambara et al (“Transplanted skeletal myoblasts can fully replace the infarcted myocardium when they survive in the host in large numbers” Circulation 108[suppl II]:II-259-63, 2003; incorporated herein by reference) demonstrated in a dose response fashion that ventricular dilatation (end-diastolic dimensions measured by echocardiography) was reversed with the injection of the highest number of transplanted cells (5.0×10⁷ cells). They also demonstrated by echocardiography, in a dose response fashion, the existence of late diastolic contractions of these larger volume cell implants, suggesting that resulting myofiber contraction may not be coupled to the cardiac cycle, but may be passive contraction or a ‘response to stretch’ at end-diastole (Taylor et al. “Regenerating functional myocardium: improved performance after skeletal myoblast transplantation” Nat. Med. 4(8):929-33, 1998; incorporated herein by reference). However, insufficient hemodynamic data exist in these smaller animal models to understand if the positive effects reported allow extrapolation to patients with severe end-stage CHF. Donor sources and the developmental origin (fetal) of the transplanted myoblasts in these studies further limit its clinical application since access to fetal tissues is problematic.

Human autologous skeletal myoblast (ASM) transplantation has been undertaken in both Europe and the United States. Menashe et al. (“Myoblast transplantation for heart failure” Lancet 357:279-80, 2001; incorporated herein by reference) in 2001 were the first to report ASM transplantation in a patient undergoing concomitant coronary artery bypass surgery (CABG). Transthoracic echocardiography (TTE) and Positron Emission Tomography (PET) demonstrated improved regional postoperative function in 14 of 22 previously scared myocardial segments (CABG with ASM injection). The first phase one trial in Europe (2003), reported on 10 patients with LVEF <35% who were evaluated and found to be candidates for coronary revascularization (Menasche et al. “Autologous skeletal myoblast transplantation for severe postinfarction left ventricular dysfunction” J. Am. Coll. Cardiol. 41:1078-83, 2003; incorporated herein by reference). These patients were concomitantly transplanted with approximately 870 million autologous skeletal myoblasts at the time of CABG. On average, there was an improvement in LVEF from 24% to 32% after ASM transplantation and CABG. Blinded echocardiographic assessment of regional wall function demonstrated improvement in 63% of implanted scars.

Three United States FDA Phase 1 multi-institutional, clinical trials (The Ohio State University, Arizona Heart Institute, University of California at Los Angeles, Temple University, Cleveland Clinic Foundation, Lindner Heart Research Center, University of Maryland, BryanLGH Heart Institute, and University of Michigan) also examined patients who underwent autologous skeletal myoblast transplantation and CABG or left ventricular assist device (LVAD) implantation (Dib et al. “Safety and feasibility of autologous myoblast transplantation in patients undergoing coronary artery bypass grafting: results from the United States experience” Circulation 106[suppl II]:II-463, 2002; incorporated herein by reference). Results from the U.S. Phase I trials also demonstrated significant reductions in NYHA class, improved myocardial perfusion and metabolic activity by PET, and increased myocardial wall thickness in 3 of 10 patients by cardiac MRI in patients undergoing concomitant CABG. Six patients underwent ASM injection at the time of left ventricular assist device (LVAD) implantation (Pagani et al. “Autologous skeletal myoblasts transplanted to ischemia-damaged myocardium in humans” J. Am. Coll. Cardiol. 41:879-888, 2003; incorporated herein by reference). Histological identification of viable skeletal myofibers was possible in four of five explanted hearts as far out as 191 days after injection and one explanted heart revealed histologic evidence of new vascular endothelium (CD 31) (Pagani et al. Autologous skeletal myoblasts transplanted to ischemia-damaged myocardium in humans. J. Am. Coll. Cardiol. 2003, 41: 879-888; incorporated herein by reference).

Stem Cells

Stem cells are defined as cells with self-renewal capability and the ability to transdifferentiate into multiple cell lineages (Verfaillie, “Adult stem cells: assessing the case for pluripotency” Trends Cell Biol. 12:502-508, 2002; Orkin et al. “Stem-cell competition” Nature 418:25-27, 2002; Anderson et al. “Can stem cells cross lineage boundaries?” Nat Med. 4:393-95, 2001; each of which is incorporated herein by reference). Stem cell therapy provides intriguing and exciting possibilities for the regeneration of myocardial scar; quite possibly in combination with myoblast precursors or alone if differentiation could be directed.

Recent research suggests that primitive stem cells within whole bone marrow possess great functional plasticity. After bone marrow transplantation, donor-derived stem cells have been found in such diverse nonhematopoietic tissues as skeletal muscle (Ferrari et al. “Muscle regeneration by bone marrow-derived myogenic progenitors” Science. 279:1528-30, 1998; each of which is incorporated herein by reference), cardiac muscle (Bittner et al. “Recruitment of bone-marrow-derived cells by skeletal and cardiac muscle in adult dystrophic mdx mice” Anat. Embryol. 199(5):391-6, 1999; incorporated herein by reference), liver bile ducts (Lagasse et al. “Purified hematopoietic stem cells can differentiate into hepatocytes in vivo” Nat. Med. 11:1229-34, 2000; Petersen “Hepatic ‘stem’ cells: coming full circle” Blood Cells Mol. Dis. 27(3):590-600, 2001; each of which is incorporated herein by reference), and vascular endothelium (Asahara et al. “Isolation of putative progenitor endothelial cells for angiogenesis” Science 275:964-67, 1997; Schatteman et al. “Blood-derived angioblasts accelerate blood-flow restoration in diabetic mice” J. Clin. Invest. 106:571-8, 2000; Asahara et al. “Bone marrow origin of endothelial progenitor cells responsible for postnatal vasculogenesis in physiological and pathological neovascularization” Circ. Res. 85(3):221-8, 1999; Shi et al. “Evidence for circulating bone marrow-derived endothelial cells” Blood 92(2):362-7, 1998; each of which is incorporated herein by reference). Using immunofluorescent techniques investigators have established that these bone marrow derived primitive cells had undergone the differentiation resulting in expression of markers specific for cardiomyocytes (Orlic et al. “Transplanted adult bone marrow cells repair myocardial infarcts in mice” Ann. NY Acad. Sci. 938:221-29, discussion 229-30, 2001; incorporated herein by reference). These results have been recently challenged by showing that bone marrow derived progenitor cells are subjected to cellular fusion with local cells. Therefore, these cells are prone to express a combination of markers including those characteristic for cardiomyocytes (Oh et al. “Cardiac progenitor cells from adult myocardium: homing, differentiation, and fusion after infarction” Proc. Natl. Acad. Sci. USA 100(21):12313-18, 2003; Terada et al. “Bone marrow cells adopt the phenotype of other cells by spontaneous cell fusion” Nature 416(6880):542-45, 2002; Yeh et al. “Transdifferentiation of human peripheral blood CD34+-enriched cell population into cardiomyocytes, endothelial cells, and smooth muscle cells in vivo” Circulation 108(17):2070-73, 2003; each of which is incorporated herein by reference).

Recently, more reliable genetic rather than immunofluorescence methods have been used to determine the final destination of pluripotential bone marrow derived progenitor cells at the site of myocardial injury. These experiments show that in a mouse model of myocardial injury, bone marrow derived progenitor cells undergo a low level transdifferentation directly into cardiomyocytes (Balsam et al. “Haematopoietic stem cells adopt mature haematopoietic fates in ischaemic myocardium” Nature 428(6983):668-73, 2004; incorporated herein by reference). Moreover, in the ischemic myocardium Lin⁻, c-kit⁺ population of transplanted cells was detected after 10 days, but by 30 days few cells were detectable and most of them expressed the hematopoietic marker CD45, suggesting that the final fate of transplanted progenitor cells was hematopoietic (Balsam et al. “Haematopoietic stem cells adopt mature haematopoietic fates in ischaemic myocardium” Nature 428(6983):668-73, 2004; incorporated herein by reference).

One of the most promising possibilities for the treatment of ischemic myocardium are cells known as endothelial precursor cells (EPC). EPC cells carrying phenotype markers CD34⁻CD133⁺CD7⁻lineage⁻ (lin⁻) are cells believed to be more primitive than CD34⁺ cells and could therefore, be endowed with nonhematopoietic potential and the ability to transdifferentiate into myogenic lineage (Gallacher et al. “Identification of novel circulating human embryonic blood stem cells” Blood 96(5):1740-47, 2000; incorporated herein by reference). Moreover, the infusion of endothelial progenitor cells with phenotypic and functional characteristics of embryonic hemangioblasts (CD34⁺, CD117⁺, VEGFR-2⁺, AC133⁺, GATA-2⁺) leads to direct induction of new vessel formation in the infarct area (vasculogenesis) and proliferation of preexisting blood vessels (angiogenesis) (Kocher et al. “Neovascularization of ischemic myocardium by human bone marrow derived angioblasts prevents cardiomyocyte apoptosis, reduces remodeling and improves cardiac function” Nature Medicine 7:430-436, 2001; incorporated herein by reference). Comparison of human skeletal myoblasts and CD133⁺ stem cells in a nude mouse acute infarct model demonstrated comparable results with respect to improvement in LV function (Agbulut et al. “Comparison of human skeletal myoblasts and bone marrow-derived CD133⁺ progenitors for the repair of infracted myocardium” JACC 44:458-463; incorporated herein by reference).

Unpurified mononuclear bone marrow cell suspensions contain large numbers of leukocytes and their progenitors may primarily induce local inflammation, rendering the actual stem cell effects insignificant. Instead, we will utilize a purified stem cell suspension using clinically available technology and methods that have been used in clinical trials related to cancer and heart disease. Two monoclonal antibodies are currently available for clinical selection of bone marrow stem cells, anti-CD34 and anti-CD133 antibodies. Cross reactivity of these antibodies with pig cells has been confirmed (Miltenyi Biotec, Chicago, Ill.), and preliminary data in our laboratory supports cross reactivity with sheep bone marrow cells. Whether these sheep cells represent immature lineage cells with transdifferentiation capability is currently work in progress.

Approximately 1% to 2% of CD34⁺ human bone marrow cells also express the CD133⁺ antigen, and 70-80% of CD133+ cells are CD34⁺. The CD133⁺ bone marrow cell population includes a small proportion of clonogenic cells, which have a very high potential to induce angiogenesis (Peichev et al. <<Expression of VEGFR-2 and AC133 by circulating human CD34(+) cells identifies a population of functional endothelial precursors” Blood 95:952-58, 2000; Reyes et al. “Origin of endothelial progenitors in human postnatal bone marrow” J. Clin. Invest. 109:337-46, 2002; each of which is incorporated herein by reference). There is accumulating evidence that the CD133⁺/CD34⁻ subpopulation includes multipotent stem cells with a potential for differentiation into mesenchymal and other non-hematopoietic lineages (Bhatia et al. “AC133 expression in human stem cells” Leukemia 15:1685-88, 2001; Kuci et al. “Identification of a novel class of human adherent CD34- stem cells that give rise to SCID-repopulating cells” Blood 101:869-76, 2003; each of which is incorporated herein by reference). Isolation of a purified CD133⁺ cell suspension is therefore, currently the most effective way to obtain a population of pluripotent adult stem cells in the clinical setting. The cell number achieved in clinical trials (up to 5 million CD133⁺ cells) may appear rather small compared with other cell types, but it should be remembered that this is a purified population of highly proliferative cells. In comparison, while other groups have used several hundred million unselected mononuclear bone marrow cells in clinical studies, less than 1% of those cells were potentially pluripotent stem cells. These observations support the use of CD133⁺ bone marrow cells.

Therapeutic Angiogenesis—Cell Based Strategies

Angiogenesis is the biologic process of new vessel formation that is a critical natural response to ischemia, and an important component of such pathologic processes as the vascularization of malignant neoplasms (Schott et al. “Growth factors and angiogenesis” Cardiovasc. Res. 27:1155-1161, 1993; Piek et al. “Collateral blood supply to the myocardium at risk in human myocardial infarction: a quantitative postmortem assessment” J. Am. Coll. Cardiol. 11:1290-129, 1988; Klagsburn et al. “Regulators of angiogenesis” Annu. Rev. Physiol. 53:217-23, 1991; Lee et al. “VEGF gene delivery to myocardial. Deleterious effects of unregulated expression” Circulation 102:898-901, 2000; each of which is incorporated herein by reference). The angiogenic molecules form a family of molecules known as vascular endothelial growth factors (VEGF) and basic fibroblast growth factors (Schott et al. “Growth factors and angiogenesis” Cardiovasc. Res. 27:1155-1161, 1993; incorporated herein by reference). Evidence that these mediators play a role in angiogenesis comes from several sources, including in vitro studies demonstrating mediator-induced endothelial cell proliferation, migration, and differentiation; examination of tissues demonstrating upregulation of these mediators and their relevant receptors in highly vascularized tissues and at sites of ischemia; the demonstration of neovascularization following in vivo administration of these mediators to ischemic tissues; and the suppression of aberrant neovascularization with the administration of molecules that inhibit these mediators and/or their function (Mack et al. “Biologic bypass with the use of adenovirus-mediated gene transfer of the complementary deoxyribonucleic acid for vascular endothelial growth factor 121 improves myocardial perfusion and function in the ischemic porcine heart” J. Thorac. Cardiovasc. Surg. 115:168-177, 1998; Schalch et al. “Adenoviral-mediated transfer of VEGF121 cDNA enhances myocardial perfusion and exercise performance in the non-ischemic state” J. Thorac. Cardiovasc. Surg. 127:535-540, 2004; Leotta et al. “Gene therapy utilizing adenovirus-mediated myocardial transfer of vascular endothelial growth factor 121 improves cardiac performance in a pacing model of congestive heart failure” J. Thorac. Cardiovasc. Surg. 123:1101-1113, 2002; Schalch et al. “Homozygous deletion of EGR-1 results in critical limb ischemia following vascular ligation: evidence for a central role for EGR-1 in vascular homeostasis” J. Thorac. Cardiovasc. Surg. 2004 (in press); Freedman et al. “Therapeutic angiogenesis for coronary artery disease” Ann. Intern. Med. 136:54-71, 2002; each of which is incorporated herein by reference).

Because this endogenous response to ischemia is often incomplete, the administration of exogenous growth factors, progenitor cells, or treated cells (Askari et al. “Cellular, but not direct, adenoviral delivery of vascular endothelial growth factor results in the improved left ventricular function and neovascularization in dilated ischemic cardiomyopathy” JACC 43:1908-14, 2004; incorporated herein by reference) known to induce angiogenesis has been used to “therapeutically” enhance the reperfusion of ischemic tissues (Mack et al. “Biologic bypass with the use of adenovirus-mediated gene transfer of the complementary deoxyribonucleic acid for vascular endothelial growth factor 121 improves myocardial perfusion and function in the ischemic porcine heart” J. Thorac. Cardiovasc. Surg. 115:168-177, 1998; Schalch et al. “Adenoviral-mediated transfer of VEGF 121 cDNA enhances myocardial perfusion and exercise performance in the non-ischemic state” J. Thorac. Cardiovasc. Surg. 127:535-540, 2004; Leotta et al. “Gene therapy utilizing adenovirus-mediated myocardial transfer of vascular endothelial growth factor 121 improves cardiac performance in a pacing model of congestive heart failure” J. Thorac. Cardiovasc. Surg. 123:1101-1113, 2002; Schalch et al. “Homozygous deletion of EGR-1 results in critical limb ischemia following vascular ligation: evidence for a central role for EGR-1 in vascular homeostasis” J. Thorac. Cardiovasc. Surg. 2004 (in press); Freedman et al. “Therapeutic angiogenesis for coronary artery disease” Ann. Intern. Med. 136:54-71, 2002; Suzuki et al. “Cell transplantation for the treatment of acute myocardial infarction using vascular endothelial growth factor-expressing skeletal myoblasts” Circulation 104[suppl I]:I-207-I-212, 2001; each of which is incorporated herein by reference). Furthermore, while the prolonged upregulation of growth factor expression induced by chronic gene transfer vectors has been shown to induce pathologic hemangioma formation (Lee et al. “VEGF gene delivery to myocardial. Deleterious effects of unregulated expression” Circulation 102:898-901, 2000; incorporated herein by reference), such has not been the case with transient growth factor expression, as provided by an adenovirus vector. In addition, VEGF pretreatment before an acute infarct followed by injection of myogenic precursors has been shown to enhance myoblast survival and translate into better functional outcomes in mice (Retuerto et al. “Angiogenic pre-treatment improves the efficacy of cellular cardiomyoplasty performed with fetal cardiomyocyte implantation” J. Thorac. Cardiovasc. Surg. 127:1-11, 2004; incorporated herein by reference).

VEGF

VEGF-A (VEGF) is the prototypical member of a family of structurally and functionally related polypeptides (Ferrara et al. “Molecular and biological properties of the vascular endothelial growth factor family of proteins” Endocr. Rev. 13:18-32, 1992; Houck et al. “The vascular endothelial growth factor family: identification of a fourth molecular species and characterization of alternative splicing of RNA “Mol. Endocrinol. 5:1806-1814, 1991; Leung et al. “Vascular endothelial growth factor is a secreted angiogenic mitogen” Science 246:1306-1309, 1989; Goto et al. “Synergistic effects of vascular endothelial growth factor and basis fibroblast growth factor on the proliferation and cord formation of bovine capillary endothelial cells within collagen gels” Lab. Invest. 69:50008-517, 1993; Dvorak et al. “Vascular permeability factor/vascular endothelial growth factor, microvascular hyperpermeability and angiogenesis” Am. J. Pathol. 146:1029-1039, 1995). VEGF is a heparin-binding glycoprotein encoded by a 14 kb, 8 exon gene that exists as at least four different species created by alternative splicing of a primary mRNA transcript. Of these, the 121 and 165 residue isoforms appear to be the most abundant and possess equivalent potency. VEGF is considered to be an endothelial cell-specific mitogen because of the nearly complete localization of its two high affinity tyrosine kinase receptors to this cell type (Fong et al. “Role of the flt-1 receptor tyrosine kinase in regulating the assembly of the vascular endothelium” Nature 376:66-70, 1995; incorporated herein by reference). Extensive data has linked VEGF and VEGF receptor expression with both normal biological and pathologic processes. Relevant to its role in tissue reperfusion, VEGF has been shown to be expressed by a variety of cell types, including cardiac myocytes and vascular smooth muscle cells, and has been shown to be transiently upregulated by ischemia through a specific oxygen/heme protein response element in the VEGF gene. The critical native activities of this potent angiogen, its relative endothelial selectivity (less selective mitogens pose the risk of fibrosis and/or intimal hyperplasia), its chemotactic properties, and the demonstrated ability of VEGF to potently induce angiogenesis in vivo underlie the use of VEGF in the current studies.

Gene Transfer

Gene transfer describes essentially a drug therapy that delivers a gene, a DNA sequence coding for a specific protein, to a host target cell that is thereby instructed to produce the protein of interest encoded by the transferred DNA sequence (Nabel et al. “Gene transfer and vascular disease” Cardiovasc. Res. 28:445-455, 1994; Imran et al. “Therapeutic angiogenesis: a biologic bypass” Cardiology 101:131-143, 2004; each of which is incorporated herein by reference). Relevant to the induction of angiogenesis, it has been demonstrated that a single dose of a gene transfer vector can provide growth factor expression for variable periods of time, depending on the gene transfer vector employed, of sufficient duration to induce neovasculature formation and enhanced perfusion (Mack et al. “Biologic bypass with the use of adenovirus-mediated gene transfer of the complementary deoxyribonucleic acid for vascular endothelial growth factor 121 improves myocardial perfusion and function in the ischemic porcine heart” J. Thorac. Cardiovasc. Surg. 115:168-177, 1998; Schalch et al. “Adenoviral-mediated transfer of VEGF121 cDNA enhances myocardial perfusion and exercise performance in the non-ischemic state” J. Thorac. Cardiovasc. Surg. 127:535-540, 2004; each of which is incorporated herein by reference). The great majority of angiogenic gene transfer studies in animals and humans have utilized plasmids or replication deficient adenoviruses (Ad) as gene transfer vectors. Constructs based upon adeno-associated viruses or retroviruses, with or without regulatable promoters, can provide prolonged transgene expression compared to Ad vectors, and may thus be of advantage for the current studies.

The adenoviruses are DNA viruses comprised of a 36 kb linear, double stranded DNA genome and core proteins surrounded by capsid proteins. Of the 49 human Ad serotypes, the subgroup C viruses, types 2 and 5, are the base for most gene transfer vectors. By deleting the E1 a sequence in the viral genome and inserting the exogenous gene of interest with an appropriate promoter, the virus can be made into a replication deficient vector capable of transferring the cDNA of interest to targeted cells or tissues. Adenovirus (Ad) vectors have properties that make them ideal for the delivery of VEGF genes for therapeutic angiogenesis. Ad vectors can be produced in high titer and are capable of efficiently transferring genetic information to replicating and non-replicating cells. Most importantly, Ad vectors are effective at transferring genes to cardiovascular tissues, with high levels of expression of the gene for at least one week (Mack et al. “Biologic bypass with the use of adenovirus-mediated gene transfer of the complementary deoxyribonucleic acid for vascular endothelial growth factor 121 improves myocardial perfusion and function in the ischemic porcine heart” J. Thorac. Cardiovasc. Surg. 115:168-177, 1998; incorporated herein by reference). This is a marked advantage compared to the very short half-life of the protein, but does not likely carry with it a risk of evoking too much angiogenesis in the target tissue, as might occur if the expression of the VEGF cDNA was long term (Lee et al. “VEGF gene delivery to myocardial. Deleterious effects of unregulated expression” Circulation 102:898-901, 2000; incorporated herein by reference). The new gene transferred by an Ad vector functions in an epi-chromosomal position, thus eliminating the risks of random insertional mutagenesis and permanent alteration of the genotype of the target cell. In contrast, adeno-associated virus and retrovirus vectors integrate the exogenous gene into the chromosome of the target cell, and thus carry the risk of inappropriately delivering the angiogenic stimulus long after it is needed.

Sustained, functionally significant stimulation of angiogenesis and tissue perfusion following Ad-mediated transfer of angiogenic genes has now been demonstrated in a number of relevant models, and has resulted in the execution of a number of Phase I/II clinical trials (Imran et al. “Therapeutic angiogenesis: a biologic bypass” Cardiology 101:131-143, 2004; Lee et al. “Exogenous control of cardiac gene therapy: evidence of regulated myocardial transgene expression after adenovirus transfer of expression cassettes containing corticosteroid response element promoters” J. Thorac. Cardiovasc. Surg. 118:26-35, 1999; Lee et al. “Focal angiogen therapy using intramyocardial delivery of an adenovirus vector coding for vascular endothelial growth factor 121” Ann. Thorac. Surg. 69:14-24, 2000; Rosengart et al. “Angiogenesis gene therapy: Phase I assessment of direct intramyocardial administration of an adenovirus vector expressing the VEGF121 cDNA to individuals with clinically significant severe coronary artery disease” Circulation 100:468-474, 1999; Rosengart et al. “Six-month assessment of a Phase I trial of angiogenic gene therapy for the treatment of coronary artery disease using direct intramyocardial administration of an adenovirus vector expressing the VEGF121 cDNA” Ann. Surg. 230:466-72, 1999; Magovem et al. “Direct in vivo gene transfer to canine myocardium utilizing a replication-deficient adenovirus vector” Ann. Thorac. Surg. 62:425-434, 1996; Magovem et al. “Regional angiogenesis induced in non-ischemic tissue by an adenovirus vector expressing vascular endothelial growth factor” Hum. Gene Ther. 8:215-227, 1997; each of which is incorporated herein by reference).

Use of Percutaneous Delivery of Cell Based Therapy to the Heart

Myocardial administration is the strategy proposed for the physical delivery of genetic information to myocardium. The most direct method of transferring genes to myocardium is by direct injection into the epicardium or endocardium. While surgical epicardial delivery of cells and gene transfer vectors affords many advantages such as direct visualization of the injection site and tangential delivery, a non-surgical approach may prove to be the best mode of delivery for the following reasons. First, catheter delivery has already been shown to lead to accurate delivery of cells and adenovectors to the myocardium (Grossman et al. “Incomplete Retention after Direct Myocardial Injection” Catheterization and Cardiovascular Interventions 55:392-397, 2002; Sanborn et al. “Percutaneous endocardial transfer and expression of genes to the myocardium utilizing fluoroscopic guidance” Cath. Cardiovasc. Diag. 52:260-266, 2001; Rutanen et al. “Adenoviral catheter-mediated intramyocardial gene transfer using the mature form of vascular endothelial growth factor-D induces transmural angiogenesis in porcine heart” Circulation 109:1029-35, 2004; Perin et al. “Transendocardial, autologous bone marrow cell transplantation for severe, chronic ischemic heart failure” Circulation 107:2294-2302, 2003; Chazaud et al. “Endoventricular porcine autologous myoblast transplantation can be successfully achieved with minor mechanical cell damage” Cardiovascular Research 58:444-450, 2003; Losordo et al. “Phase 1/2 placebo-controlled, double-blind, dose-escalating trial of myocardial vascular endothelial growth factor 2 gene transfer by catheter delivery in patients with chronic myocardial ischemia” Circulation 105:2012-18, 2002; Smits et al. “Catheter-based intramyocardial injection of autologous skeletal myoblasts as a primary treatment of ischemic heart failure: clinical experience with six-month follow-up” JACC 42:2063-2069, 2003; each of which is incorporated herein by reference). Secondly, a percutaneous approach will allow therapy to be given to patients who are not undergoing open chest surgery, thus broadening the patient population. Thirdly, a percutaneous route of delivery is expected to have lower morbidity than a surgical procedure. Lastly, catheter delivery of cells and vector in clinical studies will allow the inclusion of a blinded, placebo control group and permit a comparison of “sole” experimental intervention to placebo controls. To date, it has been difficult to discern the effects of cell or gene transfer therapies as adjuncts to CABG given the underlying improvements expected from CABG alone. Experiments proposed in the sheep heart failure model will directly compare epicardial and percutaneous endocardial injections.

Cordis/Biosense Catheter Technologies

The Cordis/Biosense catheter system with the NOGA™-guided MYOSTAR™ injection catheter will be used to deliver cells and adenovectors in pre-clinical studies comparing percutaneous versus surgical procedures. The Cordis/Biosense catheter creates a three-dimensional electromechanical map of the left ventricle and has a navigation system for endocardial delivery. To date, there are no injection catheters which are approved for use by the FDA. The Cordis/Biosense system has been chosen for the following four reasons. First, this catheter system has been used experimentally for intramyocardial delivery of stem cells, myoblasts, and AdVEGF gene transfer (Rutanen et al. “Adenoviral catheter-mediated intramyocardial gene transfer using the mature form of vascular endothelial growth factor-D induces transmural angiogenesis in porcine heart” Circulation 109:1029-35, 2004; Perin et al. “Transendocardial, autologous bone marrow cell transplantation for severe, chronic ischemic heart failure” Circulation 107:2294-2302, 2003; Chazaud et al. “Endoventricular porcine autologous myoblast transplantation can be successfully achieved with minor mechanical cell damage” Cardiovascular Res. 58 444-450, 2003; each of which is incorporated herein by reference). Second, the catheter has been proven to have a positive safety profile in clinical studies delivering myoblasts and gene transfer vectors (Losordo et al. “Phase 1/2 placebo-controlled, double-blind, dose-escalating trial of myocardial vascular endothelial growth factor 2 gene transfer by catheter delivery in patients with chronic myocardial ischemia” Circulation 105:2012-18, 2002; Smits et al. “Catheter-based intramyocardial injection of autologous skeletal myoblasts as a primary treatment of ischemic heart failure: clinical experience with six-month follow-up” JACC 42: 2063-2069, 2003; each of which is incorporated herein by reference). Third, by generating a three-dimensional map and superimposing the location of each endocardial injection, the dissemination of cells or vector in the target area can easily be achieved. Further, the chance of repeated dosing of the same location is minimized. Fourth, we have gained experience delivering pig myoblasts into infarcted animals. Our results support the choice of the Cordis/Biosense catheter system for safe percutaneous delivery into the myocardium.

Summary of background data. The current literature supports the use of cell implantation as a potential mean of improving ventricular function in the setting of chronic ischemic heart failure. Skeletal myoblasts and bone marrow stem cells, particularly CD133⁺ cells, may be ideal clinical candidates for this application, as is catheter deliver of these cells. Angiogenic pre-treatment of the ischemic/infarcted myocardium may enhance implanted cell survival, and thereby, the efficacy of cardiac cell transfer strategies in this setting. Together, these data support investigations of myoblasts and bone marrow stem cells in the proposed studies.

Preliminary Data on Percutaneous Biosense Cordis Injection Catheter®.

In order to demonstrate the feasibility and safety of delivery of myoblasts via percutaneous catheter delivery, a pig study was conducted. Initial in vitro studies were performed in which myoblasts were passed trough the MYOSTAR™ catheter system (Cordis/Biosense). The results indicated that passage through the catheter did not significantly alter the cell viability or density. In vivo testing involved six infarcted Yorkshire swine. Autologous skeletal muscle biopsies were obtained from the hind limb of each animal and expanded in vitro. Thirty days after infarction, cells were implanted into the scarred region of the myocardium. NOGA™ evaluation was performed directly before cell injection in order to generate a 3-D unipolar voltage map of the left ventricle and identify the area of infarction. An 8-F arterial sheath was used to advance the NOGA™-guided MYOSTAR™ injection catheter through the femoral artery. NOGA™ mapping also was used to guide and record the site of the injections. Two control animals were injected with transplantation media, two were injected with approximately 300×10⁶ myoblasts, and two animals were injected with approximately 600×10⁶ myoblasts. Sixty days post-transplantation, the swine hearts were harvested. To determine safety, animal well-being and survival, heart rhythm and comprehensive blood screening were evaluated over the 90-day study period. There were no deaths. Continuous rhythm monitoring using loop recorder revealed no arrhythmias, and no deaths or adverse events were recorded in any group during the 60-day period between transplantation and harvest. Nor were there significant differences in blood labs between groups. Myocardial function assessments revealed a trend toward improvement in the treatment groups with respect to ejection fraction, viability, as assessed by unipolar voltages, and cardiac index between transplantation and harvest. Histology of treated swine hearts identified no skeletal muscle myoblasts or myotubes, and indicated that lesions of treatment swine were not different from those in controls. These results are consistent with a large number of epicardial and endocardial injections of pig myoblasts performed in other studies (as opposed to successful engraftment in rats and sheep). In summary, the results indicate that percutaneous skeletal myoblast transplantation into an infarcted swine myocardium is feasible and safe using the NOGA™-guided MYOSTAR™ injection catheter, and may contribute to overall improved heart function.

In a separate arm of this study, one animal was injected with myoblasts which were labeled with iridium beads. Two hours after percutaneous catheter delivery of cells, the heart and tissue from the brain, kidneys, liver, lungs, and spleen were removed. Using neutron activated radioactive quantitation, we determined that approximately 4% of the cells were retained at the site of injection. No iridium-labeled cells were detected in the brain, kidney, or liver. Very low numbers of cells were detected in the spleen and in areas of the left ventricle not targeted for cell injection. The primary site outside of the heart where cells were detected was the lung which contained 5.1% of the injected cells.

Preliminary Data on Human Clinical Trials of Cell-based Therapy

To date, 30 patients have undergone autologous myoblast transplantation (10-300 million cells) in three clinical trials. Twenty four patients underwent coronary revascularization (average 2.7 grafts/patient) and autologous myoblast transplantation, and 6 patients underwent implantation of a left ventricular assist device (LVAD) at the time of autologous myoblast transplantation. An improvement in LVEF from approximately 28% prior to surgery to 35 and 37% was measured 12 and 18 months, respectively, following revascularization and autologous myoblast implantation. This improvement in LVEF correlated with a symptomatic improvement in NYHA classification of 2.1 to 1.5. In those patients eligible for Magnetic Resonance Imaging (MRI) follow-up, there was an increase in wall thickness in areas injected with myoblasts, indirectly suggesting improved perfusion and function. Several patients undergoing nuclear myocardial perfusion and Positron Emission Tomography (PET) studies demonstrated improved regional myocardial viability by glucose uptake without significant improvement in perfusion. The inference from this data is that the improved viability and lack of significant improvement in perfusion is likely the result of the implanted autologous skeletal myoblasts.

Histologic evidence of autologous myoblast survival and differentiation was confirmed in five of six evaluable LVAD recipients who subsequently underwent heart transplantation and pathologic examination of the transplanted heart (Trachiotis et al. “Coronary artery bypass grafting in patients with advanced left ventricular dysfunction” Ann. Thor. Surg. 66:1632-39, 1998; Tambara et al. “Transplanted skeletal myoblasts can fully replace the infarcted myocardium when they survive in the host in large numbers” Circulation 108[suppl II]:II-259-63, 2003; each of which is incorporated herein by reference). In summary, autologous myoblasts survived transplantation (positive staining for skeletal muscle-specific myosin heavy chain), autologous myoblasts differentiated into both myofibers and slow-twitch myosin isoforms (positive staining for myosin heavy chain beta), autologous myofibers aligned in parallel with host myocardial fibers, and in one patient there was an increase in the number of blood vessels (CD31⁺ staining) in the area of the grafted scar (Trachiotis et al. “Coronary artery bypass grafting in patients with advanced left ventricular dysfunction” Ann. Thor. Surg. 66:1632-39, 1998; incorporated herein by reference).

Five patients of the 24 undergoing concomitant CABG demonstrated non-sustained ventricular tachycardia (NSVT) with another patient requiring re-hospitalization and eventual AICD placement for inducible monomorphic ventricular tachycardia. The incidence of AICD placement has not appeared to be cell dose related as only 2 of 15 have required AICD placement in the highest dose group. In this patient, the recurrent ventricular tachycardia is likely to have been independent of myoblast transplantation based upon coronary angiography, coronary blood flow assessment, and electrophysiology studies demonstrating technical issues with the bypass graft to the anterior descending coronary artery, resulting in ischemia leading to inducible arrhythmia. One additional patient, who underwent planned AICD placement as part of a biventricular pacing regimen for CHF, had their device discharge twice at nine months after the surgery. No other AICD devices have discharged after placement.

Human clinical trials involving stem cell injections have reported improvement in LV function in the study population. However, the small number of patients, variability of cell preparation/origin, and concomitant revascularization confound the interpretation of these studies (Strauer et al. “Repair of infracted myocardium by autologous intracoronary bone marrow cell transplantation in humans” Circulation 106:1913-1918, 2002; Assmus et al. “Transplantation of progenitor cells and regeneration enhancement in acute myocardial infarction (TOPCARE-AMI)” Circulation 106:3009-17, 2002; Tse et al. “Angiogenesis in ischemic myocardium by intramyocardial autologous bone marrow mononuclear cell implantation” Lancet 361: 47-49, 2003; Perin et al. “Transendocardial, autologous bone marrow cell transplantation for severe, chronic ischemic heart failure” Circulation 107:2294-2302, 2003; Stamm et al. “Autologous bone-marrow stem-cell transplantation for myocardial regeneration” Lancet 361:45-46, 2003; Stamm et al. “CABG and bone marrow stem cell transplantation after myocardial infarction” Thorac. Cardiov. Surg. 52:152-8, 2004; each of which is incorporated herein by reference).

Summary of preliminary data. The studies described in this section demonstrate that sophisticated analyses of the diastolic vs. systolic mechanics of cell implantation are possible utilizing the ovine heart failure model established in our laboratory. These data are supportive of the currently proposed investigations of angiogenic pre-treatment of chronically ischemic hearts prior to cell implantation (myoblasts vs. bone marrow stem cells) as a potential means of treating ischemic heart failure in animal models, and, more importantly, in clinical trials.

Research Design and Methods

Hypothesis: Pretreatment with AdVEGF (AdVEGFₚᵣₑ) will promote improved cell survival and consequently function and geometry (contractility and/or remodeling) in sheep with chronic ischemic heart failure.

Protocol and Time Line (Table 4)

Cell survival, LV function, and LV remodeling after injection of autologous skeletal myoblasts or bone marrow-derived stem cells will be compared after AdVEGFₚᵣₑ or Null virus.

TABLE 4
ASM or BMSC with AdVEGFₚᵣₑ (N = 6/group × 5 groups)

| | Coronary | | | |
|---|---|---|---|---|
| | Microembolizations | AdVEGFₚᵣₑ or | 3 Weeks Later: | 8 Weeks Later: |
| | (Ischemic CHF | null or Saline | Cell or saline | Explant |
| Healthy | induction; | (thoracoscopic - | inject & chronic | heart & |
| animal | EF < 35%) | direct inject) | instrumentation | histology |
| Day 0 | Day 0-45 | Day 45-65 | Day 56-86 | Day >120 |

Group 1: AdVEGF + ASM
Group 2: AdVEGF + BMSC
Group 3: Null + ASM
Group 4: Null + BMSC
Group 5: True control (saline + media)

Methods:

Microembolization procedure-ischemic heart failure creation. Adult Dorsett sheep will be anesthetized and cared for during surgery as described herein. The left neck of each animal will be clipped and aseptically draped. Local anesthesia with 2% lidocaine.HCL mixed 1:1 with 0.5% Bupivacaine HCL (5 cc) will be injected into the skin and subcutaneous tissues where a 3 cm incision will be made for left carotid artery access. The left external carotid artery is exposed. A 5-0 prolene purse-string suture will be placed and a 5 or 7Fr arterial introducer (16-20 mm, Input.TS, Galway, Ireland) will be positioned using Seldinger technique (needle access, wire guided placement). The animal will be heparinized (5000 U Heparin) after introducer placement. Lidocaine (1 mg/kg i.v.) and magnesium sulfate (2 grams i.v.) will be given to the animals to reduce the risk of arrhythmias. A variety of coronary angiographic catheters and wires are available to obtain selective left circumflex coronary artery (LCXa) access for delivery of 0.5 cc to 1.5 cc of 70-100 μm polystyrene beads (Polysciences Inc., Warrington, Pa.). The first embolization will deliver 0.75 cc beads/50 Kg with all subsequent embolizations to deliver 1.25 cc beads/50 kg (McConnell et. al.” Correlation of autologous skeletal myoblast survival with changes in left ventricular remodeling in dilated ischemic heart failure” J. Thorac. Cardiovasc. Surg. 2004 (in press); incorporated herein by reference).

The wound will be closed in layers and dressed aseptically. Buprenorphine (0.3 to 0.6 mg i.m) will be used as needed for pain within 48 hours of each procedure. At the time of subsequent embolizations, the site of incision and arteriotomy will be made at increasingly more proximal positions on the neck.

Follow-up transthoracic echocardiography. Sheep will be lightly sedated with Telozol (1.5 mg/kg) as needed. Wool over the precordium and suprasternal notch will be shaved and the animals supported by a technician while either remaining standing or in a sling. Conventional two-dimensional transthoracic echocardiograms (TTE) will be obtained using a phased array GE Vivid 7 system (General Electric, Milwaukee, Wis.) equipped with a 2.5-3.0 MHz transducer. LVEF will be determined using GE Vivid 7 analysis work station/software and the area-length method at end systole and diastole. TTE will be performed 5 to 7 days after each microembolization procedure or weekly for 2 weeks if the LVEF is estimated to be <35% at the time of TTE. When the LVEF is determined to be <35% for two consecutive weeks, the following week the animal will undergo thoracoscopic AdVEGFₚᵣₑ injections.

Skeletal muscle biopsy. At the time of the first microembolization procedure, under the same anesthetic period, and prior to the left neck incision, a skeletal muscle biopsy (5-10 grams) will be harvested from the left forelimb. An area over the left forelimb will be clipped and aseptically draped in a separate sterile field. The forelimb muscle will be exposed and the biopsy taken using sharp dissection avoiding electrocautery and placed into a tube containing cell media for transport to the cell processing facilities for culture and expansion. Postoperative analgesia is the same as described for the combined microembolization procedure.

Bone marrow aspiration. During the general anesthetic period used for the final microembolization the sheep will undergo bilateral iliac crest bone marrow aspiration. A total of approximately 100-200 ml of bone marrow will be collected (75-100 ml per side). We have been able to achieve a total harvest of approximately 3.0×10⁶ cells. The bone marrow aspirate will then be transported to an on-site laboratory where cell processing will be completed.

Ad vector design, construction, and in vitro validation. The AdVEGF₁₂₁ vector to be used for these studies are standard replication deficient E1a⁻, partial E1b⁻, partial E3⁻ vectors proven functionally active and routinely utilized in other laboratories (Retuerto et al. “Angiogenic pre-treatment improves the efficacy of cellular cardiomyoplasty performed with fetal cardiomyocyte implantation” J. Thorac. Cardiovasc. Surg. 127:1-11, 2004; Mack et al. “Biologic bypass with the use of adenovirus-mediated gene transfer of the complementary deoxyribonucleic acid for vascular endothelial growth factor 121 improves myocardial perfusion and function in the ischemic porcine heart” J. Thorac. Cardiovasc. Surg. 115:168-177, 1998; Rosengart et al. “Angiogenesis gene therapy: Phase I assessment of direct intramyocardial administration of an adenovirus vector expressing the VEGF121 cDNA to individuals with clinically significant severe coronary artery disease” Circulation. 100:468-474, 1999; Rosengart et al. “Six-month assessment of a Phase I trial of angiogenic gene therapy for the treatment of coronary artery disease using direct intramyocardial administration of an adenovirus vector expressing the VEGF 121 cDNA” Ann. Surg. 230:466-72, 1999; Sanborn et al. “Percutaneous endocardial transfer and expression of genes to the myocardium utilizing fluoroscopic guidance” Cath. Cardiovasc. Diag. 52:260-266, 2001; each of which is incorporated herein by reference). Vector activity (pfu) tested on A293 cells and total particle units (pu) determined by spectrometry in final vector preparations typically yield pu/pfu ratios <30 and 1 RCA per 10¹⁰ pfu, while in vivo expression will be confirmed by ELISA of culture media from transduced cells, as previously described (Retuerto et al. “Angiogenic pre-treatment improves the efficacy of cellular cardiomyoplasty performed with fetal cardiomyocyte implantation” J. Thorac. Cardiovasc. Surg. 127:1-11, 2004; Mack et al. “Biologic bypass with the use of adenovirus-mediated gene transfer of the complementary deoxyribonucleic acid for vascular endothelial growth factor 121 improves myocardial perfusion and function in the ischemic porcine heart” J. Thorac. Cardiovasc. Surg. 115:168-177, 1998; Rosengart et al. “Angiogenesis gene therapy: Phase I assessment of direct intramyocardial administration of an adenovirus vector expressing the VEGF121 cDNA to individuals with clinically significant severe coronary artery disease” Circulation 100:468-474, 1999; Rosengart et al. “Six-month assessment of a Phase I trial of angiogenic gene therapy for the treatment of coronary artery disease using direct intramyocardial administration of an adenovirus vector expressing the VEGF121 cDNA” Ann. Surg. 230:466-72, 1999; Sanborn et al. “Percutaneous endocardial transfer and expression of genes to the myocardium utilizing fluoroscopic guidance” Cath. Cardiovasc. Diag. 52:260-266, 2001; each of which is incorporated herein by reference). Clinical lots of AdVEGF₁₂₁ will be characterized extensively for identity, safety, purity and quality.

Autologous skeletal myoblast tissue processing and culture. Approximately 5-10 grams of skeletal muscle will be obtained at each biopsy and subsequently stripped of connective tissue, minced into a slurry, and subjected to several cycles of enzymatic digestion at 37° C. with trypsin/EDTA (0.5 mg/ml trypsin, 0.53 mM EDTA; GibcoBRL) and collagenase (0.5 mg/ml; GibcoBRL) to release satellite cells. Skeletal myoblasts will be cultured according to a modified Ham's method (Asahara et al. “Isolation of putative progenitor endothelial cells for angiogenesis” Science 275:964-67, 1997; incorporated herein by reference). Satellite cells will be plated and grown in myoblast basal growth medium (SkBM; Clonetics) containing 15-20% FBS (Hyclone), recombinant human epidermal growth factor (rhEGF, 10 ng/mL), and dexamethasone (3 μg/mL). To prevent myotube formation during the culture process, cell densities will be maintained throughout the process so that <75% of the culture surface is occupied by cells.

All cells will be expanded for 11-12 doublings and will be cryopreserved prior to transplant. After thaw, myoblasts (as single cell suspension) will be washed and suspended in transplantation medium and a sample withdrawn to measure viability by Trypan Blue exclusion. After viability of the cell suspension is confirmed, cell concentration will be adjusted to approximately 150 to 300 million cells per cc and loaded into three to five 1 cc tuberculin syringes, chilled to 4° C. At the time of transplant, cells will be warmed to room temperature and injected without further manipulation. Viabilities for the cell suspension at the time of transplant will be measured. Myoblast purity will be measured by reactivity with anti-NCAM monoclonal Ab (5.1H11), using Flourescence Activated Cell Scanning (FACS). The antibody selectively stains myoblasts and not fibroblasts. All myoblasts extraction and expansion procedure will be performed at Core B.

Isolation of bone marrow stem cells (BMSC). Between 100 and 200 ml of sheep bone marrow will be aspirated into heparin-filled 20 ml syringes. Preparation of the bone marrow aspirate will be performed under hygienic conditions. Before and after every preparation step, cell samples will be drawn for determination of stem cell number, viability, and sterility. The mononuclear cell fraction will be isolated by Ficoll density centrifugation. Cells will be resuspended in phosphate buffered saline (PBS) containing 5% human serum albumin (HSA) and will be centrifuged again. The supernatant will be removed, the system will be refilled again with PBS/HSA, and cells washed a second time before they are resuspended in PBS/HSA. In the next step, monoclonal CD133 antibody conjugated to superparamagnetic ferrite crystals within a dextran-sacculus will be injected into the cell-processing bag and the suspension incubated for 30 minutes. After incubation, cells will be washed again with PBS/HSA. The cell-processing bag will be removed from the processing system, and the cells resuspended with PBS/HSA in a transfer bag. The transfer bag will be connected to the CliniMACS Magnetic Cell Separation device (Miltenyi Biotech, Bergisch Gladbach, Germany). Inside the CliniMACS system, the cells run through an iron matrix-filled column, which is placed inside a strong permanent magnet. Cells bound to the ferrite crystal-conjugated AC133 antibody are retained within the column, while unlabelled cells pass through and are collected in a waste bag. After removal of the magnetic field the CD133⁺ cells will be washed out of the column and the procedure repeated twice, yielding a purified CD133⁺ cell suspension. After calculation of the number of viable stem cells, cells will be centrifuged for 10 minutes, resuspended in PBS/SSA, and adjusted to a cell concentration of 0.5×10⁶ cells/ml to 2.5×10⁶ cells/ml, respectively. The cells will be aliquoted into 2 ml vials, resulting in final dosages of 1.0×10⁶ to 5×10⁶ target cells per vial. The stem cells will be filled into pre-sterilized 2 ml plastic tubes and packed in a sterile container.

Thoracoscopic direct injection (transepicardial) of AdVEGFₚᵣₑ and cells. After heart failure induction but before cell injections, adenovirus containing VEGF will be directly injected into the scarred myocardium by syringe technique. Dorsett sheep will be anesthetized and cared for during surgery as described herein. The right chest of the sheep will be shaved to facilitate aseptic membrane, Ioban (3M, St Paul, Minn.), placement as part of the surgical drape to reduce infection risk. Each animal will be administered lidocaine HCL mixed 1:1 with bupivacaine HCL subcutaneously prior to incision and then for intercostal nerve block through which the thoracoscopic ports will be placed. Under single lung ventilation, four endoscopic ports (Ethicon, Cincinnati, Ohio; 2×12 mm and 2×5 mm) will be placed into the right chest and carbon dioxide insufflation (5-8 mmHg) will be used to augment visualization. A 10 mm endoscope (Stryker Endoscopy, San Jose, Calif.) will be passed into the chest, and a pericardiotomy will be created and pericardial cradle fashioned by passing a 2-0 silk suture on a Keith needle intercostally and temporarily secured at the chest wall. A flexible laparoscopic liver retractor will be used to apply slight and gentle traction to the right side of the heart exposing the posterolateral LV.

Through a 5 mm port, the syringe needle will be introduced into the thoracic cavity and directed to the area of myocardial scar. The technique includes passing a flexible 26 gauge round tip spinal needle (Monoject: 230539, St. Louis, Mo.) into the mid-myocardium at a shallow angle in the mid-myocardium (parallel with the circumferential axis of the heart) to a linear pass depth of approximately 3-4 cm. As the needle is withdrawn, the AdVEGF will be injected. The administration of approximately ten uniformly distributed 20 μL injections each containing 2×10⁹ pu (2×10¹⁰ total dose) of AdVEGF or adenovirus with an empty expression cassette (AdNull) prepared as described above.

Trocars will be removed from the chest, the animal converted to double lung ventilation, incisions closed in layers, the pneumothorax evacuated using a 16Fr chest tube through prior 5 mm port site, and the animal recovered. Post-operative care will be as described herein.

Surgical preparation/chronic instrumentation. Dorsett sheep will be cared for during surgery as described herein. Using strict aseptic technique, a left thoracotomy will be made, and pericardium opened and instrumented as follows: Six sonomicrometry crystals (2 mm, Sonometrics, London, Ontario) will be placed on the endocardial surfaces and in the mid-myocardium (segment length) in the configuration illustrated in FIG. 7 and secured using sutures. A dual pressure telemetry unit (Model #: TL11M3-D70-PCP, DSI, St. Paul, Minn.) will provide both aortic and left ventricular pressure catheters. These will be placed into the descending thoracic aorta and LV apex, respectively. The catheters will be passed through the thoracotomy, and the device will be secured in a subcutaneous pocket on the left chest. A 16 mm inferior vena cava (IVC) occluder (In Vivo Metrics, Healdsburg, CA) will be positioned around the intrathoracic IVC and secured. A right ventricular (RV) fluid filled catheter will be positioned in the RV. The cell-based therapy injections will take place after instrument implantation but prior to exiting catheters and instruments. The chest will be closed in layers, the wound dressed aseptically, and the animal fitted with a soft jacket. Post operative analgesia will be as described herein.

Cellular injections (ASM or BMSC). Autologous skeletal myoblasts or stem cells will be made available to the operating surgeon in 1 or 2 sterile 3 ml syringes. The cells or cell media (controls) will be injected into the infarcted myocardium at sites that were previously injected with AdVEGF. Specifically, 0.2 ml of cells will be injected at each of approximately ten sites. The technique includes passing a flexible 26 gauge round tip spinal needle (Monoject: 230539, St. Louis, Mo.) into the mid-myocardium at a shallow angle in the mid-myocardium (parallel with the circumferential axis of the heart) to a linear pass depth of approximately 3-4 cm. As the needle is withdrawn the cells will be injected.

Hemodynamic and ECG monitoring. Animals will be placed into an open large animal transport. The sheep will be able to stand upright without restriction and will not require restraint to perform studies. The animal will have the telemetry unit activated using a small magnet. A RMC-1 receiver (DSI) is hung inside the animal transport, in close and unrestricted proximity to the sheep. A UA-10 digital to analog converter will be used to send calibrated signals to an 8-channel data acquisition system (EMKA) where ECG and hemodynamic waveform analysis will be completed using cardiovascular software (IOX, EMKA). Both aortic and LV pressure signals will be analyzed for standard hemodynamic indices to include but will not be limited to: HR, SBP, DBP, MAP, LVESP, LVEDP, dP/dT max and at 40 mmHg, Tau, contractility index, and developed pressure. The ECG waveforms will be collected via telemetry overnight (12 hours) at 72 hour intervals and analyzed in each animal for evidence of arrhythmias (atrial or ventricular). Right ventricular pressures will be collected via fluid filled catheters to a calibrated Statham pressure transducer that is connected to a signal amplifier (Gould).

Random 24-hour ECG monitoring. Each animal will be monitored for 24 hours to assess cardiac arrhythmias for the first 24 hours after ASM injection and then overnight on average every 3 days in a random fashion. Each sheep will have its telemetry device (DSI) activated using a small magnet and the RMC-1 receiver will be placed inside the housing run and set up as described above.

Sonomicrometry and Pressure-Volume analysis protocol. Sonomicrometry skin button (Sonometrics) will be connected to a 6 channel TRX Series 4 receiver (Sonometrics) and passed into analysis software (Sonoview, Sonometrics) and then sent through a 4 channel digital to analog converter (Sonometrics) to an 8-channel data acquisition and analysis system (IOX, EMKA) where the signals will be calibrated. Sonomicrometry signals for long axis (LA), short axis (SA), and segment length (SL) will be individually analyzed by the software for waveform independent (minimum, maximum, mean, etc.) and cardiac-cycle dependent (end-diastolic and end-systolic) measures. Volume will be calculated in real-time from signals for SA and LA and then calculated with the equation for an ellipse (SA²*LA*π/6)/1000(mL). See section on hemodynamic monitoring for telemetered LV pressure acquisition. Respective pressure and volume signals will be brought into the software in sync and pressure-volume (PV) and pressure-distance loops will be generated. Off-line PV analysis will be completed with IOX software. Eₑₛ, E_{ed}, PRSW, and Eₘₐₓ (maximal time varied elastance) and respective regression analyses will be performed.

Five minutes of baseline hemodynamic data will be aquired (signals: Aortic, LV, ECG, SA, LA, SL, calculated LV volume). IVC occlusions will be carried out for generation of PV relationships. A typical occlusion will be less than 10 seconds in duration, and the animal will be allowed to recover for approximately 2 minutes prior to subsequent occlusions. Two to three occlusions will be performed per animal per intervention (Dobutamine dose response).

Dobutamine dose responses. To better define impact of cell injection on LV function, we will collect data at baseline and after three increasing doses of dobutamine. The RV catheter will provide central venous access for dobutamine administration. A perfusion pump (Baxter, model AS20GH-2, Hooksett, NH) with dobutamine (0.125 mg/cc) will be programmed to deliver 1 μg/kg/min, 2.5 μg/kg/min, and 5 μg/kg/min doses. The animal will be allowed to stabilize for 2 minutes at each dose. Baseline data (1 minute) will be collected and then 2 IVC occlusions performed with 1 minute for stabilization between occlusions. This protocol will be repeated for each dose at weekly intervals.

Histology. Eight (8) weeks after ASM injection, the animal will be euthanized using a lethal dose of supersaturated KCL while under deep thiopental anesthesia (40 mg/kg, i.v.). The heart will be quickly removed and fresh tissue biopsies (5 grams) taken and frozen at −70° C. for later analysis (see below). The heart will then be perfused with a 10% buffered formalin solution and stored in formalin for at least 24 hours before tissue processing. Tissue blocks will be made from 1) areas of remote myocardium (non-infarcted), 2) embolized myocardium that did not receive ASM, and 3) from embolized myocardium receiving cell treatments. Tissue blocks will then be embedded in paraffin and 5 μm sections cut. Histochemical and immunohistochemical stains will be performed in order to characterize graft survival and differentiation of injected sheep myoblasts. Sections will be stained separately with Hematoxylin & Eosin, and Trichrome using standard methods.

To confirm the phenotype of the mature grafts more than 28 days after engraftment, deparaffinized sections will be stained immunohistochemically with an anti-myosin heavy chain antibody that does not react with cardiac muscle, alkaline phosphatase-conjugated MY-32 mAb (Sigma). Sections will be developed with BCIP-NBT (Zymed Lab Inc) and counter stained with nuclear red. Additionally stains for connexin-43 Ab (Mouse monoclonal, IgGl, Chemicon, Temecula, Calif. Catalog number MAB3068) will be performed.

Estimation of Myoblast Survival. As described above, tissue sections will be stained with H&E, or Trichrome and immunostained for skeletal-specific myosin heavy chain (MY32), myogenin, or myoD. To approximate the survival of myoblasts in the heart, we will determine the area of the graft(s) in a representative tissue section, the density of nuclei per graft area, and use the following equation to determine the total number of surviving myoblast nuclei in the tissue block.

$$\begin{matrix}Sum of Graft \\ Area in Section\end{matrix} \times \begin{matrix}Density of Nuclei \\ Per Graft Area\end{matrix} \times \begin{matrix}\# Sections \\ Per Block\end{matrix} \times \begin{matrix}Abercrombie \\ {Correction}^{*}\end{matrix}$$

$${}^{*} The Abercrombie correction adjusts for the possibility of counting the same nucleus in adjacent sections .$$

Estimation of CD133⁺ cell survival and differentiation. Gene transfer provides an alternative and potentially a superior approach to monitor the fate of transplanted stem cells. Reporter genes expressing Enhanced Green Fluorescence Protein (EGFP) have been used to follow the fate of myocytes and myogenic stem cells (Gepstein et al. “A novel method for nonfluoroscopic catheter-based electroanatomical mapping of the heart: in vitro and in vivo accuracy results” Circulation 95:1611-1622, 1997; Roell et al. “Cellular cardiomyoplasty improves survival after myocardial injury” Circulation 105:2435-2441, 2002; Muller et al. “Selection of ventricular-like cardiomyocytes from ES cells in vitro” FASEB J. 14:2540-2548, 2000; each of which is incorporated herein by reference). Also, EGFP is compatible with a variety of imaging techniques, and as such might be useful to monitor the transplanted cells in the heart. Therefore, we will be using this approach to monitor the cells after transplantation. The stem cells derived from bone marrow or the skeletal muscle cells will be expanded or cultured, and the cells will be transduced with a vector encoding an enhanced green fluorescent protein (EGFP) marker gene. GFP labeled CD133⁺ cells will be co-stained with tissue specific antigens (connexin 43 [cardiac], CD45 [haematopoietic], GR-1 [myeloid], CD31 [endothelial]) to verify the presence of injected CD133⁺ cells within the scarred myocardium and their transdifferentiation.

Estimation of neovascularization. To quantitate capillary density the infarct tissue sections will be stained with monoclonal antibodies for CD31, factor VIII and major histocompatability complex (MIC) as described by Schuster et al. (Schuster et al. “Myocardial neovascularization by bone marrow angioblast results in cardiomyocyte regeneration” Am. J. Physiol. Heart Cir. 287:H525-H532, 2004; incorporated herein by reference) and compared with capillary density of the unimpaired region of the heart. Values are expressed as the number of CD31⁺ capillaries per high power field (HPF).

Statistical Analysis. A power analysis was performed in order to determine the number of sheep required to demonstrate a statistical difference in the short-axis length between controls and the ASM groups, under the assumption that the observed differences in the preliminary data (between control and low/high survival ASM) will extrapolate to those in the proposed groups. The preliminary studies described above suggest that ASM injection attenuates SA dilatation by 6.9% (σ=4.05%) when compared to control. Similar differences in attenuation were observed between low and high cell survival groups (6.6%, σ=3.62%). Assuming the wider variance, at least 6 animals per group are required to detect a difference of 6.6% (minimum) between the five groups at the p<0.05 level with 80% probability (α=0.05, β=0.80). Hence, a total of 30 sheep would be required to complete the work described in Table 4. Considering loss of animals either as a result of microembolization, instrumentation, and/or cell therapy in HF animals (˜30% animal loss), the projected total number of animals to start the study would be 40 sheep.

Hemodynamic, geometrical, and functional data will be studied using multi-factorial analysis of variance (ANOVA) with repeated measurements design. For example, a comparison of the differences in LV dilatation (either in SA, LA, or LV volume) or function (LVEF, Eₑₛ, LV segment shortening) will be evaluated using a two factor mixed design with repeated measures: (Table 4 Groups 1-5) and at two time points (1 week and 8 weeks post-cell inject). If the F ratio is found to exceed a critical value (p<0.05), then the significance of the differences between means will be tested using the Bonferroni's post-hoc test.

Interpretation of results. The current major limitation of cell-based therapy is the inadequate survival of cell grafts (Pagani et al. “Autologous skeletal myoblasts transplanted to ischemia-damaged myocardium in humans” J. Am. Coll. Cardiol. 41: 879-888, 2003), this in part, may be due to the poor oxygen and nutrient supply of the recipient tissue-in this case, chronic myocardial scar in end-stage CHF.

We propose to study the effect of pretreatment of myocardial scar with vascular endothelial growth factor-121 via adenoviral vector (AdVEGFₚᵣₑ), a potent stimulant to neovascular growth as demonstrated in our preliminary studies (Retuerto et al. “Angiogenic pre-treatment improves the efficacy of cellular cardiomyoplasty performed with fetal cardiomyocyte implantation” J. Thorac. Cardiovasc. Surg. 127:1-11, 2004) and the work of others (Askari et al. “Cellular, but not direct, adenoviral delivery of vascular endothelial growth factor results in the improved left ventricular function and neovascularization in dilated ischemic cardiomyopathy” JACC 43:1908-14, 2004; Suzuki et al. “Cell transplantation for the treatment of acute myocardial infarction using vascular endothelial growth factor-expressing skeletal myoblasts” Circulation 104[suppl I]:I-207-I-212, 2001, each of which is incorporated herein by reference). Also in favor of angiogenic pretreatment as based on preliminary data (Retuerto et al. “Angiogenic pre-treatment improves the efficacy of cellular cardiomyoplasty performed with fetal cardiomyocyte implantation” J. Thorac. Cardiovasc. Surg. 2004; 127:1-11, 2004; incorporated herein by reference), we believe that pretreatment is necessary to have appropriate neovascular formation in place at the time of cell injection, as this early period likely represents a critical time for cell retention and survival. We have chosen to directly, though minimally invasively (thoracoscopically), transepicardially inject sheep with prior infarcts and heart failure with AdVEGFₚᵣₑ 3 weeks prior to direct surgical transepicardial cell injection. If thoracoscopic procedures in any way compromiseaccess to the myocardium for injections, then a minimally invasive open technique can and will be employed.

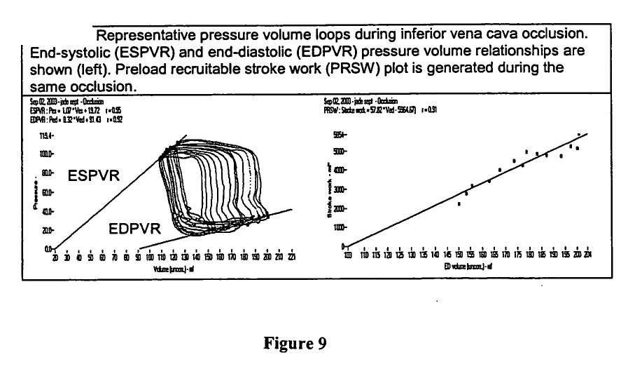

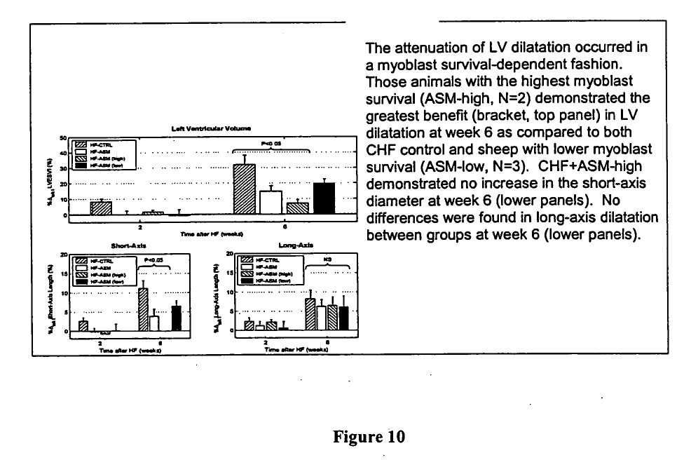

The primary goals are to determine 1) can AdVEGFₚᵣₑ improve cell survival?, and 2) is there functional (LV contractility or remodeling) advantage to AdVEGF_{pre.} +cells. Furthermore, we have chosen to treat myocardial scar with the VEGF₁₂₁ delivered via adenovirus rather than transfecting cells with this protein. In our prior studies, we have seen positive effects of ASM on LV remodeling as early as 3-4 weeks after injection and have identified skeletal myofibers beyond six weeks (FIGS. 27 and 22, respectively), therefore we will address the end-points of 1) cell survival, 2) LV function, and 3) LV remodeling by studying these animals for up to 8 weeks after cell injection. The treatment arms have been adequately weighted to account for changes in LV remodeling as found in our preliminary studies (FIG. 9). Exclusion of an ASM alone group or null virus alone group is justified based on preliminary data using ASM alone and the fact that null virus pretreatment/cell media will provide an appropriate control.

Research Design:

Hypothesis. It is our hypothesis that percutaneous delivery of optimum cell-based therapy (ASM or BMSC) will be effective in animals with ischemic dilated heart failure.

Protocol and Timeline (Table 5)

Efficacy of using the Biosense Cordis catheter for endocardial delivery will be compared to that of epicardial myocardial syringe injection strategies. An evaluation of percutaneous delivery AdVEGFpre will be compared to direct epicardial methods. In addition to ECG and basic hemodynamic data, cell survival, LV function, and LV remodeling after injection of ASM or BMSC will be compared to appropriate groups.

TABLE 5
Percutaneous Transendocardial Delivery of AdVEGFpre + ASM or BMSC
cells (Optimal Therapy) via Biosense Cordis Catheter in sheep with CHF.
(N = 6/group × 2 groups)

| | | | | 3 Weeks | |
|---|---|---|---|---|---|
| | Chronic | | | Later: | |
| | Instrumentation | | AdVEGFₚᵣₑ | Percutaneous | |
| | (Telemetered | Coronary | or null | Cell injection | 8 Weeks |
| Healthy | LVP, AoP and | Microembolization | (Biosense ®) | (Biosense ® | Later: |
| animal | ECG) | (LVEF <35%) | Catheter) | Catheter) | Histology |
| Day 0 | Day 0-14 | Day 14-60 | Day 60-80 | Day 81-101 | Day >140 |

Methods:

Surgical preparation/chronic instrumentation. Surgical drape and anesthesia will be as described herein, but in healthy sheep. A dual pressure telemetry unit (Model number: TL11M3-D70-PCP, DSI, St. Paul, Minn.) will provide both aortic and left ventricular pressures. These catheters will be placed into the descending thoracic aorta and LV apex, respectively. Biopotential leads will be placed subcutaneously cephalad and caudal to the heart for recording of single lead ECG. The catheters will be passed through the thoracotomy, and the device will be secured in a subcutaneous pocket on the left chest.

CHF Model and cell therapy preparation. Left circumflex coronary microembolization, transthoracic echo for determination of heart failure, skeletal muscle biopsy, bone marrow biopsy, Ad vector production, autologous myoblast processing, and the isolation of stem cells from bone marrow will be performed.

NOGA-™ mapping in sheep. Each animal will be anesthetized and cared for during surgery as described herein. An adhesive reference patch will be placed on the right side of the animal, over the 3-5 intercostal spaces. Under fluoroscopic guidance to the descending thoracic aorta via the left carotid artery (8Fr sheath), the mapping catheter will be deflected to form a J shape and will be introduced across the aortic valve into the left ventricle. The location of the catheter will be gated to the end of diastole and recorded relative to the location of the fixed reference catheter at that time. As the catheter tip is moved over the LV endocardial surface, the system analyzes its location in 3-dimensional space without the use of fluoroscopy. Results will be collected from both unipolar (UP) and bipolar (BP) recordings filtered at 0.5 to 400 Hz. The stability of the catheter-to-wall contact will be evaluated at every site in real time.

All maps will be acquired with an interpolation threshold of 15 mm between adjacent points. The 3-dimensional LV endocardial reconstruction is updated in real time with the acquisition of each new site and displayed continuously on a Silicon Graphics workstation. Myocardial areas that manifest both high electrical signals (UP endocardial voltage 10 mV and BP endocardial voltage 2 mV) and normal LS (TLS <80% and LLS>12%) will be interpreted to represent normal myocardial function. Areas with impaired electrical activity (UP voltage <10 mV and BP voltage <2 mV ) and impaired mechanical function (TLS >80% and LLS <8%) will be interpreted to represent abnormal electromechanical properties in areas of MI.

Intra-myocardial catheter injection procedure. Insertion of an introducer sheath of at least 8 F will be performed into the right or left femoral artery using standard procedures for percutaneous coronary angioplasty. After administration of 5000 units heparin.HCl, the following will be performed:
baseline left ventriculography in standard views to assist with guidance of the catheter;
baseline electromechanical map using the mapping catheter to assist with guidance of injections;
after endocardial mapping, an 8 F injection catheter will be placed via the carotid sheath into the LV;
orientation of the injector catheter (incorporating an EM tip sensor) to the treatment zone (infarcted area of the heart muscle), using the baseline electromechanical map and fluoroscopic guidance;
establish the stability of the injection catheter on the endocardial surface (according to the recording of loop-stability value <2 and cycle length stability during sinus rhythm);
extend the injection needle into the myocardium to a depth of approximately 4-6 mm, adjusted to wall thickness. Injections will be administered in a volume of 0.20 ml and spaced ˜1 cm apart;
repeat injections have been made in a distribution into the center and around the area of the infarct;
the density of injection sites will depend upon LV endocardial anatomy and the ability to achieve a stable position on the endocardial surface without catheter displacement or PVCs. The workstation software will provide precise annotation of the location in 3-dimensional (3-D) space for each injection site; and
the injection catheter will be removed at the conclusion of the endomyocardial injections.

Adverse events (hypotension, cardiac depression [diminished dP/dT], and/or rhythm) will be monitored via chronic telemetry catheters and leads. Cardiac enzymes (tropinin I and CKMB) will be drawn at 12-24 hours.

Weekly echocardiography. Sheep will be lightly sedated with Telozol (1.5 mg/kg) as needed. Wool over the pre-cordium and suprasternal notch will be clipped and the animals supported by a technician while either remaining standing or in a sling. 2D and M-mode transthoracic images will be obtained with a 2.5 and/or 3.0 MHz dual frequency transthoracic transducer. Long and short-axis views will be obtained at rest with animal standing in a large animal stanchion designed for access to either side of the sheep's thorax. Regional wall thickening, ventricular dimensions, fractional area change, ejection fraction, and tissue Doppler analyses (TDI) of infarct border, infarct+cell therapy and remote myocardium will be studied. LVEF and TDI will be determined using standard processing in a GE Vivid 7 analysis work station.

Statistical analysis. A total of 12 sheep (N=6/group) will be required to complete the work (Table 5). Considering loss of animals either as a result of surgeries and/or cell therapy in HF animals (˜30% animal loss), the projected total number of animals will be ˜16.

Interpretation of results. The primary goal of the work (Table 5) is to evaluate the efficacy of cell-based therapy using percutaneous endocardial injection. The study endpoints will be the evaluation of cardiac function and remodeling (weekly echo and chronic telemetered hemodynamics) after percutaneous endocardial injection compared to direct or epicardial injection of cell therapy. The results of these studies will lead us to recommend treatment strategies for clinical trials. The use of the same catheter for cell and AdVEGF will also allow us to evaluate the safety of the catheter in significantly more animals prior to clinical use.

Sonomicrometry and other elaborate instrumentation will be avoided in this group to more accurately represent the non-surgical CHF patient. We will compare improvements in segmental and global LV function obtained from weekly echocardiographic studies (TDI and WMS) to sonomicrometry data. Again, this will allow us to directly compare the relative efficacy of percutaneous endocardial delivery of cells with direct epicardial delivery of cells.

Limitations and Alternatives

Sheep possess similar cardiac and coronary anatomy to that of humans (Huang et al. “Remodeling of the chronic severly failing ischemic sheep heart after coronary microembolization: functional, energetic, structural and cellular responses” Am. J. Physiol. 286:H2141-50, 2004; incorporated herein by reference). The coronary microembolization model has been well studied in sheep as well as other species and closely resembles multi-infarct human pathology leading to dilated ischemic heart failure (Huang et al. “Remodeling of the chronic severly failing ischemic sheep heart after coronary microembolization: functional, energetic, structural and cellular responses” Am. J. Physiol. 286:H2141-50, 2004; incorporated herein by reference). The restriction to the left circumflex coronary artery, though only representative of a fraction of patients with isolated disease of this artery, allows for better survival in these experimental animals since the incidence of fatal arrhythmia is less when avoiding septal perforators (off the left anterior descending artery) are avoided. However, inherent differences between this selective process versus the less selective human disease could confound findings, but control studies should help to minimize these discrepancies. Mitral regurgitation results after LV dilatation has progressed, as with patients who undergo substantial LV dilatation.

The chronic hemodynamic studies demonstrated in our preliminary results and proposed in these future studies are complex in that animal welfare can significantly impact physiology. We have a policy of daily monitoring our animals for infections and other instrument related complications that has resulted in not a single animal being lost to infection. All unanticipated loss of animals from preliminary studies has been during the microembolization procedure (ventricular arrhythmias or acute heart failure, ˜75% of loss) or perioperatively (at anesthetic induction, during or within several hours of the procedure, ˜25% of loss) due to the thoracotomy while the animal is in heart failure (LVEF<35%). Total loss has been ˜30% (14/48 sheep) of those animals starting like study protocols, and this percent loss has been factored into the study design/statistics.

Delivery of AdVEGFₚᵣₑ using thoracoscope, though not previously presented in the literature utilizes a technique that we have experience with in patients (lateral pacing lead placements thoracoscopically) and have used in animal models of total endoscopic coronary artery bypass (unpublished studies and training sessions on the Da Vinci Robot in the Cardiothoracic Surgery laboratory at Ohio State Medical Center). We feel that using this thoracoscopic approach utilizing the same catheter delivery system that we would propose to use transvenously allows for greater experience with the catheter and a respective cell and/or gene delivery. If endoscopic delivery is not possible in all animals, a mini (<6 cm) right thoracotomy will be performed and delivery still accomplished using the catheter.

Identification and quantification of autologous skeletal myoblasts and Cd133⁺ cells using immunohistochemical staining for My-32 and GFP labeled cells, respectively. As our preliminary data confirms, identification of skeletal muscle within the heart is reliably accomplished using the MY-32. We will attempt using both GFP labeled cells and co-staining with tissue specific antigens (connexin 43, CD45, GR-1, CD31) to verify the presence of injected Cd133⁺ cells within the scarred myocardium and their transdifferentiation.

Identification and quantification of neovascular formation in chronic scar will be accomplished using standard described techniques (Schuster et al. “Myocardial neovascularization by bone marrow angioblast results in cardiomyocyte regeneration” Am. J. Physiol. Heart Cir. 287:H525-H532, 2004; incorporated herein by reference). We will also utilize immunohistochemical staining for von Willebrand's factor (CD31) as another method of objectively identifying neovascularization (Pagani et al. “Autologous skeletal myoblasts transplanted to ischemia-damaged myocardium in humans” J. Am. Coll. Cardiol. 41:879-888, 2003; incorporated herein by reference).

### Example 3

### Treatment with Skeletal Myoblasts and VEGF in Sheep Model of Heart Failure

Anesthesia Protocol

Sheep are anesthetized for the procedures and surgeries described below. After sedation with an intramuscular (IM) injection of telazol, a catheter is placed into the dorsal ear vein or jugular vein for administration of thiopental (2-4 mg/lb IV) or etomidate (0.75-1.5 mg/lb IV) for anesthetic induction. An intravenous (IV) antibiotic injection of cefazolin (1.0 gm/5 mL), cefoxitin (1.0 gm/10 mL), and/or vancomycin (1.0 gm/10 mL) is administered. Orotracheal intubation is performed and anesthesia is maintained with 1-3% isoflurane and 100% oxygen. Positive pressure ventilation (10-15 ml/kg) and maintenance IV fluids (0.9% NaCl or lactated Ringer's solution {fourth root} 10 cc/kg/hr) are maintained. A fentanyl bolus and subsequent drip is administered concurrent with isoflurane administration to provide additional analgesia during the surgeries.

The following drugs are given IV as needed: potassium chloride (20-40 mEq=10-20 mL), calcium chloride (0.5-1.0 g=5-10 mL), magnesium chloride (0.5-2 g=1-4 mL), sodium bicarbonate (5-50 mEq=5-50 mL), phenylephrine (0.1-1 mg=diluted in saline =0.1-1.0 mL), dobutamine (as a drip to effect 0.125 mg/mL=5-50 mL/hr), epinephrine (0.1-1.0 mg=0.1-1.0 mL), lidocaine (20-60 mg=1-3 mL).

Sheep are positioned in lateral recumbency appropriate for the procedure or surgery to be performed. ECG leads are affixed for cardiac monitoring. Surgical sites are clipped free of hair prior to sterile preparation of the sites with betadine. All procedures are carried out under sterile (prepped and draped) conditions. During the minimally invasive surgical procedures arterial blood samples (0.5-3.0 mL) may be collected to evaluate blood gas and electrolyte status.

Minor Procedures

Sheep are anesthetized as above and undergo one or more of the procedures listed below. When possible, multiple procedures are performed at the same time to minimize the number of anesthetic events (maximum 5) per animal. All procedures are carried out under sterile conditions.

1) Embolization Procedure: This procedure induces heart failure and creates the model for the study. 2-5 embolizations at 5-14 day intervals are needed to achieve and maintain a cardiac ejection fraction (EF) consistently below 35%, a clinical sign of heart failure. Bupivacaine (0.5%, 2-5 mL) and lidocaine (2%, 2-5 mL) are injected subcutaneously (SC) at the incision site for long term local analgesia. A small incision (2-3″) is made over the external jugular vein. Catheter introducers (6-8 fr) are placed in the jugular vein and the carotid artery to facilitate placement of cardiac angiography catheters. Lidocaine (40 mg=2 mL IV) and/or MgSO₄ (2 mg=4.0 mL IV) is given to prevent or limit arrhythmias. Heparin (3-5,000 units=3-5 mL IV) is given to prevent clot formation. IV beta blockade (metoprolol 1-3 mg=1-3 mL, propranolol 1-3 mg=1-3 mL, isoproteronol 0.2-1 mg=1-5 mL, or ICI 8-10 mg=3-5 mL) may be used in animals as necessary. Accepted coronary angiographic techniques are employed. Selective left circumflex coronary artery embolizations are performed via the administration of (0.5-2.0 mL) 90 micron polystyrene beads. All catheters and introducers are removed when embolization and data collection is complete at the end of each procedure. The incision is closed in layers and a sterile dressing is applied.

2) Echocardiogram: A two-dimensional echocardiogram is performed with the sheep in right sternolateral recumbency. Images are stored on videotape for later analysis and assessment of ejection fraction (EF) and segmental left ventricular (LV) wall thickness and function.

3) Muscle Biopsy: A small incision (2 cm) is made over the left triceps muscle to expose it. An incisional biopsy (0.5 cm3) is taken and the cells are cultured and prepared for subsequent injection in animals assigned to ASM groups. The wound is closed in two layers (including the skin) using absorbable suture. A sterile dressing is applied.

4) Left Ventricular Angiogram: A left ventriculogram is performed to assess left ventricle (LV) function. Contrast dye solution (20-60 mL) is injected through a 5-7 fr pigtail catheter inserted through an introducer sheath in left carotid artery. Images are recorded (VCR tape) for later assessment of cardiac EF and segmental cardiac function.

5) Endomyocardial Biopsy: Specimens are obtained via endovascular biopsy forceps passed into the heart thru an 8 fr introducer sheath in the left jugular. Five specimens (5.0 mm³) are collected and frozen for later analysis.

6) Left Heart Catheterization and Hemodynamics: A 5-7 Fr pigtail pressure catheter is inserted into the LV through an introducer sheath in the L carotid artery for measurement of LV pressure. Data is acquired and analyzed using offline analysis software.

7) Right Heart Catheterization/Cardiac Output: Central venous pressure and pulmonary artery (PA) pressures are obtained from a Swan-Ganz catheter inserted through the jugular introducer. The catheter is connected to a fluid filled pressure transducer and CO is measured by thermodilution using 5 cc injections of cold 5% dextrose solution.

8) Collection of Blood Specimens: 25 cc of blood is collected for basic laboratory tests and analysis of serum cytokines and other systemic markers of heart failure (ET-1, PNE, etc.).

Minimally Invasive VEGF Administration

The angiogenic drug, VEGF, is administered directly to the heart via a minimally invasive mini-thoracotomy (incision <6 cm) or by thoracoscopic access. These are the same means by which the drug is expected to be administered to a human patient. This research will help to determine which approach is most appropriate. Minimally invasive techniques allow for relatively short anesthetic periods (<1 hour) and quick post operative recovery. Surgery is performed under general anesthesia and under sterile conditions. Groups 1-5 have a right mini-thoracotomy (small incision at 3-4th intercostal space), and Groups 6-8 have right thoracoscopic access (3-4 one inch intercostal incisions on the right chest wall) using an endoscope and endoscopic instruments. Bupivacaine (0.5%, 5 mL) and lidocaine (2%, 5 mL) are injected at the incision site to provide local long term analgesia. A single injection of cisatracurium (0.25 mg/kg=1.5-2.5 mL), a short acting neuromuscular blocking agent, is administered IV prior to the incisions, but only after a surgical depth of anesthesia is established.

Regional ischemia in heart failure animals (Groups 2-8) is confirmed by discoloration of myocardium and potential changes in cardiac rhythm. Animals in Group 1 receive treatment in the ischemic target area of the heart. Each sheep receives either AdVGEF (1×1010 pfu; the angiogenic growth factor carried by an adenovirus) or AdNull (an adenovirus with an empty expression cassette) injected in the area of myocardial infarction. Each animal receives 1-5 injections (0.2-3.0 mL/injection) using a 25 ga needle. Lidocaine (40-60 mg=2-3 mL) is administered IV as needed to treat ventricular arrhythmias that arise as a result of cardiac manipulation.

A chest tube will be placed, passing subcutaneously and exiting the right lateral chest. The minithoracotomy (Groups 1-6) will be closed in layers using permanent and absorbable suture as appropriate. The thoracoscopic incisions (Groups 7-8) will also be closed in standard fashion. Air will be evacuated from the chest cavity, the tube will be pulled and the incision closed. Animals will be allowed to recover under supervision. Ketorolac (0.2 mg/lb IM) and buprenorphine (0.05 mg/kg SC, 0.05 mL, q 8-12 h) will be administered to provide postoperative analgesia. A fenatanyl patch (50 mcg/hr) may be applied to provide additional analgesia following the immediate post operative period, although this may not be necessary with such minimally invasive procedures. All sheep will receive another dose of antibiotics: cefazolin (1.0 g/5 mL), cefoxitin (1.0 g/10 mL), and/or vancomycin (1.0 g/10 mL) given IV. Additional post operative care will be provided as outlined in the protocol below.

A utologous Skeletal Myoblast Administration and Instrumentation

Approximately three weeks after VEGF administration sheep are anesthetized, and the left chest is prepped with betadine and draped in a sterile fashion. A single injection of a short-acting neuromuscular blocking agent (cisatracurium, 0.25 mg/kg=1.5-2.5 mL IV) is given after a surgical depth of anesthesia has been established. Long term local analgesics (bupivacaine, 0.5%, 5 mL; and lidocaine 2%, 5 mL) are injected at the incision site. A left lateral thoracotomy is performed thru the 5th intercostal space, with or without 5th rib resection. A hydraulic occluder (14-20 mm) is positioned around the inferior vena cava. A set of six piezoelectric crystals is secured on the endocardial and epicardial surfaces of the heart. An aortic flow probe (14-20 mm) may also be placed to monitor blood flow. A calibrated dual pressure telemetry device (3.5 cm×1 cm) is implanted subcutaneously on the chest, allowing “hands-free” monitoring and data collection of cardiac parameters (e.g., ECG, pressure) in the postoperative period. Sealed pressure catheters (4 fr) from the telemetry device are placed and secured in the descending thoracic aorta and the left ventricle. Another fluid filled catheter is placed in the right ventricle to facilitate blood sample collection and therapeutic drug administration in the postoperative period, thus avoiding the use of needles for blood specimen collection. A series of left ventricular pacing leads (2-6) is placed to facilitate the measurement of myocardial impedance in the post operative period. Lidocaine (40-60 mg=2-3 mL) is used if needed to treat ventricular arrhythmias that may arise as a result of cardiac manipulation. The autologous skeletal myoblast (ASM) or control vehicle injections are then given; 1-5 injections (0.2-3.0 mL/injection) per animal administered via a 25 ga needle into the LV at various locations within the area of ischemic injury.

All catheters and instruments exit the chest and skin dorsally between the animal's scapula. Baseline hemodynamic measurements are taken prior tp closure of the chest to ensure all instrumentation is functional. A chest tube is placed, passed subcutaneously and exiting the left lateral chest. The chest is closed in layers using permanent and absorbable suture as appropriate. Bulb suction is applied to the drain. Ketorolac (0.2 mg/lb IM) and buprenorphine (0.005 mg/kg IM=1-2 mL, q 8-12 h) are administered to provide postoperative analgesia. All sheep receive antibiotics (ABs): cefazolin (1.0 g/5 mL), cefoxitin (1.0 g/10 mL), and/or vancomycin (1.0 g/10 mL) IV.

The chest is bandaged and covered with a “jacket” to protect the incisions and instrumentation from inadvertent injury. Animals recover from anesthesia under supervision. After the ET tube is removed and the animal can maintain spontaneous ventilation, the dorsal ear vein catheter is removed. The sheep is returned to the vivarium animal housing facility and routine husbandry. Sheep recover more quickly and with less stress when they are within sight of other sheep. Research personnel continue to monitor the sheep every 1-2 hours until the animal is eating hay and drinking water without asistance.

Post Operative Care

Sheep are given analgesics (buprenorphine=0.005 mg/kg IM=1-2 mL, q 8-12 h, and/or a fenatanyl patch =50 mcg/hr for 72 hr) for long term analgesia in the post operative period. Antibiotics (same as above) are administered IV every 8-12 hours (as dictated by type) and may be continued post operatively for up to 2 weeks. The chest tube is evacuated every 4-8 hours and remains in place for up to 48 hours. Withdrawn fluid is evaluated for consistency and volume. Surgical sites, catheters, and bandages are monitored daily and changed as needed, or every 2-5 days throughout the course of the study. Animal care staff record the weights and temperatures of the sheep every 5-10 days and notify research personnel of any significant changes.

Data Collection

Physiologic data (heart rate, blood pressure, blood flow, etc.) is collected every 1-14 days (typically twice a week) following ASM administration. A transport stanchion/cart is used to provide a safe environment for the sheep both during transport to the data collection room (next to the housing room within the vivarium) and during data collection. Sedation and physical restraint of the sheep are not necessary.

Monitoring instrumentation is connected to the data acquisition system for collection of data. An inotropic agent, such as dobutamine (1-10 mcg/kg/min =1-50 mL−study time period varies with each individual, lasting from 5-25 minutes or less), may be administered intravenously. This is a clinically acceptable method of assessing cardiac function. Beta receptor blockade may also be initiated through the IV administration of agents such as metoprolol (1-3 mg=1-3 mL), propranolol (1-3 mg=1-3 mL), or ICI (8-10 mg=3-5 mL) in order to evaluate the compensatory ability of the failing heart in the presence of a beta adrenergic compound such as isoproteronol (0.2-1 mg=1-5 mL). Changes in receptor availability and sensitivity may be related to the progression of heart failure in the animal.

Blood samples (25 mL) may be drawn for analysis of basic laboratory tests, serum cytokines and other systemic markers of heart failure (ET-1, PNE, etc.). Lab tests do not exceed 3 per week, unless the animal's welfare requires more frequent testing. Any necessary medications are administered at this time, and the ventricular catheters are flushed with heparin (1000 U/ml, 3-5 mL). The surgical incisions are inspected (as stated in post operative care) and the bandages are changed. The jacket is put over the bandages and the sheep is returned to the housing room.

Terminal Procedure

A final data collection event occurs 6 weeks following the ASM administration. The sheep is anesthetized as before and immediately euthanized with IV saturated potassium chloride. Explanted tissues will be used for further in vitro study with some being either frozen or fixed in <10% buffered formalin solution and subsequently prepared for histological analysis.

Quantitation of Cell Survival

The heart at the injection sites is cut into blocks approximately 2.5 mm×2.5 mm×0.3 mm in dimension and processed in paraffin. The tissue is then cut at a thickness of 5 μm and placed on slides for histological analysis. In some cases, the whole block is sectioned, in other cases only a portion of the tissue is sectioned. Tissue sections are then stained with H&E, or Trichrome and immunostained for skeletal-specific myosin heavy chain (MY32), myogenin, or myoD.

To approximate the survival of myoblasts in the heart, the area of the graft(s) in a representative tissue section and the density of nuclei per graft area are determined. The following equation is then used to determine the total number of surviving myoblast nuclei in the tissue block.

$$\begin{matrix}Sum of Graft \\ Area in Section\end{matrix} \times \begin{matrix}Density of Nuclei \\ Per Graft Area\end{matrix} \times \begin{matrix}\# Sections \\ Per Block\end{matrix} \times \begin{matrix}Abercrombie \\ Correction ( 1 )\end{matrix}$$

For Example:

$$2.675 \times {10}^{6} {µm}^{2} \times 6.3 \times {10}^{- 4} nuclei / {µm}^{2} \times 600 \times 0.45 = 4.6 \times {10}^{5} myoblast nuclei in the tissue block$$

The Abercrombie corrrection adjusts for the possibility of counting the same nucleus in adjacent sections.

The calculated cell number from all blocks with grafts is then divided by the number of myoblasts injected to determine the percent survival.

Results

Nine sheep were transplanted with ≦400 million cells. The survival percentage is shown in Table 6 below for each animal. Survival percentages were higher on average in the sheep that received VEGF prior to cell transplant. These data support the hypothesis that angiogenic factors can improve the overall survival of transplanted cells.

TABLE 6
Myoblast Survival

| Myoblasts | Myoblasts + VEGF |
|---|---|
| 2.10% | 18.00% |
| 2.30% | 8.60% |
| 0.10% | 2.50% |
| 0.05% | 1.60% |
| 10.70% | |
| 3.00% | 7.60% Ave. |

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A method for treating heart disease, the method comprising steps of:
administering a pro-angiogenic agent to a patient suffering from heart disease; and
administering a composition of cells to the heart of the patient.

2. The method of claim 1, wherein the step of administering the pro-angiogenic agent is performed before the step of administering the composition of cells.

3. The method of claim 1, wherein the step of administering the pro-angiogenic agent is repeated at least twice.

4. The method of claim 1, wherein the step of administering the composition of cells is repeated at least twice.

5. The method of claim 1, wherein the pro-angiogenic agent is a protein or peptide.

6. The method of claim 1, wherein the pro-angiogenic agent is a small molecule.

7. The method of claim 1, wherein the pro-angiogenic agent is a polynucleotide.

8. The method of claim 1, wherein the pro-angiogenic agent is a cell.

9. The method of claim 1, wherein the pro-angiogenic agent is an endothelial cell, an endothelial stem cell, a bone marrow-derived stem cell, an embryonic stem cell, cord blood cells, a primordial germ cell, a neural stem cell, a pluripotent stem cell, a skeletal myoblast, or a mesenchymal stem cell.

10. The method of claim 1, wherein the pro-angiogenic agent is selected from the group consisting of vascular endothelial growth factor (VEGF), angiogenin, growth factors, hypoxia-inducible factor-1 (HIF-1), epidermal growth factor (EGF), bFGF, angiopoietin, acidic fibroblast growth factor (FGF-1), basic fibroblast growth factor (FGF-2), platelet-derived growth factor, angiogenic factor, transforming growth factor-alpha (TGF-α), transforming growth factor-beta (TGF-β), vascular permeability factor (VPF), tumor necrosis factor alpha (TNF-α), interleukin-3 (IL-3), interleukin-8 (IL-8), platelet-derived endothelial growth factor (PD-EGF), granulocyte colony stimulating factor (G-CSF), hepatocyte growth factor (HGF), scatter factor (SF), pleitrophin, proliferin, follistatin, placental growth factor (PIGF), midkine, platelet-derived growth factor-BB (PDGF), and fractalkine.

11. The method of claim 1, wherein the pro-angiogenic agent is vascular endothelial growth factor (VEGF).

12. The method of claim 1, wherein the heart disease is coronary heart disease, chronic heart failure, ischemic heart disease, congestive heart failure, cardiomyopathy, dilated cardiomyopathy, viral cardiomyopathy, or myocardial infarction.

13. The method of claim 1, wherein the composition of cells comprises a viscosity enhancing agent.

14. The method of claim 1, wherein the composition of cells comprises a polymer.

15. The method of claim 1, wherein the composition of cells comprises a matrix.

16. The method of claim 1, wherein the cells are skeletal myoblasts.

17. The method of claim 1, wherein the cells are stem cells.

18. The method of claim 1, wherein the cells are embryonic stem cells or bone marrow stem cells.

19. The method of claim 1, wherein the cells are mesenchymal stem cells.

20. The method of claim 1, wherein the cells are mesenchemymal stem cells that have been cultured with fetal cardiomyocytes.

21. The method of claim 1, wherein the cells are fetal cardiomyocytes.

22. The method of claim 1, wherein the cells are human cells.

23. The method of claim 1, wherein the cells have been cultured.

24. The method of claim 1, wherein the cells have been minimally cultured.

25. The method of claim 1, wherein the cells have not doubled in vitro.

26. The method of claim 1, wherein the cells are not been cultured.

27. The method of claim 1, wherein the cells are derived from the patient.

28. The method of claim 1, wherein the cells are derived from a human donor.

29. The method of claim 1, wherein the cells express an anti-apoptotic factor.

30. The method of claim 1, wherein the cells express Akt.

31. The method of claim 1, wherein the cells express a growth factor.

32. The method of claim 1, wherein the cells express a basic fibroblast growth factor (bFGF).

33. The method of claim 1, wherein the step of administering the agent is performed at least 1 week before the step of administering the composition of cells.

34. The method of claim 1, wherein the step of administering the agent is performed at least 2 weeks before the step of administering the cells.

35. The method of claim 1, wherein the step of administering the agent is performed at least 3 weeks before the step of administering the cells.

36. The method of claim 1, wherein the step of administering the agent is performed at least 4 weeks before the step of administering the cells.

37. The method of claim 1, whereby the method improves the exercise tolerance of the patient two weeks after administration of the composition of cells.

38. The method of claim 1, whereby the method increases cardiac output two weeks after administration of the composition of cells.

39. The method of claim 1, whereby the method decreases cardiac dilation.

40. The method of claim 1, whereby the method leads to an attentuation of left ventricular dilation as measured by left ventricular end-systolic volume index.

41. A method for the treatment of heart disease, the method comprising steps of:
administering a vector comprising a polynucleotide encoding a pro-angiogenesis factor to the heart of a patient suffering from heart disease; and
administering a composition of cells to the heart of the patient.

42. The method of claim 41, wherein the step of administering the vector is performed before the step of administering the cells.

43. The method of claim 41, wherein the heart disease is coronary artery disease, congestive heart failure, chronic heart failure, ischemic heart disease, a cardiomyopathy, dilated cardiomyopathy, or myocardial infarction.

44. The method of claim 41, wherein the cells are skeletal myoblasts.

45. The method of claim 41, wherein the cells are stem cells or embryonic stem cells.

46. The method of claim 41, wherein the cells are mesenchymal stem cells.

47. The method of claim 41, wherein the cells are mesenchymal stem cells cultured with fetal cardiomyocytes.

48. The method of claim 41, wherein the cells are fetal cardiomyocytes.

49. The method of claim 41, wherein the cells are bone marrow stem cells.

50. The method of claim 41, wherein the cells are derived from the patient.

51. The method of claim 41, wherein the cells are derived from a human donor.

52. The method of claim 41, wherein the cells express anti-apoptotic factors.

53. The method of claim 41, wherein the cells express Akt.

54. The method of claim 41, wherein the cells express a growth factor.

55. The method of claim 41, wherein the cells express basic fibroblast growth factor (bFGF).

56. The method of claim 41, wherein the cells are mesenchymal stem cells.

57. The method of claim 41, wherein the vector comprises DNA.

58. The method of claim 41, wherein the vector comprises RNA.

59. The method of claim 41, wherein the vector encodes a pro-angiogenic factor selected from the group consisting of vascular endothelial growth factor (VEGF), angiogenin, growth factors, hypoxia-inducible factor-1 (HIF-1), epidermal growth factor (EGF), bFGF, angiopoietin, acidic fibroblast growth factor (FGF-1), basic fibroblast growth factor (FGF-2), platelet-derived growth factor, angiogenic factor, transforming growth factor-alpha (TGF-α), transforming growth factor-beta (TGF-β), vascular permeability factor (VPF), tumor necrosis factor alpha (TNF-60 ), interleukin-3 (IL-3), interleukin-8 (IL-8), platelet-derived endothelial growth factor (PD-EGF), granulocyte colony stimulating factor (G-CSF), hepatocyte growth factor (HGF), scatter factor (SF), pleitrophin, proliferin, follistatin, placental growth factor (PIGF), midkine, platelet-derived growth factor-BB (PDGF), and fractalkine.

60. The method of claim 41, wherein the vector is a plasmid, virus, adenovirus, or adeno-associated virus.

61. The method of claim 41, wherein the vector is an adenovirus or adeno-associated virus encoding VEGF.

62. The method of claim 41, wherein the vector is an adenovirus or adeno-associated virus encoding VEGF₁₂₁.

63. The method of claim 41, wherein the vector provides constitutive expression of an angiogenic factor.

64. The method of claim 41, wherein the vector provides hypoxia-induced expression of an angiogenic factor.

65. The method of claim 63 or 64, wherein the angiogenic factor is VEGF.

66. The method of claim 41, wherein the step of administering the vector is performed at least 1 weeks before the step of administering the cells.

67. The method of claim 41, wherein the step of administering the vector is performed at least 2 weeks before the step of administering the cells.

68. The method of claim 41, wherein the step of administering the vector is performed at least 3 weeks before the step of administering the cells.

69. The method of claim 41, wherein the step of administering the vector is performed at least 4 weeks before the step of administering the cells.

70. The method of claim 41, wherein the step of administering the vector is performed at least 6 weeks before the step of administering the cells.

71. The method of claim 41, wherein the step of administering the vector is performed at least 8 weeks before the step of administering the cells.

72. The method of claim 41, whereby there is at least a two-fold increase in capillary density 3 weeks after the step of administering the vector.

73. The method of claim 41, wherein the step of administering the cells comprising administering the cells to the heart via a catheter.

74. A method for treating heart disease, the method comprising step of:
administering the cells to the heart of a patient suffering from heart disease,
wherein the cells are selected from the group consisting of skeletal myoblasts, fetal cardiomyocytes, embryonic stem cells, mesenchymal stem cells, or bone marrow stem cells; and
wherein the cells are engineered to express an pro-angiogenic factor.

75. The method of claim 74, wherein the cells are skeletal myoblasts.

76. The method of claim 74, wherein the cells are mesenchymal stem cells.

77. The method of claim 74, wherein the pro-angiogenic factor is selected from the group consisting of vascular endothelial growth factor (VEGF), angiogenin, growth factors, hypoxia-inducible factor-1 (HIF-1), epidermal growth factor (EGF), bFGF, angiopoietin, acidic fibroblast growth factor (FGF-1), basic fibroblast growth factor (FGF-2), platelet-derived growth factor, angiogenic factor, transforming growth factor-alpha (TGF-α), transforming growth factor-beta (TGF-β), vascular permeability factor (VPF), tumor necrosis factor alpha (TNF-α), interleukin-3 (IL-3), interleukin-8 (IL-8), platelet-derived endothelial growth factor (PD-EGF), granulocyte colony stimulating factor (G-CSF), hepatocyte growth factor (HGF), scatter factor (SF), pleitrophin, proliferin, follistatin, placental growth factor (PIGF), midkine, platelet-derived growth factor-BB (PDGF), and fractalkine.

78. A kit comprising (1) a pro-angiogenic factor; and (2) skeletal myoblasts or mesenchymal stem cells.

79. The kit of claim 78 further comprising a needle, a syringe, a catheter, and a pharmaceutically acceptable excipient for suspending the myoblasts in.

80. The kit of claim 78, wherein the needle is side port needle.

81. The kit of claim 78, wherein the skeletal myoblasts or mesenchymal stem cells are genetically engineered to express a pro-angiogenic factor.

82. The kit of claim 78, wherein the pro-angiogenic factor is selected from the group consisting of angiogenin, growth factors, hypoxia-inducible factor-1 (HIF-1), epidermal growth factor (EGF), bFGF, angiopoietin, acidic fibroblast growth factor (FGF-1), basic fibroblast growth factor (FGF-2), platelet-derived growth factor, angiogenic factor, transforming growth factor-alpha (TGF-α), transforming growth factor-beta (TGF-β), vascular permeability factor (VPF), tumor necrosis factor alpha (TNF-α), interleukin-3 (IL-3), interleukin-8 (IL-8), platelet-derived endothelial growth factor (PD-EGF), granulocyte colony stimulating factor (G-CSF), hepatocyte growth factor (HGF), scatter factor (SF), pleitrophin, proliferin, follistatin, placental growth factor (PIGF), midkine, platelet-derived growth factor-BB (PDGF), vascular endothelial growth factor (VEGF), and fractalkine.

83. The kit of claim 78, wherein the pro-angiogenic factor is vascular endothelial growth factor.
